(19) United States
(12) Patent Application Publication
(10) Pub. No.: US 2026/0258441 A1
(43) Pub. Date: Sep. 3, 2026

(54) ENGINEERED HYPOXIA BIOSENSORS AND METHODS OF USING THE SAME

(71) Applicant: Northwestern University, Evanston, IL (US)

(72) Inventors: Yannick Rene SCHREIBER, Chicago, IL (US); Joshua N. LEONARD, Evanston, IL (US); Patrick Sean DONAHUE, Evanston, IL (US)

(73) Assignee: Northwestern University, Evanston, IL (US)

(21) Appl. No.: 18/993,319

(22) PCT Filed: Jul. 11, 2023
(86) PCT No.: PCT/US2023/027405

§ 371 Date: Jan. 10, 2025

Related U.S. Application Data
(60) Provisional application No. 63/388,530, filed on Jul. 12, 2022.

(51) Int. Cl. C12N 15/85; C12N 15/90

(52) U.S. Cl. CPC C12N 15/85; C12N 15/907; C12N 2800/90; C12N 2830/002

(57) ABSTRACT

The present disclosure relates generally to hypoxia biosensors. In particular, the present disclosure relates to hypoxia biosensors that employ engineered genetic circuits for enhanced biosensor sensitivity and response. Some engineered genetic circuits employ positive feedback and amplification of expression in order to promote high-efficiency reporting of hypoxia.

### CROSS-REFERENCE TO RELATED APPLICATIONS

This application is the U.S. National Stage of PCT/US2023/027405, filed Jul. 11, 2023, which claims the benefit under 35 U.S.C. § 119(e) of U.S. Provisional Patent Application No. 63/388,530, filed Jul. 12, 2022, the entire contents of which is incorporated herein by reference in its entirety.

### GOVERNMENT SUPPORT

This invention was made with government support under grant numbers 1R01EB026510 and 1F30CA203325-02 awarded by the National Institutes of Health (NIH), and grant number MCB-1745753 awarded by the National Science Foundation (NSF). The government has certain rights in the invention.

The instant application contains a Sequence Listing which has been submitted in XML format and is hereby incorporated by reference in its entirety. Said XML copy, created on Sep. 8, 2023, is named 121384-0208.xml and is 145,974 bytes.

### FIELD OF INVENTION

The present disclosure relates generally to hypoxia biosensors employing engineered genetic circuits. In particular, the present disclosure relates to hypoxia biosensors and methods of using the same, as well as novel engineered genetic circuits that incorporate signal amplification and positive feedback in response to a stimulus (e.g., hypoxia) for enhanced biosensor sensitivity.

### BACKGROUND

The following discussion is merely provided to aid the reader in understanding the disclosure and is not admitted to describe or constitute prior art thereto.

Hypoxia is a pathologic condition in which a tissue is not exposed to sufficient levels of oxygen. Hypoxia is a feature in many tumors. As growing tumors require a steady supply of nutrients for their continued proliferation, they produce vascular endothelial growth factor (VEGF) to induce the growth of blood vessels. However, tumor growth often surpasses the rate at which new blood vessels can grow, and the vasculature in tumors is markedly abnormal, both of which may lead to poor perfusion and resultant hypoxia. Tumor vasculature is elongated and tortuous, limiting the delivery of fresh blood, in contrast to well-organized physiologic vascular networks. Further, the tight barrier formed by endothelial and smooth muscle cells is frequently insufficient, resulting in unusually permeable vasculature, which affects the ability to maintain adequate perfusion. Together, these effects lead to regions of hypoxia throughout the tumor. While healthy tissues have multiple mechanisms to counteract hypoxia, including altering cellular metabolism and rapidly increasing blood flow through arterial dilatation, tumors lack the ability to respond likewise. This makes hypoxia a good marker for tumors in otherwise healthy humans, particularly those lacking ischemic disease.

A cell's response to hypoxia may include the stabilization of two hypoxia inducible factors (HIFs), HIF1α and HIF2α, as extracellular oxygen decreases. These transcription factors (TFs) can each heterodimerize with a constitutively stable HIF1β, bind to hypoxia response elements (HREs), and activate sets of genes that promote adaptation to and resolution of hypoxia. This pathway is also responsible for many adaptations that allow tumors to grow rapidly, and it is dysregulated in many tumors. This signaling process forms the basis for DNA-based hypoxia biosensors (HBSs). By placing these HREs upstream of a minimal promoter, a downstream gene of interest can be conditionally expressed only when a cell is experiencing hypoxic conditions. Additional elements can be placed upstream of, placed downstream of, or intermixed with the HREs to increase the response to hypoxia, as well as to small molecule mimetics of hypoxia. These sensors have enabled in vivo imaging of the response to hypoxia in mice. It has also been shown that the choice of minimal promoter can influence magnitude of this response, as well as the amount of gene expression under normoxic conditions. However, biosensors that rely solely on this endogenous response may not be robust to dysregulation of the hypoxia response, which occurs in many tumors as both a survival mechanism and a consequence of the tumor microenvironment (TME).

Accordingly, there is a need in the art for robust and sensitive hypoxia biosensors. The present disclosure fulfills that need by providing such hypoxia biosensors with a variety of hypoxia-sensing mechanisms.

### SUMMARY

The present disclosure provides DNA-based hypoxia biosensors with improved function. The disclosed hypoxia biosensors may be used in a variety of applications, including but not limited to, targeting cell-based therapies to tumor microenvironments, in which hypoxia is common. Further details and embodiments are described herein.

In one aspect, the present disclosure provides a DNA-based hypoxia biosensor, comprising: (a) a hypoxia-inducible promoter; (b) a nucleic acid sequence encoding a functional element; and (c) at least one nucleic acid sequence encoding a feedback element. In some embodiments, the hypoxia-inducible promoter comprises (i) an Egr-1-binding site (EBS) from a Egr-1 gene, a metal-response element (MRE) from a metallothionein gene, a hypoxia-response element (HRE), or a combination thereof, and (ii) a minimal promoter. In some embodiments, the hypoxia-inducible promoter comprises (i) an Egr-1-binding site (EBS) from a Egr-1 gene, a metal-response element (MRE) from a metallothionein gene, and at least one a hypoxia-response element (HRE), and (ii) a minimal promoter. In some embodiments, the at least one HRE is three HREs (EBS-MRE-3×HRE). In some embodiments, the minimal promoter is selected from simian virus 40 (SV40) promoter, cytomegalovirus (CMV) promoter, and a synthetic promoter. In some embodiments, the synthetic promoter is YB_TATA. In some embodiments, the functional element is a gene sequence or fragment thereof, a nucleic acid sequence encoding a regulatory RNA molecule, or a reporter element. In some embodiments, the functional element is a reporter element. In some embodiments, the reporter element is selected from a fluorophore, a luciferase, a peroxidase, and a combination thereof. In some embodiments, the combination comprises a fusion protein. In some embodiments, the fusion protein comprises a luciferase polypeptide fused to a fluorophore. In some embodiments, the feedback element is a positive feedback element. In some embodiments, the positive feedback element is selected from HIF1α and HIF2α. In some embodiments, the positive feedback element is either in-whole or in-part a gene that occurs in nature. In some embodiments, the positive feedback element is either in-whole or in-part derived from a gene that occurs in nature. In some embodiments, the positive feedback element is either in-whole or in-part a gene that is of synthetic origin. In some embodiments, the hypoxia biosensor comprises a nucleic acid sequence encoding a first positive feedback element, and a nucleic acid sequence encoding a second positive feedback element. In some embodiments, the first positive feedback element is selected from HIF1α and HIF2α, and the second positive feedback element is, optionally, HIF1β. In some embodiments, the functional element comprises at least one oxygen-degradation domain (ODD). In some embodiments, the functional element is a chimeric antigen receptor (CAR). In some embodiments, the nucleic acid sequence encoding a functional element and the at least one nucleic acid sequence encoding a feedback element are separated by a nucleic acid sequence encoding a cleavage peptide. In some embodiments, the cleavage peptide is a self-cleaving peptide, optionally a T2A peptide. In some embodiments, the nucleic acid sequence encoding a functional element is 5′ of the at least one nucleic acid sequence encoding a feedback element. In some embodiments, the nucleic acid sequence encoding a functional element is 3′ of the at least one nucleic acid sequence encoding a feedback element. In some embodiments, the hypoxia biosensor comprises at least two hypoxia-inducible promoters. In some embodiments, the nucleic acid sequence encoding a functional element and the at least one nucleic acid sequence encoding a feedback element are operably linked to separate hypoxia-inducible promoters. In some embodiments, the feedback element is HIF1α. In some embodiments, the feedback element is HIF2α.

In another aspect, the present disclosure provides a DNA-based hypoxia biosensor, comprising: (a) a hypoxia-inducible promoter; (b) a nucleic acid sequence encoding a functional element; (c) at least one nucleic acid sequence encoding one or more engineered proteins selected from the group consisting of: (i) an engineered protein that activates gene expression, wherein the engineered protein comprises a DNA binding domain and a transcription activator domain; (ii) an engineered protein that inhibits gene expression; (iii) an engineered protein that inhibits gene expression, the engineered protein optionally comprising one or more of a DNA binding domain, a bulky domain, a chromatin remodeling domain, and a transcription inhibitor domain; and (iv) a combination of two engineered proteins comprising a first engineered protein comprising a DNA binding domain fused to a dimerization domain, and a second engineered protein comprising a transcription regulator domain fused to a dimerization domain, wherein the dimerization domains of the two engineered proteins dimerize in the presence of a stimulus to which the dimerization domains of the two engineered proteins bind; and (d) one or more DNA binding sites for the DNA binding domain of the engineered protein(s) of (c). In some embodiments, the functional element is a reporter element. In some embodiments, the reporter is selected from a fluorophore, a luciferase, a peroxidase, and a combination thereof. In some embodiments, the combination comprises a fusion protein. In some embodiments, the fusion protein comprises a luciferase polypeptide fused to a fluorophore. In some embodiments, the engineered protein comprises a positive feedback element. In some embodiments, the hypoxia-inducible promoter comprises a minimal promoter selected from the group consisting of simian virus 40 (SV40) promoter, cytomegalovirus (CMV) promoter, and a synthetic promoter. In some embodiments, the synthetic promoter is YB_TATA. In some embodiments, the engineered protein is a COMET transcription factor (COMET TF). In some embodiments, the engineered protein comprises at least one oxygen-degradation domain (ODD). In some embodiments, at least one DNA binding site for the DNA binding domain of the engineered protein(s) is 5′ of the nucleic acid sequence encoding the functional element. In some embodiments, at least one DNA binding site for the DNA binding domain of the engineered protein(s) is 5′ of the hypoxia-inducible promoter. In some embodiments, at least one DNA binding site for the DNA binding domain of the engineered protein(s) is 5′ of the nucleic acid sequence encoding the functional element, and at least one DNA binding site for the DNA binding domain of the engineered protein(s) is 5′ of the hypoxia-inducible promoter. In some embodiments, at least one DNA binding site for the DNA binding domain of the engineered protein(s) is 5′ of the nucleic acid sequence encoding the functional element, and at least one DNA binding site for the DNA binding domain of the engineered protein(s) is 5′ of at least one nucleic acid sequence encoding the one or more engineered proteins. In some embodiments, at least one DNA binding site for the DNA binding domain of the engineered protein(s) is 5′ of at least one nucleic acid sequence encoding the one or more engineered proteins. In some embodiments, at least one DNA binding site for the DNA binding domain of the engineered protein(s) is 5′ of the nucleic acid sequence encoding the functional element; at least one DNA binding site for the DNA binding domain of the engineered protein(s) is 5′ of the hypoxia-inducible promoter; and at least one DNA binding site for the DNA binding domain of the engineered protein(s) is 5′ of at least one nucleic acid sequence encoding the one or more engineered proteins. In some embodiments, the biosensor comprises at least two nucleic acid sequences encoding one or more engineered proteins, and, optionally, wherein there is at least one DNA binding site for the DNA binding domain of the engineered protein(s) that is 5′ to each of the at least two nucleic acid sequences encoding the one or more engineered proteins. In some embodiments, the engineered protein(s) comprises at least one split intein on the C-terminus or N-terminus of the DNA binding domain, a transcription activator domain, or a combination thereof.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1A (FIG. 1A) is a schematic illustrating the composition of the hypoxia biosensor DNA sequence.

FIG. 1B (FIG. 1B) is a schematic illustrating the hypoxia biosensor with a downstream DsRed-Express2 reporter protein. The hypoxia biosensor is activated by HIF1α and HIF2α, which are degraded in the presence of oxygen.

FIG. 1C (FIG. 1C) is a graph showing results of experiments described herein. B16F10 cells with hypoxia biosensors integrated into the genome were cultured under normoxic or hypoxic conditions and reporter expression analyzed by flow cytometry. The x-axis shows the different hypoxia biosensor constructs tested, including three different minimal promoters (SV40, CMV, YB_TATA), and whether the cell contained extra productive copies or non-productive copies of the hypoxia biosensor. Non-productive copies of the hypoxia biosensor are termed non-productive as they do not have a downstream reporter gene. The results show that hypoxia biosensor performance is not enhanced by increasing the number of biosensors in the cell.

FIG. 2 (FIG. 2) shows an annotated map (A) and DNA sequence (B) (SEQ ID NO: 14) of a hypoxia biosensor with the YB_TATA minimal promoter.

FIG. 3 (FIG. 3) shows annotated map of a plasmid containing a hypoxia biosensor with the YB_TATA minimal promoter producing a LumiScarlet (mScarlet-I and LumiLuc fusion protein) reporter protein.

FIG. 4A (FIG. 4A) is a schematic illustrating a circuit in which doxycycline induces the production of stable HIF1α, which drives reporter gene expression from the hypoxia biosensor.

FIG. 4B (FIG. 4B) is a graph showing results of experiments described herein. HEK293FT cells with the circuit depicted in FIG. 4A were cultured for 5 days under one of the four conditions shown and analyzed daily with flow cytometry.

FIG. 4C (FIG. 4C) is a panel of plots showing results of experiments described herein. HEK293FT cells with the circuit depicted in FIG. 4A were cultured for 5 days under one of the four conditions shown and analyzed daily with flow cytometry. The results show that mutant stable HIF1α leads to higher levels of gene expression from the hypoxia biosensor than does either culture under hypoxic conditions or treatment with the hypoxia mimetic cobalt.

FIG. 5A (FIG. 5A) is a schematic illustrating a hypoxia biosensor that is enhanced by positive feedback with HIF1α.

FIG. 5B (FIG. 5B) is a schematic illustrating a hypoxia biosensor that is enhanced by positive feedback with HIF1α and HIF1β.

FIG. 5C (FIG. 5C) is a graph showing the results of experiments described here. B16F10 cells with hypoxia biosensors with or without several feedback strategies integrated into their genomes were cultured under normoxic or hypoxic conditions and reporter expression was then analyzed by flow cytometry. Fold induction is shown over the hypoxic bar. Each cell line tested had a hypoxia biosensor with the YB_TATA minimal promoter. All feedback designs tested increased the level of hypoxia-induced gene expression from the hypoxia biosensor.

FIG. 5D (FIG. 5D) is a panel of schematics and graphs showing results of experiments described herein. B16F10 cells with hypoxia biosensors with or without several feedback strategies integrated into their genomes were cultured under normoxic or hypoxic conditions and reporter expression analyzed by flow cytometry daily for up to 5 days. The results show that hypoxia biosensor performance can be enhanced with positive feedback with HIF1α and HIF2α.

FIG. 6 (FIG. 6) shows an annotated map of a DNA sequence containing a hypoxia biosensor without enhancement.

FIG. 7 (FIG. 7) shows an annotated map of a DNA sequence containing a hypoxia biosensor enhanced by positive feedback with HIF1α.

FIG. 8 (FIG. 8) shows an annotated map of a DNA sequence containing a hypoxia biosensor enhanced by positive feedback with HIF2α.

FIG. 9 (FIG. 9) shows an annotated map of a DNA sequence containing a hypoxia biosensor enhanced by positive feedback with HIF1α and HIF1β.

FIG. 10 (FIG. 10) shows an annotated map of DNA sequence containing a hypoxia biosensor enhanced by positive feedback with HIF2α and HIF1β.

FIG. 11 (FIG. 11) shows an annotated map (A) and DNA and protein sequences (B) (SEQ ID NO: 15) of a COMET transcription factor.

FIG. 12 (FIG. 12) shows an annotated map (A) and DNA and protein sequences (B) (SEQ ID NO: 16) of a COMET transcription factor with oxygen degradation tags on the N-terminus, in the linker, and on the C-terminus.

FIG. 13 (FIG. 13) shows an annotated map (A) and DNA sequence (B) (SEQ ID NO: 17) of a COMET promoter with binding sites for a COMET transcription factor.

FIG. 14 (FIG. 14) shows an annotated map of a COMET reporter plasmid with a COMET promoter producing the LumiScarlet reporter protein.

FIG. 15A (FIG. 15A) is a schematic illustrating a hypoxia biosensor with its output amplified by a COMET transcription factor that is destabilized in the presence of oxygen.

FIG. 15B (FIG. 15B) is a graph showing results of experiments evaluating COMET TFs with oxygen degradation motifs in various locations and copy numbers in HEK293FT cells by transient transfection. Cells were transfected with the COMET transcription factors, tagged with oxygen degradation domains as depicted on the x-axis, as well as a reporter plasmid from which the COMET transcription factor drives reporter gene expression. All tag locations and copy numbers led to higher levels of COMET-induced gene expression when cells were cultured under hypoxic conditions than when cultured under normoxic conditions.

FIG. 15C (FIG. 15C) is a graph showing results of experiments evaluating the top performing COMET TFs in B16F10 cells by transient transfection, performed as described in FIG. 15B. All tag locations and copy numbers led to higher levels of COMET-induced gene expression when cells were cultured under hypoxic conditions than when cultured under normoxic conditions.

FIG. 15D (FIG. 15D) is a panel of representative flow cytometry plots showing changes in constitutive (miRFP720) and COMET transcription factor-induced reporter (mNeonGreen) gene expression in two cell lines between normoxic and hypoxic culture. The results show that oxygen degradation tags confer oxygen sensitivity to COMET TFs.

FIG. 16A (FIG. 16A) is a schematic illustrating a hypoxia biosensor that is unenhanced.

FIG. 16B (FIG. 16B) is a schematic illustrating a hypoxia biosensor which is enhanced by signal amplification by a COMET transcription factor (ZFa).

FIG. 16C (FIG. 16C) is a schematic illustrating a hypoxia biosensor which is enhanced by signal amplification by a COMET transcription factor (ZFa), in which the COMET transcription factor also induces expression of more of itself from a separate transcription unit (termed “subsequent positive feedback”).

FIG. 16D (FIG. 16D) is a schematic illustrating a hypoxia biosensor which is enhanced by signal amplification by a COMET transcription factor (ZFa), in which the COMET transcription factor also induces expression of more of itself by binding to a locus upstream of the hypoxia biosensor (termed “positive feedback”).

FIG. 16E (FIG. 16E) is a schematic illustrating a hypoxia biosensor which is enhanced by signal amplification by a COMET transcription factor (ZFa), in which the COMET transcription factor also induces expression of more of itself by employing both the subsequent positive feedback and positive feedback strategies.

FIG. 16F (FIG. 16F) is a graph showing results of experiments described herein. B16F10 cells with hypoxia biosensors with or without several feedback strategies integrated into their genomes were cultured under normoxic or hypoxic conditions and reporter expression analyzed by flow cytometry. Several evaluated strategies enable hypoxia sensing regardless of the type of COMET transcription factor used (b), others only sensed hypoxia only when the COMET transcription factor was oxygen-sensitive (c, e), and others sensed hypoxia with the regular COMET transcription factor but were enhanced when it was oxygen-sensitive. The results show that hypoxia biosensor performance can be enhanced with COMET transcription factors.

FIG. 17 (FIG. 17) shows an annotated map (A) and DNA sequence (B) (SEQ ID NO: 18) of a hypoxia biosensor with upstream binding sites for a COMET transcription factor.

FIG. 18 (FIG. 18) shows an annotated map of a DNA sequence containing a hypoxia biosensor enhanced by amplification with COMET transcription factors.

FIG. 19 (FIG. 19) shows an annotated map of a DNA sequence containing a hypoxia biosensor enhanced by amplification with COMET transcription factors and subsequent positive feedback.

FIG. 20 (FIG. 20) shows an annotated map of a DNA sequence containing a hypoxia biosensor enhanced by amplification with COMET transcription factors and positive feedback.

FIG. 21 (FIG. 21) shows an annotated map of a fragment of a DNA sequence a hypoxia biosensor enhanced by amplification with COMET transcription factors and both subsequent positive feedback and positive feedback.

FIG. 22A (FIG. 22A) shows an annotated map of an HBS-CAR-ODD construct.

FIG. 22B (FIG. 22B) shows an annotated map of an HBS-CAR-ODD construct DNA sequence (SEQ ID NO: 19).

FIG. 23A (FIG. 23A) shows an annotated map of an EF1α-CAR construct.

FIG. 23B (FIG. 23B) shows an annotated map of an EF1α-CAR construct DNA sequence (SEQ ID NO: 20).

FIG. 24A (FIG. 24A) shows an annotated map of an HBS-CAR construct.

FIG. 24B (FIG. 24B) shows an annotated map of an HBS-CAR construct DNA sequence SEQ ID NO: 21 (sense strand) and SEQ ID NO: 39 (antisense strand).

FIG. 25A (FIG. 25A) shows an annotated map of an EF1α-CAR-ODD construct.

FIG. 25B (FIG. 25B) shows an annotated map of an EF1α-CAR-ODD construct DNA sequence SEQ ID NO: 22 (sense strand) and SEQ ID NO: 40 (antisense strand).

FIG. 26A (FIG. 26A) shows an annotated map of an EF1α-Null construct.

FIG. 26B (FIG. 26B) shows an annotated map of an EF1α-Null construct DNA sequence SEQ ID NO: 23 and 24 (sense strands) and SEQ ID NO: 41 and 42 (antisense strands).

FIG. 27A (FIG. 27A) shows an annotated map of an HBS-huHIF1α-T2A-CAR-ODD feedback construct.

FIG. 27B (FIG. 27B) shows an annotated map of an HBS-huHIF1α-T2A-CAR-ODD feedback construct DNA sequence SEQ ID NO: 25 (sense strand) and SEQ ID NO: 43 (antisense strand).

FIG. 28A (FIG. 28A) shows an annotated map of an HBS-CAR-ODD-T2A-huHIF1α feedback construct.

FIG. 28B (FIG. 28B) shows an annotated map of an HBS-CAR-ODD-T2A-huHIF1α feedback construct DNA sequence SEQ ID NO: 26 and 27 (sense strand) and SEQ ID NO: 44 and 45 (antisense strands).

FIG. 29A (FIG. 29A) shows an annotated map of an HBS-huHIF2α-T2A-CAR-ODD feedback construct.

FIG. 29B (FIG. 29B) shows an annotated map of an HBS-huHIF2α-T2A-CAR-ODD feedback construct DNA sequence SEQ ID NO: 28 (sense strand) and SEQ ID NO: 46 (antisense strand).

FIG. 30A (FIG. 30A) shows an annotated map of an HBS-CAR-ODD-T2A-huHIF2α feedback construct.

FIG. 30B (FIG. 30B) shows an annotated map of an HBS-CAR-ODD-T2A-huHIF2α feedback construct DNA sequence SEQ ID NO: 29 (sense strand) and SEQ ID NO: 47 (antisense strand).

FIG. 31A (FIG. 31A) shows an annotated map of a multiple transcriptional unit huHIF1α feedback construct.

FIG. 31B (FIG. 31B) shows an annotated map of a multiple transcriptional unit huHIF1a feedback construct DNA sequence SEQ ID NO: 30 (sense strand) and SEQ ID NO: 48 (antisense strand).

FIG. 32A (FIG. 32A) shows an annotated map of a multiple transcriptional unit huHIF2α feedback construct.

FIG. 32B (FIG. 32B) shows an annotated map of a multiple transcriptional unit huHIF2α feedback construct DNA sequence SEQ ID NO: 31 (sense strand) and SEQ ID NO: 49 (antisense strand).

FIG. 33 (FIG. 33) shows a quantification of open loop (no feedback) hypoxia biosensor construct (HBS) function in benchmarking experiments. Constructs depicted at left were introduced into HEK293FT cells via transposon-mediated integration. CAR expression was evaluated by flow cytometry after culturing cells at the indicated times under hypoxia (1% O₂, dashed lines) or normoxia (21% O₂, solid lines). Constructs including an engineered oxygen degradation domain (ODD) derived from Kosti et al. (2021, PMID 34401788, DOI: 10.1016/j.xpro.2021.100723) are also included. Constructs driven by the human EF1α promoter were included as constitutively transcribed controls. Data points represent n=3 biological replicates and error bars indicate standard deviation of the mean.

FIG. 34 (FIG. 34) shows a quantification of the function of hypoxia biosensor circuits employing co-cistronic huHIF1α feedback. Constructs depicted at left were introduced into HEK293FT cells via transposon-mediated integration. CAR expression was evaluated by flow cytometry after culturing cells at the indicated times under hypoxia (1% O₂, dashed lines) or normoxia (21% O₂, solid lines). These biosensors exhibited hypoxia-inducible expression, and expression levels are below those achieved with the baseline constitutive CAR-ODD configuration. Data points represent n=3 biological replicates and error bars indicate standard deviation. Benchmark data are the same as those depicted in FIG. 33.

FIG. 35 (FIG. 35) shows a quantification of the function of hypoxia biosensor circuits employing co-cistronic huHIF2α feedback. Constructs depicted at left were introduced into HEK293FT cells via transposon-mediated integration. CAR expression was evaluated by flow cytometry after culturing cells at the indicated times under hypoxia (1% O₂, dashed lines) or normoxia (21% O₂, solid lines). These novel biosensors exhibit no hypoxia-inducible expression, and expression levels are below those achieved with the baseline constitutive CAR-ODD configuration. Data points represent n=3 biological replicates and error bars indicate standard deviation. Benchmark data are the same as those depicted in FIG. 33.

FIG. 36 (FIG. 36) shows a quantification of the function of hypoxia biosensor circuits employing multiple transcriptional unit huHIF1α and huHIF2α feedback. Constructs depicted at left were introduced into HEK293FT cells via transposon-mediated integration. CAR expression was evaluated by flow cytometry after culturing cells at the indicated times under hypoxia (1% O₂, dashed lines) or normoxia (21% O₂, solid lines). These novel biosensors exhibit hypoxia-inducible expression for both huHIF1α and huHIF2α circuits, and for huHIF2α, expression levels exceed those achieved with the baseline constitutive CAR-ODD configuration. Data points represent n=3 biological replicates and error bars indicate standard deviation. Benchmark data are the same as those depicted in FIG. 33.

FIG. 37 (FIG. 37) shows a quantitative comparison across feedback circuit architectures. Data from FIGS. 34-36 are replotted on the same graph to enable direct evaluation of the impact of circuit architecture on performance. All data shown were collected in the same experiment. This comparison highlights the general observation that multiple transcriptional unit huHIF2α feedback mediates the greatest and most sustained level of CAR expression in response to hypoxia.

### DETAILED DESCRIPTION

The present disclosure provides novel DNA-based hypoxia biosensors (HBS). In some embodiments, provided herein are improved DNA-based hypoxia biosensors. The present disclosure encompasses several genetic circuits that enhance DNA-based hypoxia biosensors. One class of genetic circuits relies on positive feedback with endogenous Hypoxia Inducible Factor (HIF) proteins. This strategy is founded in the unexpected and surprising result from experiments that demonstrated that expression of mutant HIF1α containing mutations that made it resistant to oxygen-induced degradation drives more reporter expression from the biosensor than does wild-type HIF1α under hypoxic conditions in cell culture and under conditions including treatment with the hypoxia mimetic cobalt.

A second class of genetic circuits employs COMET (Composable Mammalian Elements of Transcription) transcription factors to enhance the sensitivity of a hypoxia biosensor. These circuits can enhance the hypoxia biosensor by increasing both the maximum magnitude of the gene expression in the presence of hypoxia and the speed with which it reaches this maximum. These circuits advantageously achieve amplification of a biosensor's response to hypoxia without amplifying the effects upon natural targets of HIF1α or HIF2α. Moreover, these circuits present the advantage of amplification without the need for co-factors that mediate the effects of HIF2α (e.g., HAF).

The present disclosure further provides a DNA-based hypoxia biosensor, comprising: (a) a hypoxia-inducible promoter; (b) a nucleic acid sequence encoding a functional element; and (c) at least one nucleic acid sequence encoding a feedback element. In some aspects, a hypoxia biosensor of provided herein comprises a hypoxia-inducible promoter comprising (i) an Egr-1-binding site (EBS) from a Egr-1 gene, a metal-response element (MRE) from a metallothionein gene, a hypoxia-response element (HRE), or a combination thereof, and (ii) a minimal promoter. In some embodiments, a hypoxia-inducible promoter comprises (i) an Egr-1-binding site (EBS) from an Egr-1 gene, a metal-response element (MRE) from a metallothionein gene, and at least one a hypoxia-response element (HRE), and (ii) a minimal promoter. In some aspects, the at least one HRE may be or comprise three HREs (e.g., EBS-MRE-3×HRE).

In some embodiments, a feedback element is a positive feedback element. In some aspects, a positive feedback element of a hypoxia biosensor is selected from HIF1α and HIF2α. In some embodiments, a hypoxia biosensor provided herein may include a nucleic acid sequence encoding a first feedback element selected from HIF1α and HIF2α, and a nucleic acid sequence encoding a second feedback element, wherein the second feedback element is, optionally, HIF1β.

The present disclosure further provides a DNA-based hypoxia biosensor, comprising: (a) a hypoxia-inducible promoter; (b) a nucleic acid sequence encoding a functional element; (c) at least one nucleic acid sequence encoding one or more engineered proteins selected from the group consisting of: (i) an engineered protein that activates gene expression, wherein the engineered protein comprises a DNA binding domain, and a transcription activator domain; (ii) an engineered protein that inhibits gene expression, (iii) an engineered protein that inhibits gene expression, the engineered protein comprising one or more of a DNA binding domain, a bulky domain, a chromatin remodeling domain, and a transcription inhibitor domain; and (iv) a combination of two engineered proteins comprising a first engineered protein comprising a DNA binding domain fused to a dimerization domain, and a second engineered protein comprising a transcription regulator domain fused to a dimerization domain, wherein the dimerization domains of the two engineered proteins dimerize in the presence of a stimulus to which the dimerization domains of the two engineered proteins bind; and (d) one or more DNA binding sites for the DNA binding domain of the engineered protein(s) of (c).

A hypoxia biosensor provided herein may include a minimal promoter. A minimal promoter may be any minimal promoter known in the art. Non-limiting examples of minimal promoters include simian virus 40 (SV40) promoter, cytomegalovirus (CMV) promoter, and a synthetic promoter. A non-limiting example of a synthetic promoter is a YB_TATA promoter. In some embodiments, a reporter is selected from a fluorophore, a luciferase, a peroxidase, and any combination thereof.

A hypoxia biosensor of the present disclosure may comprise an engineered protein comprising at least one oxygen-degradation domain (ODD). A hypoxia biosensor of the present disclosure may comprise a functional element comprising at least one oxygen-degradation domain (ODD).

Hypoxia biosensors of the present disclosure may be arranged in various ways, as explained in more detail below. In some embodiments, a hypoxia biosensor may be arranged such that at least one DNA binding site for a DNA binding domain of an engineered protein(s) is 5′ of a nucleic acid sequence encoding the functional element. In some aspects, a hypoxia biosensor may be arranged such that at least one DNA binding site for a DNA binding domain of an engineered protein(s) is 5′ of a hypoxia-inducible promoter. In some embodiments, a hypoxia biosensor may be arranged such that at least one DNA binding site for a DNA binding domain of an engineered protein(s) is 5′ of a nucleic acid sequence encoding a functional element, and at least one DNA binding site for a DNA binding domain of an engineered protein(s) is 5′ of a hypoxia-inducible promoter. In some embodiments, a hypoxia biosensor may be arranged such that at least one DNA binding site for a DNA binding domain of an engineered protein(s) is 5′ of at least one nucleic acid sequence encoding the one or more engineered proteins. In some aspects, hypoxia biosensor may be arranged such that at least one DNA binding site for the DNA binding domain of an engineered protein(s) is 5′ of a nucleic acid sequence encoding a functional element; at least one DNA binding site for the DNA binding domain of the engineered protein(s) is 5′ of a hypoxia-inducible promoter; and at least one DNA binding site for the DNA binding domain of the engineered protein(s) is 5′ of at least one nucleic acid sequence encoding the one or more engineered proteins. In some embodiments, a hypoxia biosensor comprises at least one DNA binding site for the DNA binding domain of the engineered protein(s) is 5′ of the nucleic acid sequence encoding the functional element, and at least one DNA binding site for the DNA binding domain of the engineered protein(s) is 5′ of at least one nucleic acid sequence encoding the one or more engineered proteins. In some embodiments, a hypoxia biosensor comprises at least two nucleic acid sequences encoding one or more engineered proteins, and, optionally, wherein there is at least one DNA binding site for a DNA binding domain of the engineered protein(s) that is 5′ to each of the at least two nucleic acid sequences encoding one or more engineered proteins.

In some embodiments, a hypoxia biosensor comprises an engineered protein(s) comprising at least one split intein on the C-terminus or N-terminus of the DNA binding domain and/or the transcription activator domain.

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a peptide” includes a single peptide as well as a plurality of peptides, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding polynucleotide and amino acid sequences, “variant,” “mutant,” or “derivative” may be defined as a sequence having at least 50% sequence identity to the particular sequence over a certain length of one of the sequences using blastn or blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of variant, mutant, or derivative sequences may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode the same or similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

“Transformation” or “transfection” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide. Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g., the fusion protein disclosed herein).

Certain proteins or polypeptide sequences disclosed herein (e.g., split inteins) may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

The term “functional element” refers to a molecule, or a nucleic acid sequence encoding a molecule, that can be used to perform a given cellular or subcellular function or process. For example, a functional element may include a nucleic acid sequence encoding a protein-coding or non-coding gene sequence or fragment thereof, wherein expression of the functional element (e.g., said gene sequence) results in the production of a target molecule (e.g., a target polypeptide) by a cell in which the functional element is expressed. Non-limiting examples of functional element target molecules include therapeutic proteins (e.g., cytokines, antibodies, chimeric antigen receptors (CARs), etc.), mediators of downstream regulation of gene expression (e.g., transcription factors, synthetic transcription factors, transcriptional activators or repressors, dCas9-based transcriptional activators, repressors, base editors, prime editors, etc.), and reporters. In some embodiments, a functional element comprises a chimeric antigen receptor (CAR). A functional element may also include, for example, a nucleic acid sequence encoding a regulatory RNA molecule, such as a microRNA (miRNA), long non-coding RNA (lncRNA), short interfering RNA (siRNA). A regulatory RNA molecule may regulate ribozyme activity, the induction of innate immunity, or other cellular or subcellular functions. In some embodiments described herein, a functional element may be or comprise a reporter element.

The term “reporter” or “reporter element” refers to a molecule, or a nucleic acid sequence encoding a molecule, that can be used as an indicator of the occurrence or level of a particular biological process, activity, event, or state in a cell or organism. Reporters typically have one or more properties or enzymatic activities that allow them to be readily measured or that allow detection of a cell that expresses the reporter molecule. In general, a cell can be assayed for the presence of a reporter molecule by measuring the reporter molecule itself or an enzymatic activity of the reporter protein. Detectable characteristics or activities that a reporter may have include, e.g., fluorescence, bioluminescence, ability to catalyze a reaction that produces a fluorescent or colored substance in the presence of a suitable substrate, or other readouts based on emission and/or absorption of photons (light). Typically, a reporter molecule is a molecule that is not endogenously expressed by a cell or organism in which the reporter molecule is used.

### II. Abbreviations

| | |
|---|---|
| AD | Activation domain |
| CMV | Cytomegalovirus promoter |
| COMET | COmposable Mammalian Elements of Transcription |
| DMSO | Dimethyl sulfoxide vehicle for rapamycin treatment |
| DsRed | Abbreviation used for DsRed-Express2 |
| MEFL | Molecules of Equivalent Fluorescein |
| MEPTR | Molecules of Equivalent PE-Texas Red |
| MFI | Mean fluorescence intensity |
| TF | Transcription factor |
| VP16 | VP16 activation domain |
| VP64 | VP64 activation domain |
| ZF | Zinc finger |
| ZFa | Zinc finger activator |

### III. Engineered Genetic Circuits for Hypoxia Sensing

The technical field of the disclosed platform technology relates to biological engineering in mammalian synthetic biology. Mammalian cells can be programmed for numerous applications, ranging from customized cell-based therapeutics to tools for probing fundamental biological questions.

Recently, the Composable Mammalian Elements of Transcription (COMET) toolkit was developed for engineering genetic programs in mammalian cells (Donahue et al., Nat Commun 11, 779 (2020)). In the Examples provided herein, several HBS designs and their response to various levels and durations of hypoxia were evaluated in two cell types—the chassis HEK293FT humanoid cell line and the B16F10 model line for murine melanoma. These studies were performed using the landing pad (LP) system, which was developed for the rapid prototyping of biosensors in a genomic context. An LP is a targeted integration locus, pre-engineered in a safe harbor locus, in which large amounts of DNA can be readily inserted using a transposase. LPs have several advantages over other methodologies, such as lentiviral transduction, including a much higher limit on cargo size. Additionally, as the cells with cargo integrated into the landing pad locus are genetically identical after integration, this methodology removes the confounding factor of biosensor integration locus and makes the resulting population more homogenous. During initial evaluation of the HBS designs in the LP context, several opportunities for improving biosensor performance by modulating the signal with genetic circuits, including those based on endogenous and synthetic TFs, were discovered. Several such circuits were designed, tested in vitro. These efforts will ultimately result in HBSs that are robust to the dysregulation of the hypoxic response and useful for fundamental and translational research, diagnostics, and therapeutics.

### Genetic Circuits Employing Positive Feedback of Endogenous HIF Proteins

Genetic circuits provided herein form the foundation of improved hypoxia biosensors.

In some embodiments, a DNA-based hypoxia biosensor (HBS) may comprise a hypoxia-inducible promoter. Any hypoxia-inducible promoter may be used in accordance with the present disclosure. Non-limiting examples of hypoxia-inducible promoters include promoters comprising one or more of (i) an Egr-1-binding site (EBS) from a Egr-1 gene, a metal-response element (MRE) from a metallothionein gene, at least one (e.g., at least 1, at least 2, at least 3, or at least 4 or more) hypoxia-response element (HRE), or a combination thereof. A hypoxia-inducible promoter may comprise a minimal promoter. Any minimal promoter may be used in accordance with the present disclosure. Non-limiting examples of minimal promoters include those from simian virus 40 (SV40), cytomegalovirus (CMV), and a synthetic promoter. Synthetic promoters that may be used in accordance with the present disclosure include but are not limited to YB_TATA.

In some embodiments, a DNA-based hypoxia biosensor (HBS) may comprise a nucleic acid sequence encoding a functional element. A functional element can be, without limitation, a gene sequence or fragment thereof, a nucleic acid sequence encoding a regulatory RNA molecule, or a reporter element. A reporter or reporter element is a molecule, or a nucleic acid sequence encoding a molecule, that can be used as an indicator of the occurrence or level of a particular biological process, activity, event, or state in a cell or organism (e.g., hypoxia). A reporter or reporter element may indicate a cellular state through any known mechanism. A reporter element may be operably linked to a promoter. Non-limiting examples of reporters and reporter elements include a fluorophore, a luciferase, a peroxidase, or a combination thereof. In some embodiments, a combination may include a fusion protein, such as a fusion protein comprising a luciferase polypeptide fused to a fluorophore. Such a combination or fusion protein is useful in generating a biosensor, such as a biosensor that utilizes bioluminescence resonance energy transfer. Non-limiting examples of fluorophores include DsRed, DsRed-Express2, GFP, mScarlet-I, LumiLuc, LumiScarlet, and mNeonGreen. Any fluorophore, luciferase, or peroxidase may be used in accordance with the present disclosure. In some embodiments, a functional element may comprise at least one (e.g., 1, 2, 3, 4, 5, or more) oxygen-degradation domain (ODD). Appending an oxygen-degradation domain to functional element described herein is one strategy to reduce any potential amplification of leaky gene expression or constitutive activation of positive feedback circuits. Such an ODD may serve to render the functional element oxygen-instable (i.e., degraded in the presence of oxygen) (also referred to as oxygen-sensitive). An ODD may be an amino acid motif from a HIF protein (e.g., HIF1α). An ODD may be placed on the N-terminal region, the C-terminal region, an internal linker, or another region of a functional element.

Hypoxia biosensors of the present disclosure may comprise a feedback element, such as a positive feedback element. In some embodiments, a DNA-based hypoxia biosensor (HBS) may comprise at least one nucleic acid sequence encoding a feedback element (e.g., a positive feedback element). A positive feedback element is an element which, upon activation of the biosensor, facilitates additional activation of the biosensor through direct or indirect means. For example, a hypoxia biosensor that is activatable by the binding of a HIF protein may comprise a feedback element, which may be or comprise a nucleic acid sequence encoding a HIF protein. In such a hypoxia biosensor, activation of the biosensor by the binding of HIF protein leads to production of a HIF protein, which may subsequently activate the hypoxia biosensor in a positive feedback loop. Positive feedback elements may be any positive feedback element that permit hypoxia sensing by positive feedback. Non-limiting examples of positive feedback elements include a nucleic acid sequence encoding HIF1α and a nucleic acid sequence encoding HIF2α.

In some embodiments, a hypoxia biosensor may comprise one or more (e.g., one, two, three, or more) feedback elements. In some embodiments, one or more feedback elements may be a positive feedback element. In some embodiments, one or more feedback elements may be a negative feedback element. In some embodiments, a hypoxia biosensor may comprise a first positive feedback element and a second positive feedback element. In some embodiments, a hypoxia biosensor may comprise a nucleic acid sequence encoding a first positive feedback element selected from HIF1α and HIF2α, and a nucleic acid sequence encoding a second positive feedback element, wherein the second positive feedback element is, optionally, HIF1β.

In some embodiments, a DNA-based hypoxia biosensor (HBS) may comprise (a) a hypoxia-inducible promoter; (b) a nucleic acid sequence encoding a functional element; and (c) at least one nucleic acid sequence encoding a feedback element. In some embodiments, a feedback element may be a positive feedback element.

In some embodiments, a DNA-based hypoxia biosensor (HBS) may include a nucleic acid sequence encoding a cleavage peptide. In some embodiments, the cleavage peptide is a self-cleaving T2A peptide.

In some embodiments, a DNA-based hypoxia biosensor (HBS) may comprise (a) a hypoxia-inducible promoter; (b) a nucleic acid sequence encoding a functional element; and (c) at least one nucleic acid sequence encoding a feedback element, wherein the nucleic acid sequence encoding a functional element and the at least one nucleic acid sequence encoding a feedback element are separated by a nucleic acid sequence encoding a cleavage peptide, optionally wherein the cleavage peptide is a self-cleaving peptide, such as a T2A peptide.

In some embodiments of a DNA-based hypoxia biosensor described herein, the nucleic acid sequence encoding a functional element is 5′ of the at least one nucleic acid sequence encoding a feedback element. In some embodiments of a DNA-based hypoxia biosensor described herein, the nucleic acid sequence encoding a functional element is 3′ of the at least one nucleic acid sequence encoding a feedback element.

In some embodiments of a DNA-based hypoxia biosensor described herein, the hypoxia biosensor comprises at least two (e.g., 2, 3, 4, 5, or more) hypoxia-inducible promoters. In some embodiments, the nucleic acid sequence encoding a functional element and the at least one nucleic acid sequence encoding a feedback element are operably linked to separate hypoxia-inducible promoters. In some embodiments, the separate hypoxia-inducible promoters comprise the same nucleotide or protein sequence, but are present in physically distinct locations within the DNA-based hypoxia biosensor. In some embodiments, the separate hypoxia-inducible promoters comprise different nucleotide or protein sequences, but are present in physically distinct locations within the DNA-based hypoxia biosensor.

### Genetic Circuits Employing COMET Transcription Factors

Genetic circuits of the present disclosure may employ synthetic transcription factors, and/or systems employing the same. Genetic circuits provided herein may employ Composable Mammalian Elements of Transcription (COMET) transcription factors. The COMET platform and some COMET transcription factors are described in WO2018/175865A1, Donahue et al., Nat. Comm. 11; 79 (2020), and PCT/US2021/050584, each of which is hereby incorporated herein by reference in its entirety.

In some embodiments, a DNA-based hypoxia biosensor may comprise at least one (e.g., 1, 2, 3, 4, or 5 or more) nucleic acid sequence encoding one or more engineered proteins. In some embodiments, engineered proteins may comprise at least two functional domains: (i) a DNA binding domain, (ii) a transcription modulation domain, which may activate or inhibit transcription of the hypoxia biosensor. The engineered proteins of the present disclosure may comprise at least one or one or more (e.g., 1, 2, 3, 4, 5, or more) split inteins. Split inteins are short peptide elements comprising complementary domains that fold and trans-splice to covalently ligate flanking domains. Accordingly, the incorporation of split inteins into the one or more engineered proteins allows for post-translational modification of the engineered proteins in response to stimuli (e.g., hypoxia) from the system in which the genetic circuit is employed.

In some embodiments, the DNA binding domain of an engineered protein may comprise, for example, all of or a functional fragment of a zinc finger domain, such as ZF1, ZF2, ZF3, ZF4, ZF5, ZF6, ZF7, ZF8, ZF9, ZF10, ZF11, ZF12, ZF13, ZF14, or ZF15. In some embodiments, the DNA binding domain of an engineered protein may comprise, for example, all of or a functional fragment of a zinc finger protein comprising more than three DNA-binding domains, other classes of programmable DNA binding domains (e.g., transcription activator-like effector (TALE)), DNA binding domains derived from microbial proteins (e.g., tetR, lacI, etc.), and/or Cas9 or variants of Cas9 and other Cas proteins, including catalytically inactive variants (e.g., dCas9).

In some embodiments, a transcription activator domain of an engineered protein may comprise, for example, all of or a functional fragment of Herpes simplex virus protein 16 (VP16), a synthetic tetramer of VP16 (VP64), nuclear factor (NF) kappa-B (p65) or a subunit thereof, heat shock transcription factor 1 (HSF1), replication and transcription activator (RTA) of the gamma-herpesvirus family, p53, acidic domains (also known as “acid blobs” or “negative noodles,” rich in D and E amino acids, present in Gal4, Gcn4 and VP16), glutamine-rich domains (which may comprise multiple repetitions like “QQQXXXQQQ,” (SEQ ID NO: 11) like those present in transcription factor Sp1), proline-rich domains (which may comprise repetitions like “PPPXXXPPP,” (SEQ ID NO: 12) like those present in c-jun, AP2, and Oct-2), isoleucine-rich domains (which may comprise repetitions of “IIXXII,” (SEQ ID NO: 13) like those present in NTF-1), and/or multipartite activators, such as VP64-p65-Rta (i.e., “VPR”; see Chavez et al., Nat Methods. 2015 April; 12 (4): 326-328).

In some embodiments, a transcription inhibition/inhibitor domain of an engineered protein may comprise, for example, all of or a functional fragment of ZF, KRAB, Polycomb complexes, any domain that can fulfill a similar function for inhibition as a bulky DsRed variant, any domain which sterically occludes recruitment of the RNA polymerase complex or accessory factors, and/or chromatin modification modalities including histone de-acetylation, histone methylation, etc. as reviewed in Beisel & Paro, Nature Reviews Genetics, 12:123-135 (2011).

In some embodiments, a split intein (or one or more split inteins) may be incorporated into an engineered protein between the DNA binding domain and the transcription modulation domain (e.g., transcription activation domain or transcription inhibition domain). In some embodiments, a split intein (or one or more split inteins) may be incorporated onto the N-terminus of an engineered protein. In some embodiments, a split intein (or one or more split inteins) may be incorporated onto the C-terminus of an engineered protein. In some embodiments, a split intein (or one or more split inteins) may be incorporated onto both the N-terminus and the C-terminus of an engineered protein.

Many split inteins are known in the art, including but not limited to, gp41-1, Npu DnaE, Ssp DnaE, Mtu RecA, Sce VMA, Ssp DnaB-S0, Ssp DnaB-S1, and Ssp GyrB-S11.

In some embodiments, a DNA-based hypoxia biosensor may comprise one or more DNA binding sites for the DNA binding domain of one or more engineered proteins. In general, the DNA binding domain of the one or more engineered proteins is capable of binding to a DNA binding site on the hypoxia biosensor.

In some embodiments, a DNA-based hypoxia biosensor may comprise (a) a hypoxia-inducible promoter; (b) a nucleic acid sequence encoding a functional element; (c) at least one (e.g., 1, 2, 3, 4, or 5 or more) nucleic acid sequence encoding one or more engineered proteins; and (d) one or more DNA binding sites for the DNA binding domain of the engineered protein(s) of (c).

In some embodiments, a hypoxia biosensor may comprise a minimal promoter and a gene of interest, such as a reporter gene of some kind (e.g., a fluorescent protein or another detectable protein/peptide/signal). A minimal promoter may be from any applicable promoter, including but not limited to simian virus 40 (SV40), cytomegalovirus (CMV), or a synthetic promoter, as described supra (e.g., YB_TATA).

In some embodiments, a DNA-based hypoxia biosensor may comprise at least one nucleic acid sequence encoding one or more engineered proteins selected from the group consisting of: (i) an engineered protein that activates gene expression, wherein the engineered protein comprises a DNA binding domain and a transcription activator domain; (ii) an engineered protein that inhibits gene expression, the engineered protein comprising a DNA binding domain and a transcription inhibitor domain; and (iii) a combination of two engineered proteins comprising a first engineered protein comprising a DNA binding domain fused to a dimerization domain, and a second engineered protein comprising a transcription regulator domain fused to a dimerization domain, wherein the dimerization domains of the two engineered proteins dimerize in the presence of a stimulus to which the dimerization domains of the two engineered proteins bind.

In some embodiments, a DNA-based hypoxia biosensor may comprise at least one nucleic acid sequence encoding one or more engineered proteins selected from the group consisting of: (i) an engineered protein that activates gene expression, wherein the engineered protein comprises a DNA binding domain, a transcription activator domain, and at least one split intein on the C-terminus or N-terminus of the DNA binding domain and/or the transcription activator domain; (ii) an engineered protein that inhibits gene expression, the engineered protein comprising a DNA binding domain, a transcription inhibitor domain, and at least one split intein on the C-terminus or N-terminus of the DNA binding domain and/or the transcription inhibitor domain; and (iii) a combination of two engineered proteins comprising a first engineered protein comprising a DNA binding domain fused to a dimerization domain, and a second engineered protein comprising a transcription regulator domain fused to a dimerization domain, wherein the dimerization domains of the two engineered proteins dimerize in the presence of a stimulus to which the dimerization domains of the two engineered proteins bind, and wherein and the first engineered protein and the second engineered protein each comprise at least one split intein.

In some embodiments, an engineered protein may comprise at least one (e.g., 1, 2, 3, 4, 5, or more) oxygen-degradation domain (ODD). Appending an oxygen-degradation domain to an engineered protein described herein is one strategy to reduce any potential amplification of leaky gene expression or constitutive activation of positive feedback circuits. Such an ODD may serve to render the engineered protein oxygen-instable (i.e., degraded in the presence of oxygen) (also referred to as oxygen-sensitive). An ODD may be an amino acid motif from a HIF protein (e.g., HIF1α). An ODD may be placed on the N-terminal region, the C-terminal region, an internal linker, or another region of an engineered protein.

In some embodiments, a DNA-based hypoxia biosensor may comprise at least one DNA binding site for the DNA binding domain of the engineered protein(s). In some embodiments, the DNA binding site may be positioned 5′ of a nucleic acid sequence encoding a functional element. In some embodiments, the DNA binding site for the DNA binding domain of the engineered protein(s) may be positioned 5′ of the hypoxia-inducible promoter. In some embodiments, at least one DNA binding site for the DNA binding domain of the engineered protein(s) may be positioned 5′ of the nucleic acid sequence encoding the functional element, and at least one DNA binding site for the DNA binding domain of the engineered protein(s) may be positioned 5′ of the hypoxia-inducible promoter. In some embodiments, at least one DNA binding site for the DNA binding domain of the engineered protein(s) may be positioned 5′ of at least one nucleic acid sequence encoding the one or more engineered proteins. In some embodiments, at least one DNA binding site for the DNA binding domain of the engineered protein(s) may be positioned 5′ of the nucleic acid sequence encoding the functional element; at least one DNA binding site for the DNA binding domain of the engineered protein(s) may be positioned 5′ of the hypoxia-inducible promoter; and at least one DNA binding site for the DNA binding domain of the engineered protein(s) may be positioned 5′ of at least one nucleic acid sequence encoding the one or more engineered proteins. In some embodiments, a hypoxia biosensor may include at least two nucleic acid sequences encoding one or more engineered proteins, and, optionally, wherein there is at least one DNA binding site for the DNA binding domain of the engineered protein(s) that is 5′ to each of the at least two nucleic acid sequences encoding one or more engineered proteins.

### IV. Methods of Using the Disclosed Engineered Circuits

The present disclosure provides methods in which a host cell may be transiently or non-transiently transfected (i.e., stably transfected) with one or more hypoxia biosensors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). Suitable cells may include HEK293 cells, such as HEK293FT cells. Suitable cells may include cancer cells. Suitable cells may include cells of a cancer-derived cell line, such as B16F10 cells. A cell transfected with one or more hypoxia biosensors described herein may be used to establish a new cell line comprising one or more HBS sequences. In the methods contemplated herein, a cell may be transiently transfected with the components of a system as described herein (such as by transient transfection of one or more vectors or plasmids, or transfection with RNA), and modified through the activity of a complex, in order to establish a new cell line comprising cells containing the modification but lacking any other exogenous sequence.

The presently disclosed methods may include delivering one or more polynucleotides, such as or one or more vectors as described herein and/or one or proteins transcribed therefrom, to a host cell. Further contemplated are host cells produced by such methods, and organisms (such as animals, plants, or fungi) comprising or produced from such cells. Conventional viral and non-viral based gene transfer methods can be used to introduce nucleic acids in mammalian cells or target tissues. Non-viral vector delivery systems include DNA plasmids, RNA (e.g. a transcript of a vector described herein), naked nucleic acid, nucleic acid complexed with a delivery vehicle (such as a liposome), and transposons. Viral vector delivery systems include DNA and RNA viruses, which have either episomal or integrated genomes after delivery to the cell.

### V. Applications and Advantages of the Disclosed HBSs

In one aspect, the disclosed compositions can be used in methods comprising mammalian cell-based therapies for treating diverse diseases including cancer, autoimmune disease, vascular disease, cardiac disease, metabolic diseases, and ischemic injury to tissues including the brain, kidney, and heart. Cell based therapies have been successful in treating many hematologic malignancies, leading to substantial increases in survival for many patients. However, translating these successes to the treatment of solid tumors has been difficult for many reasons. Among these is the challenge of discovering tumor specific antigens and developing biosensors against these targets. While this approach often leads to tumor-specific therapeutics, the diversity of antigens across cancers results in only incremental progress with each new therapeutic. An alternative approach is to develop biosensors against features of the tumor environment common across many malignancies, for example, hypoxia. Such biosensors have already been useful in restricting chimeric antigen receptor (CAR) expression to the tumor microenvironment. Further, biosensors intended for these therapeutic purposes will provide utility beyond this original design. For instance, a hypoxia biosensor may be used, via in vivo imaging, to study the development of hypoxia in a tumor longitudinally or in response to treatment. Some of the genetic circuits described herein rely on positive feedback loops utilizing the endogenous Hypoxia Inducible Factor genes. These enhancements result in hypoxia biosensors that output higher levels of gene expression more rapidly than the original biosensor design. Other genetic circuits described herein rely on amplification and positive feedback circuits utilizing transcription factors from the Composable Mammalian Elements of Transcription (COMET), including several that are destabilized in the presence of oxygen, resulting in increased specificity of the biosensor for hypoxic conditions. Ultimately, a high-performing hypoxia biosensor could be used, for example, for discovery, diagnostics, monitoring, and therapeutics.

In another aspect, the disclosed compositions and methods can be applied to methods of biomanufacturing using cells engineered to perform sophisticated function, including the detection of hypoxia. In another aspect, the disclosed compositions and methods can be applied to methods of gene therapy comprising delivery of compact genetic programs using parts/strategies described in this disclosure. In another aspect, the disclosed compositions and methods can be applied to methods of preparing stem cell-based products (e.g., for therapy or research) in which differentiation is controlled by a genetic program built using this technology platform.

Further applications of the disclosed technology platform may include, but are not limited to: (i) engineered cell-based therapies for cancer, autoimmune disease, vascular disease, cardiac disease, metabolic diseases, ischemic injury to tissues including the brain, kidney, and heart; regenerative medicine, and many other diseases; (ii) investigating fundamental biological questions (research), for example by expressing transgenes in mammalian cells at various levels or only under certain conditions; and (c) control of gene expression in biotechnology, for example production of recombinant proteins in mammalian cells.

Further advantages of the disclosed technology platform may include, but are not limited to: (i) the disclosed technology comprises highly sensitive and specific hypoxia biosensors employing a variety of novel genetic circuits based on signal amplification and feedback; (ii) many different parameters are readily tunable in the disclosed technology using either design-driven or experimentally identified variations in the engineered proteins and/or DNA sequences of the disclosed technology; (iii) some implementations of the proposed technology enable amplification of biosensor output without amplification of the effects upon natural targets of endogenous hypoxia-responsive proteins.

Altogether, the present disclosure greatly expands the mammalian genetic program design space, especially as they apply to the design of biosensors (e.g., hypoxia biosensors) for mammalian cells.

The following examples are given to illustrate the disclosed hypoxia biosensors. It should be understood, however, that the invention is not to be limited to the specific conditions or details described in these examples.

### EXAMPLES

### Example 1—Materials and Methods

### Experimental Method Details

### Cloning, Plasmid, and Oligonucleotide Sources

All constructs were initially characterized in the pPD005 backbone, which is a version of pcDNA3.1 (+), modified as described previously (Donahue et al., Nat Commun 11, 779 (2020)). All HBS components were transferred into the mMoClo system²³, with Leonard Lab modifications, as described previously (Donahue et al., Nat Commun 11, 779 (2020)). Coding sequences are generally flanked by NheI and NotI restriction sites. Promoter regions are generally flanked by BglII or MluI on the 5′ end and NheI on the 3′ end. The HBS was synthesized with overlapping oligonucleotides from a previously published study (Lee et al., Gene Ther 13, 857-868 (2006)), as were the YB_TATA and CMV_min promoters (Ede et al., ACS Synth Biol 5, 395-404 (2016)). The SV40_min was PCR amplified prior to use (Ede et al., ACS Synth Biol 5, 395- 404 (2016)). The stable HIF1α mutant was sourced from pcDNA3 mHIF-1α MYC (P402A/P577A/N813A) (Addgene #44028). EF1α and TetON3G were sourced from pLVX-Tet3G (Clontech), and TRE3GV was sourced from pLVX-TRE3G (Clontech). BlastR was sourced from lenti dCAS-VP64_Blast (Addgene #61425). The CHS4 insulator was sourced from PhiC31-Neo-ins-5×TetO-pEF-H2B-Citrin-ins (Addgene #78099). DsRed-Express2 was obtained by site directed mutagenesis of pDsRed2-N1, and an internal BpiI restriction site in the coding region was ablated by making a sense mutation with site directed mutagenesis. EBFP2 was sourced from pEBFP2-Nuc (Addgene #14893 addgene.org/14893/). EYFP, was sourced from plasmids we previously described (Addgene #58855). mTagBFP2 (Subach et al., PLOS One 6, e28674 (2011)), mNeonGreen (Shaner et al., Nat Methods 10, 407-409 (2013)), mRuby3 (Bajar et al., Sci Rep 6, 20889 (2016)), miRFP670 (Shemetov et al., Cell Chem Biol 24, 758-766.e753 (2017); Oliinyk et al., Nat Commun 10, 279 (2019)), and miRFP720 (Shcherbakova et al., Nat Chem Biol 14, 591-600 (2018)) were synthesized as Gene Strings by ThermoFisher.

### Plasmid Preparation

TOP10 E. coli were grown overnight in 100 mL of LB with the appropriate selective antibiotic. The following morning, cells were pelleted at 3000×g for 10 min and then resuspended in 4 mL of a solution of 25 mM Tris pH 8.0, 10 mM EDTA, and 15% sucrose. Cells were lysed for 15 min by addition of 8 mL of a solution of 0.2 M NaOH and 1% SDS, followed by neutralization with 5 mL of 3 M sodium acetate (pH 5.2). Precipitate was pelleted by centrifugation at 9000×g for 20 min. Supernatant was decanted and treated with RNAse A for 1 h at 37° C. 5 mL of phenol chloroform was added, and the solution was mixed and then centrifuged at 7500×g for 20 min. The aqueous layer was removed and subjected to another round of phenol chloroform extraction with 7 mL of phenol chloroform. The aqueous layer was then subjected to an isopropanol precipitation (41% final volume isopropanol, 10 min at room temperature, 9000×g for 20 min), and the pellet was briefly dried and resuspended in 420 μL of water. The DNA mixture was incubated on ice for at least 12 h in a solution of 6.5% PEG 20,000 and 0.4 M NaCl (1 mL final volume). DNA was precipitated with centrifugation at maximum speed for 20 min. The pellet was washed once with ethanol, dried for several h at 37° C., and resuspended for several h in TE buffer (10 mM Tris, 1 mM EDTA, pH 8.0). DNA purity and concentration were confirmed using a Nanodrop 2000 (Thermo Fisher).

### Mammalian Cell Culture

HEK293FT cells were cultured in complete DMEM medium containing 1% DMEM powder (Gibco #31600091), 0.35% w/v D-glucose (Sigma #50-99-7), 0.37% w/v sodium bicarbonate (Fisher #S233-500), 10% heat-inactivated FBS (Gibco #16140071), 4 mM L-glutamine (Gibco #25030081), and 100 U ml⁻¹ penicillin and 100 μg ml⁻¹ streptomycin (Gibco #15140122) in tissue culture-treated 10 cm dishes (Corning #500001672) at 37° C. in 5% CO₂. To passage, medium was aspirated, and cells were washed in PBS, incubated in trypsin-EDTA (Gibco #25300054; 37° C., 5 min), detached by tapping the dish, and resuspended in fresh medium and plated. This cell line tested negative for Mycoplasma using the MycoAlert Mycoplasma detection kit (Lonza #LT07-318).

B16F10 cells were cultured in DMEM (Gibco #31600-091) with 4.5 g/L glucose (1 g/L, Gibco #31600-091; 3.5 g/L additional, Sigma #G7021), 3.7 g/L sodium bicarbonate (Fisher Scientific #S233), 10% FBS (Gibco #16140-071), 6 mM L-glutamine (2 mM, Gibco #31600-091; 4 mM additional, Gibco #25030-081), penicillin (100 U/μL), and streptomycin (100 μg/mL) (Gibco #15140122), in a 37° C. incubator with 5% CO₂. Cells were subcultured at a 1:10 to 1:20 ratio every 2-3 d using Trypsin-EDTA (Gibco #25300-054).

### Transfection

Transient transfection of HEK293FT cells was conducted using the calcium phosphate methodology. Cells were plated at a minimum density of 1.5×10⁵ cells per well in a 24-well plate in 0.5 mL DMEM, supplemented as described above. For surface staining experiments, cells were plated at a minimum density of 3.0×10⁵ cells per well in a 12-well plate in 1 mL DMEM. After at least 6 h, by which time the cells had adhered to the plate, the cells were transfected. For transfection, plasmids were mixed in H₂O, and 2 M CaCl₂) was added to a final concentration of 0.3 M CaCl₂). This mixture was added dropwise to an equal-volume solution of 2×HEPES-Buffered Saline (280 mM NaCl, 0.5 M HEPES, 1.5 mM Na₂HPO₄) and gently pipetted up and down four times. After 2.5-4 min, the solution was mixed vigorously by pipetting ten times. 100 μL of this mixture was added dropwise to the plated cells in 24-well plates, 200 μL was added to the plated cells in 12-well plates, or 400 μL was added to the plated cells in 6-well plates, and the plates were gently swirled. The next morning, the medium was aspirated and replaced with fresh medium. In some assays, fresh medium contained cobalt (ii) chloride, as described in the figures. Typically, at 36-48 h post-transfection and at least 24 h post-media change, cells were harvested.

In some experiments, HEK293FT cells were transiently transfected via lipofection. From exponentially growing HEK293LP cells, 1.0×10⁵ cells were plated per well (1.0 mL medium) in 12-well format, and cells were cultured for 24 h to allow cells to attach and spread. When cells reached 50-75% confluence, plasmids were transfected by lipofection with Lipofectamine LTX with PLUS Reagent (ThermoFisher 15338100). Plasmids were mixed with 1.0 μL of PLUS reagent in a 50 μL total volume reaction, with the remainder of the volume being OptiMEM (ThermoFisher/Gibco 31985062). In a separate tube, 3.8 μL of LTX reagent was mixed with 46.2 μL of OptiMEM. The DNA/PLUS Reagent mix was added to the LTX mix, pipetted up and down four times, and then incubated at room temperature for 5 min. 100 μL of this transfection mix was added drop-wise to each well of cells, which was mixed by gentle swirling. Cells were incubated in normoxia overnight and then cultured for 1-2 d in normoxia or hypoxia prior to microscopy. While the 12-well plate format was used for the oxidation/microscopy experiment, a 24-well plate format was used for the oxidation/harvest method experiment with half of the number of cells, volumes of media and reagents, and masses of DNA as listed above.

### Flow Cytometry

Cells were harvested for flow cytometry using FACS buffer (PBS pH 7.4, 2-5 mM EDTA, 0.1% BSA) or using 0.05% Trypsin-EDTA (with or without Phenol Red) for 5 min followed by quenching with medium (with or without Phenol Red). The resulting cell solution was added to at least 2 volumes of FACS buffer. Cells were spun at 150×g for 5 min, supernatant was decanted, and fresh FACS buffer was added.

Cells were harvested by trypsinizing, resuspended at approximately 10⁷ cells per mL in pre-sort medium (DMEM with 10% FBS, 25 mM HEPES (Sigma H3375), and 100 ug/mL gentamycin (Amresco 0304)), and held on ice until sorting was performed. Cells were sorted using one of several BD FACS Aria Special Order Research Products (Robert H. Lurie Cancer Center Flow Cytometry Core) with the optical configuration listed in Table 1. Cells were collected for each line in post-sort medium (DMEM with 20% FBS, 25 mM HEPES, and 100 μg/mL gentamycin), and cells were held on ice until they could be centrifuged at 150×g for 5 min and resuspended in DMEM. Cells were plated and expanded until used in experiments. Gentamycin was included in the culture medium for one week after sorting. For monoclonal cell sorting, cells were sorted directly into 96-well plates and maintained in post-sort medium until adherent, at which point the medium was changed.

Flow cytometry was run on a BD LSR Fortessa Special Order Research Product (Robert H. Lurie Cancer Center Flow Cytometry Core). Lasers and filter sets used for data acquisition are listed in Table 2 (for experiments involving reporter expression). Samples were analyzed using FlowJo v10 software (FlowJo, LLC). Fluorescence data were compensated for spectral bleed-through. The HEK293FT and B16F10 cell populations were identified by SSC-A vs. FSC-A gating, and singlets were identified by FSC-A vs. FSC-H gating. To distinguish transfected from non-transfected cells, a control sample of cells was generated by transfecting cells with a mass of pcDNA (empty vector) equivalent to the mass of DNA used in other samples in the experiment. For the single-cell subpopulation of the pcDNA-only sample, a gate was made to identify cells that were positive for the constitutive fluorescent protein used as a transfection control in other samples, such that the gate included no more than 1% of the non-fluorescent cells. To distinguish cells expressing cargo from a landing pad, a sample of the corresponding parental, non-landing pad line was used.

TABLE 1
Filters for flow cytometry sorting

| Instrument | Fluorescent protein | Parameter | Excitation laser | Filter set |
|---|---|---|---|---|
| BD FACS Aria | EBFP2, mTagBFP2 | Pacific Blue | Violet | 450/50 |
| | | | 407 nm | |
| | EYFP | FITC | Blue | 505LP, 525/30 |
| | | | 488 nm | |

TABLE 2
Filters for flow cytometry analysis

| | | Parameter/ | | |
|---|---|---|---|---|
| Instrument | Fluorescent protein | Channel name | Excitation laser | Filter set |
| BD LSR Fortessa | EBFP2, mTagBFP2 | Pacific Blue | Violet, 405 nm | 450/50 |
| | EYFP, mNeonGreen | FITC | Blue, 488 nm | 505LP, 530/30 |
| | DsRed-Express2, | PE-Texas Red | Light Green, 552 nm | 600LP, 610/20 |
| | LumiScarlet | | | |

### Flow Cytometry Data Analysis

Flow cytometry data were analyzed using FlowJo software (FlowJo, LLC) to gate on single-cell (FSC-A vs. FSC-H) and live (FSC-A vs. SSC-A) bases, compensated using compensation control samples, and gated as transfection-positive. The mean reporter signal in MFI was obtained for each sample. UltraRainbow Calibration Particles (Spherotech #URCP-100-2H) were run in each flow cytometry experiment. Beads were gated on an FSC-A vs. FSC-H basis, the nine bead subpopulations of varying intensities were identified, and the mean MFI for each subpopulation in the FITC channel and PE-Texas Red channel was obtained. These values in combination with manufacturer-supplied MEFL and MEPTR values for each subpopulation were used to fit a regression line with y-intercept equal to zero. The mean and S.E.M. for the three biological replicates were calculated. Autofluorescence background signal was subtracted using samples transfected with the transfection control marker, and error was propagated. MFI values were converted to MEFL or MEPTR using the slope of the regression line, and error was propagated.

### HEK293FT Landing Pad Integration and Selection

From exponentially growing HEK293LP cells, 0.5×105 cells were plated per well (0.5 mL medium) in 24-well format, and cells were cultured for 24 h to allow cells to attach and spread. When cells reached 50-75% confluence, Bxb1 recombinase was co-transfected with the integration vector by lipofection with Lipofectamine LTX with PLUS Reagent (ThermoFisher 15338100). 300 ng of BxB1 expression vector was mixed with 300 ng of integration vector and 0.5 μL of PLUS reagent in a 25 μL total volume reaction, with the remainder of the volume being OptiMEM (ThermoFisher/Gibco 31985062). In a separate tube, 1.9 μL of LTX reagent was mixed with 23.1 μL of OptiMEM. The DNA/PLUS Reagent mix was added to the LTX mix, pipetted up and down four times, and then incubated at room temperature for 5 min. 50 μL of this transfection mix was added drop-wise to each well of cells, which was mixed by gentle swirling. Cells were cultured until the well was ready to split (typically 3 d), without any media changes.

Cells were harvested from the 24-well plate when confluent by trypsinizing and transferring to a single well of a 6-well plate in 2 mL of medium, and then cells were cultured until they reached 50-70% confluence. Then, medium was aspirated and replaced with 2 mL of fresh media containing appropriate selection antibiotic 1 μg/mL puromycin (Invivogen ant-pr) and/or 6 μg/mL blasticidin (Alfa Aesar/ThermoFisher J61883). Medium was replaced daily with fresh medium containing antibiotics until cell death was no longer evident. Selection was first performed in puromycin for 7 d, then cells were expanded for 7 d without antibiotics. Cells were then cultured in both puromycin and blasticidin to maintain selective pressure until flow sorting. Cells were sorted as described for each line and in Flow sorting.

### B16F10 Landing Pad Integration and Selection

From exponentially growing HEK293LP cells, 0.8×105 cells were plated per well (0.5 mL medium) in 12-well format, and cells were cultured for 24 h to allow cells to attach and spread. Bxb1 recombinase was co-transfected with the integration vector by lipofection with Lipofectamine LTX with PLUS Reagent (ThermoFisher 15338100). 200 ng of BxB1 expression vector was mixed with 200 ng of integration vector and without PLUS reagent in a 50 μL total volume reaction, with the remainder of the volume being OptiMEM (ThermoFisher/Gibco 31985062). In a separate tube, 5 μL of LTX reagent was mixed with 45 μL of OptiMEM. The DNA/PLUS Reagent mix was added to the LTX mix, pipetted up and down four times, and then incubated at room temperature for 5 min. 100 μL of this transfection mix was added drop-wise to each well of cells, which was mixed by gentle swirling. Cells were cultured until the well was ready to split (typically 3 d), without any media changes.

Cells were harvested from the 12-well plate when confluent by trypsinizing and transferring to a single well of a 6-well plate in 2 mL of medium with appropriate selection antibiotic, and then cells were cultured until they reached 50-70% confluence, with frequent trypsinization to remove dead cells. Antibiotic concentrations were 1 μg/mL puromycin (Invivogen ant-pr) and 15 μg/mL blasticidin (Alfa Aesar/ThermoFisher J61883). Medium was replaced daily with fresh medium containing antibiotics until cell death was no longer evident. Cells were then cultured in both puromycin and blasticidin to maintain selective pressure until assay or flow sorting. Cells were sorted as described for each line generation and in Flow cytometry-based cell sorting.

### B16F10 Landing Pad Line Generation

From exponentially growing B16F10LP cells, 4×105 cells were plated per well (2 mL medium) in 6-well format, and cells were cultured for 24 h to allow cells to attach and spread. Cells were transfected with 160 ng each of pPD720, pPD783, and pPD864, 1520 ng of pPD005, in 100 μL total volume (balance OptiMEM), mixed with 100 μL of OptiMEM containing 10 μL of Lipofectamine LTX (total volume of 200 μL transfection mixture per well. Beginning 3 d after transfection, cells were selected with 1000 μg/mL Hygromycin (20 μL stock/mL of medium). EYFP+, single cells were sorted 18 d after transfection into a 96-well plate. Wells were visually verified to contain only 1 cell per well. Cell lines were expanded for 2-4 weeks under continuous antibiotic selection. Approximately 60 monoclonal lines were generated.

### B16F10 Landing Pad Line Validation

Validation was performed by genomic PCR through the right Rosa26 homology arm. Lines were also assessed for their ability to maintain EYFP fluorescence for 6 weeks without antibiotic selection pressure and for the performance of an integrated circuit.

### Diagrams

Genetic programs for digital functions are depicted using genetic diagrams and electronic diagrams. The former represents each promoter, protein, and regulatory interaction, and the latter represents the logic underlying these interactions.

### HEK293FT Stable Transposon Integration Cell Line Generation

From exponentially growing HEK293FT cells, 1×105 cells were plated per well (0.5 mL medium) in a 24-well format, and cells were cultured 24 h to allow cells to attach and spread. When cells reached 50-75% confluence, PiggyBac transposase was co-transfected with the integration vector by lipofection with Lipofectamine LTX with PLUS Reagent (ThermoFisher 15338100). 500 ng of PiggyBac transposon expression vector DNA was mixed with 100 ng of PiggyBac transposase DNA and 0.5 μL of PLUS reagent in a 25 μL total volume reaction, with the remaining volume as OptiME (ThermoFisher/Gibco 31985062). In a separate tube, 1.9 μL of LTX reagent was mixed with 23.1 μL of OptiMEM. The DNA/PLUS Reagent mix was added to the LTX mix. pipetted up and down four times, and then incubated at room temperature for 5 min. 50 μL of this transfection mix was added drop-wise to each well of cells, which was mixed by gentle swirling. Cells were cultured until the well was ready to split (typically 3 d), without any media changes.

Cells were harvested from the 24-well plate when confluent by trypsinizing and transferring to a single well of a 12-well plate in 1 mL of medium, and then cells were cultured until they reached 50-70% confluence. Then, medium was aspirated and replaced with 1 mL of fresh media containing appropriate selection antibiotic 1 μg/mL puromycin (Invivogen ant-pr). Medium was replaced daily with fresh medium containing antibiotics until cell death was no longer evident. Selection was performed in puromycin for 14 d, and cells were expanded under antibiotic selection for another 7 d.

### HEK293FT Hypoxia Time Course Experiment

For the 4 d time course assay, stable cell lines were plated in 0.5 mL of DMEM in triplicate in 24-well format with 2×10⁴ cells/well. Cells cultured in normoxia 24 h to allow cells to adhere and spread. Cells were subsequently cultured under normoxic or hypoxic conditions for 4 d, and each day (24 h), cells were harvested and stained for CAR using biotinylated anti-human EGF antibody (R&D Systems BAF236) and streptavidin-APC (Abcam ab243099).

### Example 2—Hypoxia Biosensor Performance is not Enhanced by Increasing the Number of Biosensors in the Cell

The present example illustrates that hypoxia biosensor performance is not enhanced (i.e., made more sensitive to hypoxia detection or more specific to hypoxia or the reporter gene output) simply by increasing the copy number of the hypoxia biosensor in a cell. Each cell line tested expressed a hypoxia biosensor with one of three minimal promoters (SV40, CMV, or YB_TATA).

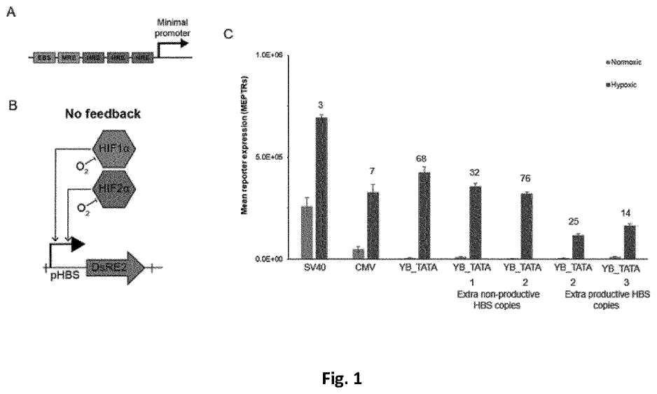

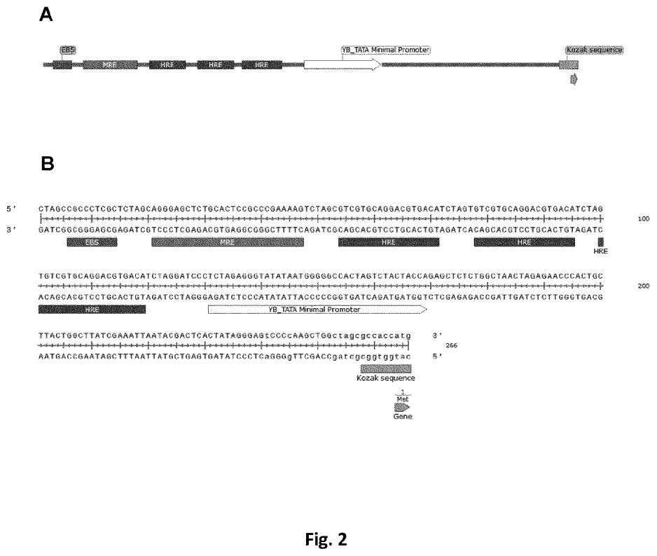

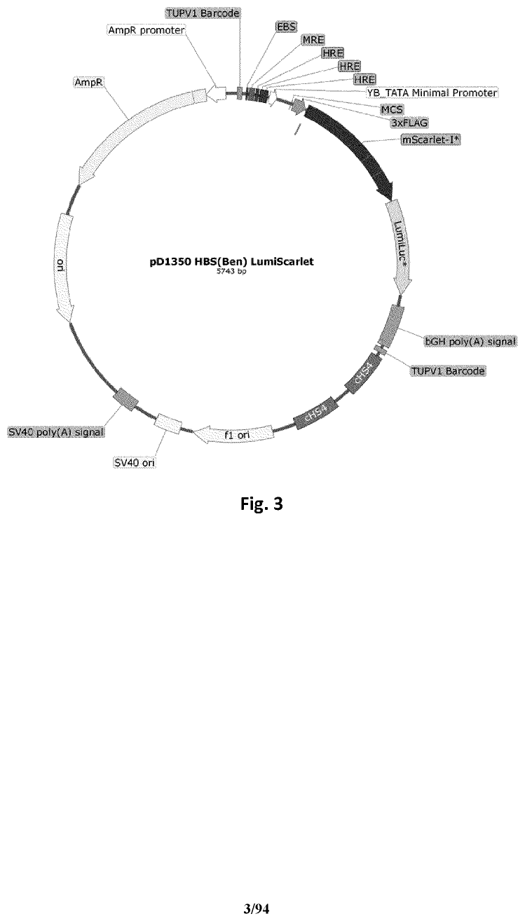

FIG. 1A shows a schematic illustrating the composition of the hypoxia biosensor DNA sequence comprising an EBS, an MRE, and three HREs 5′ to a minimal promoter sequence. The structure of the hypoxia biosensors are illustrated in greater detail in FIG. 2. Hypoxia biosensors can be provided to a cell on a plasmid. FIG. 3 shows an annotated map of a plasmid contain a HBS containing a YB_TATA minimal promoter. FIG. 1B illustrates the hypoxia biosensor with a downstream DsRed-Express2 reporter protein. The hypoxia biosensor is activated by HIF1α and HIF2α, which are degraded in the presence of oxygen. FIG. 1C shows the results of an experiment in which B16F10 cells with hypoxia biosensors integrated into the genome were cultured under normoxic or hypoxic conditions, and reporter expression was analyzed by flow cytometry. Fold induction is shown over the hypoxic bar. Each cell line tested expressed a hypoxia biosensor with one of three minimal promoters (SV40, CMV, or YB_TATA).

For cell lines containing the YB_TATA-based hypoxia biosensor, additional cell lines were tested that each contained either 1 or 2 additional copies of the hypoxia biosensor with (“productive”) or without (“non-productive) a downstream reporter. While the choice of minimal promoter impacted the levels of gene expression, adding additional productive HBS copies did not increase gene expression. Adding additional productive or non-productive copies actually led to decreased levels of reporter expression. In the case of the addition of productive copies, this was unexpected.

### Example 3—Mutant Stable HIF1α Leads to Higher Levels of Gene Expression from the Hypoxia Biosensor than does Hypoxic Culture

The present example shows the surprising and unexpected result that oxygen-insensitive HIF1α drives more robust report expression than hypoxic culture conditions alone and conditions in which the cells are cultured with the hypoxia mimetic cobalt.

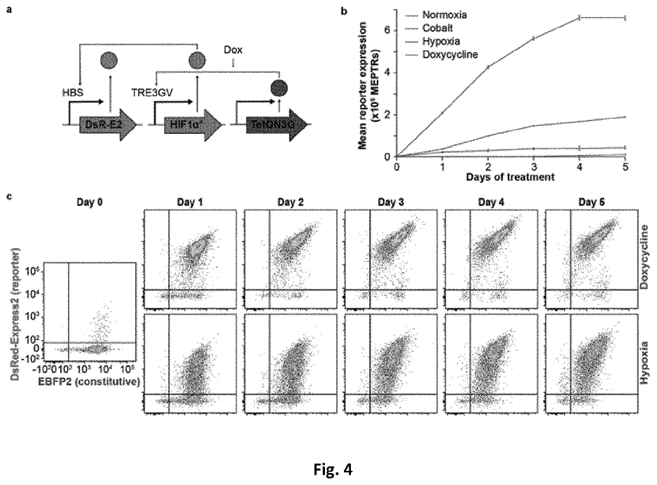

In these experiments, while the YB_TATA-based construct led to a high fold induction, the level of induced signaling was relatively low compared to the CMV_min construct, indicating that it may be possible to increase the level of hypoxia-signaling from the YB_TATA-based construct. In this experiment, a mutant, oxygen-insensitive version of HIF1α (HIF1α*) that has previously been shown to induce gene expression in the presence of oxygen, as the proline residues that are normally oxidized have been mutated to alanine residues (See Hu et al., Mol Biol Cell 18, 4528-4542 (2007)), was included as a positive control. The mutant HIF1α gene was placed under the control of a doxycycline-responsive promoter and integrated a construct including this transcription unit, the HBS, and a constitutive EBFP2 gene into HEK293FT-LP cells (FIG. 4A). Following selection, flow sorting, and recovery, the cell line was cultured with cobalt (a hypoxia mimetic), hypoxia, or doxycycline. Culture with doxycycline, leading to expression of the oxygen-insensitive HIF1α*, surprisingly led to higher levels of induced reporter gene expression than culture with cobalt or in the presence of hypoxia (FIG. 4B) and the resulting population was more homogenous (FIG. 4C). This indicated that in HEK293FT cells, the supply of HIF1α limits HBS performance. This result provided guidance for the design of positive feedback circuits with HIF1α and HIF2α

### Example 4—Hypoxia Biosensor Performance can be Enhanced with Positive Feedback with HIF1α and HIF2α

The present Example illustrates that HBS performance can be enhanced with the incorporation of positive feedback elements, in this case those incorporating exogenous expression of native genes.

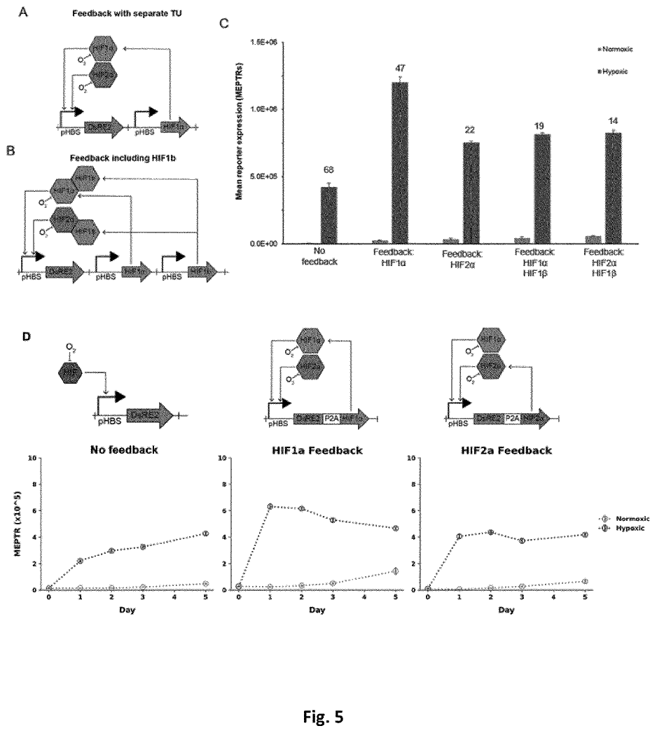

The unexpected findings presented in Example 3 suggested that removal of the above-mentioned limitation of the endogenous level of HIF1α could result in a more sensitive and responsive hypoxia biosensor. One way in which this could be accomplished would be to create positive feedback circuits that, following initial biosensor activation, promote further biosensor activation. To test this hypothesis, several feedback circuits that would produce more wild type HIF1α, HIF2α, and/or HIF1β upon activation of the biosensor were constructed, of which several examples are shown in FIG. 5. Circuits containing additional HIF1β were included in the designs, as this protein dimerizes with HIF1a and HIF2α, suggesting that the endogenous levels of HIF1β may be limiting as well. Here, the use of positive feedback is preferable to constitutive expression of these components, as this might overwhelm the cell's ability to hydroxylate and degrade these components in the presence of oxygen, leading to nonspecific HBS activation. It is also preferable to positive feedback with the stable mutant components, as leaky expression of these from the HBS could lead to a self-propagating feedback loop, also leading to nonspecific HBS activation.

FIG. 5A illustrates a schematic of a hypoxia biosensor that is enhanced by positive feedback with HIF1α. The HIF1α sequence used was that of SEQ ID NO: 1. In SEQ ID NO: 1-5, the genes are codon optimized, both in order to increase the level of gene expression and in order to avoid negative regulation of the mRNA by anti-sense or other interfering RNAs that the cell may employ to shut down the hypoxia response. FIG. 5B illustrates a schematic of a hypoxia biosensor that is enhanced by positive feedback with HIF1α and HIF1β. The HIF1α and HIF1β sequences used were those of SEQ ID NO: 1 and SEQ ID NO: 5, respectively. FIG. 5C. B16F10 cells with hypoxia biosensors with or without several feedback strategies integrated into their genomes were cultured under normoxic or hypoxic conditions and reporter expression was then analyzed by flow cytometry. Fold induction is shown over the hypoxic bar. Each cell line tested had a hypoxia biosensor with the YB_TATA minimal promoter. All feedback designs tested increased the level of hypoxia-induced gene expression from the hypoxia biosensor. Unexpectedly, the inclusion of positive feedback with HIF1β in addition to HIF1α or HIF2α did not lead to further increases in reporter output compared to conditions with positive feedback circuits containing only HIF1α or HIF2α. FIG. 5D shows the results of experiments in which B16F10 cells with hypoxia biosensors with or without several feedback strategies integrated into their genomes were cultured under normoxic or hypoxic conditions, and reporter expression was analyzed by flow cytometry daily for up to 5 days. The HIF1α and HIF2α sequences used were those of SEQ ID NO: 1 and SEQ ID NO: 3, respectively. Each cell line tested had a hypoxia biosensor with the YB_TATA minimal promoter. Schematics illustrating each biosensor design are shown above the corresponding data. All feedback designs tested increased the level of hypoxia-induced gene expression from the hypoxia biosensor and the speed with which maximal gene expression from the biosensor was reached.

SEQ ID NO: 1
Nucleotide sequence of codon-optimized murine HIF1α
ATGGAAGGTGCTGGCGGCGAGAACGAGAAGAAAAAGATGTCCAGCGAGCGCCGGAAAGAGAAGTCTAGGGACGCCGC
CAGAAGCAGAAGATCCAAAGAAAGCGAGGTGTTCTACGAGCTGGCCCACCAACTGCCTCTGCCTCACAATGTGTCTA
GCCACCTGGACAAGGCCAGCGTGATGAGACTGACCATCAGCTACCTGAGAGTGCGGAAGCTGCTGGATGCTGGCGGA
CTGGATAGCGAGGATGAGATGAAGGCCCAGATGGACTGCTTCTACCTGAAGGCCCTGGACGGCTTCGTGATGGTGCT
GACAGACGATGGCGACATGGTGTATATCAGCGACAACGTGAACAAGTACATGGGGCTGACCCAGTTCGAGCTGACAG
GCCACAGCGTGTTCGACTTCACACACCCCTGTGACCACGAAGAGATGAGAGAGATGCTGACCCACAGAAACGGCCCC
GTGCGGAAGGGAAAAGAGCTGAATACCCAGCGGTCATTCTTCCTGAGGATGAAGTGTACCCTGACCAGCAGGGGCAG
AACCATGAACATCAAGAGCGCTACCTGGAAAGTGCTGCACTGCACCGGACACATCCACGTGTACGACACCAACAGCA
ACCAGCCTCAGTGCGGCTACAAGAAACCTCCTATGACCTGCCTGGTGCTGATCTGCGAGCCCATTCCTCATCCTAGC
AACATCGAGATCCCTCTGGACAGCAAGACCTTCCTGAGCAGACACAGCCTGGACATGAAGTTCAGCTACTGCGACGA
GAGAATCACCGAGCTGATGGGCTACGAGCCCGAGGAACTTCTCGGCAGATCCATCTATGAGTATTACCACGCTCTGG
ACTCCGACCACCTGACCAAGACACACCACGATATGTTCACCAAGGGCCAAGTGACCACCGGCCAGTACAGAATGCTG
GCTAAGAGAGGCGGCTACGTCTGGGTCGAGACACAGGCTACCGTGATCTACAACACCAAGAACTCCCAGCCACAGTG
CATCGTGTGCGTGAACTACGTCGTGTCCGGGATCATCCAGCACGACCTGATCTTTAGCCTGCAACAGACCGAGAGCG
TGCTGAAGCCTGTGGAAAGCAGCGACATGAAGATGACCCAGCTGTTCACAAAGGTGGAATCCGAGGACACCAGCTGC
CTGTTCGACAAGCTGAAGAAAGAGCCCGACGCTCTGACACTGCTGGCTCCAGCTGCTGGCGACACAATCATCAGCCT
GGATTTCGGCAGCGACGACACCGAGACAGAGGATCAGCAGCTCGAAGATGTGCCCCTGTATAACGACGTGATGTTCC
CCAGCAGCAACGAGAAGCTGAATATCAACCTGGCCATGTCTCCTCTGCCTAGCTCCGAGACACCCAAGCCTCTGAGA
AGCTCTGCCGATCCTGCTCTGAATCAAGAGGTGGCCCTGAAGCTGGAAAGCTCCCCTGAATCTCTGGGCCTGAGCTT
CACCATGCCTCAGATCCAGGACCAGCCTGCCTCTCCTTCTGACGGCTCTACAAGACAGAGCAGCCCCGAGAGACTGC
TGCAAGAGAATGTGAACACCCCAAACTTCTCCCAGCCTAACAGCCCCAGCGAGTATTGCTTCGACGTGGACAGCGAT
ATGGTCAACGTGTTCAAGCTGGAACTGGTGGAAAAGCTGTTCGCCGAGGATACCGAGGCTAAGAACCCCTTCAGCAC
CCAAGACACCGACCTGGACCTTGAGATGCTGGCCCCTTACATCCCTATGGACGACGACTTCCAGCTGAGATCCTTCG
ACCAGCTGAGCCCACTGGAAAGCAACAGCCCATCTCCACCTAGCATGAGCACCGTGACCGGCTTTCAGCAGACTCAG
CTCCAGAAGCCTACCATCACCGCCACCGCCACAACAACAGCCACCACAGACGAGAGCAAGACCGAGACTAAGGACAA
CAAAGAGGACATCAAGATCCTGATCGCCTCTCCAAGCAGCACCCAGGTGCCACAAGAGACAACCACAGCCAAGGCCT
CTGCTTACAGCGGCACCCACAGCAGAACCGCTTCTCCAGATAGAGCCGGCAAGAGAGTGATCGAGCAGACCGACAAG
GCTCACCCTAGAAGCCTGAACCTGAGCGCCACACTGAACCAGAGAAACACCGTGCCTGAGGAAGAACTGAACCCCAA
GACAATCGCCAGCCAGAACGCCCAGAGAAAGCGGAAGATGGAACACGACGGCAGCCTGTTCCAGGCTGCTGGAATCG
GAACACTGCTTCAGCAGCCAGGCGACTGTGCCCCTACAATGAGCCTGAGCTGGAAGCGCGTGAAGGGCTTCATCAGC
TCTGAGCAGAACGGCACCGAGCAGAAAACCATCATTCTGATCCCCAGCGACCTGGCCTGTAGACTGCTGGGCCAATC
TATGGATGAGAGCGGCCTGCCTCAGCTGACCAGCTACGACTGTGAAGTGAACGCCCCTATCCAGGGCAGCAGAAACC
TGCTCCAGGGCGAAGAACTGCTGAGAGCCCTGGACCAAGTGAACTGA
SEQ ID NO: 2
Amino acid sequence of codon-optimized murine HIF1α
MEGAGGENEKKKMSSERRKEKSRDAARSRRSKESEVFYELAHQLPLPHNVSSHLDKASVMRLTISYLRVRKLLDAGG
LDSEDEMKAQMDCFYLKALDGFVMVLTDDGDMVYISDNVNKYMGLTQFELTGHSVFDFTHPCDHEEMREMLTHRNGP
VRKGKELNTQRSFFLRMKCTLTSRGRTMNIKSATWKVLHCTGHIHVYDTNSNQPQCGYKKPPMTCLVLICEPIPHPS
NIEIPLDSKTFLSRHSLDMKFSYCDERITELMGYEPEELLGRSIYEYYHALDSDHLTKTHHDMFTKGQVTTGQYRML
AKRGGYVWVETQATVIYNTKNSQPQCIVCVNYVVSGIIQHDLIFSLQQTESVLKPVESSDMKMTQLFTKVESEDTSC
LFDKLKKEPDALTLLAPAAGDTIISLDFGSDDTETEDQQLEDVPLYNDVMFPSSNEKLNINLAMSPLPSSETPKPLR
SSADPALNQEVALKLESSPESLGLSFTMPQIQDQPASPSDGSTRQSSPERLLQENVNTPNFSQPNSPSEYCFDVDSD
MVNVFKLELVEKLFAEDTEAKNPFSTQDTDLDLEMLAPYIPMDDDFQLRSFDQLSPLESNSPSPPSMSTVTGFQQTQ
LQKPTITATATTTATTDESKTETKDNKEDIKILIASPSSTQVPQETTTAKASAYSGTHSRTASPDRAGKRVIEQTDK
AHPRSLNLSATLNQRNTVPEEELNPKTIASQNAQRKRKMEHDGSLFQAAGIGTLLQQPGDCAPTMSLSWKRVKGFIS
SEQNGTEQKTIILIPSDLACRLLGQSMDESGLPQLTSYDCEVNAPIQGSRNLLQGEELLRALDQVN*
SEQ ID NO: 3
Nucleotide sequence of codon-optimized murine HIF2α
ATGACAGCCGACAAAGAGAAGAAGAGAAGCTCCAGCGAGCTGCGGAAAGAGAAGTCTAGGGACGCCGCCAGATGCAG
AAGATCCAAAGAAACCGAGGTGTTCTACGAGCTGGCCCACGAACTGCCTCTGCCTCACTCTGTGTCTAGCCACCTGG
ACAAGGCCAGCATCATGAGACTGGCTATCAGCTTCCTGAGAACCCACAAGCTGCTGTCCAGCGTGTGCAGCGAGAAC
GAGTCTGAGGCCGAAGCCGACCAGCAGATGGACAACCTGTACCTGAAGGCCCTGGAAGGCTTTATCGCCGTGGTCAC
ACAGGACGGCGATATGATCTTCCTGAGCGAGAACATCAGCAAGTTCATGGGCCTGACACAGGTGGAACTGACCGGCC
ACAGCATCTTCGACTTCACACACCCCTGCGACCACGAGGAAATCAGAGAGAACCTGACACTGAAGAACGGCTCTGGC
TTCGGCAAGAAGTCCAAGGATGTGTCTACCGAGAGGGACTTCTTCATGCGGATGAAGTGTACCGTGACCAACAGAGG
CAGAACCGTGAACCTGAAGTCCGCCACATGGAAGGTGCTGCACTGTACCGGACAAGTGCGGGTCTACAACAACTGCC
CTCCTCACAGCAGCCTGTGCGGCTCTAAAGAGCCTCTGCTGAGCTGCCTGATCATCATGTGCGAGCCCATCCAGCAT
CCTTCTCACATGGACATCCCTCTGGACTCCAAGACCTTCCTGTCCAGACACAGCATGGACATGAAGTTCACCTACTG
CGACGACCGCATCCTGGAACTGATCGGATATCACCCCGAGGAACTGCTGGGCAGAAGCGCCTACGAGTTCTACCACG
CTCTGGATTCCGAGAACATGACCAAGAGCCACCAGAACCTGTGTACCAAAGGCCAGGTGGTGTCCGGCCAGTACAGA
ATGCTGGCTAAGCACGGCGGCTACGTGTGGCTGGAAACACAGGGCACCGTGATCTACAACCCCAGAAACCTCCAGCC
TCAGTGCATTATGTGCGTGAACTACGTGCTGTCCGAGATCGAGAAGAACGACGTGGTGTTCAGCATGGATCAGACCG
AGAGCCTGTTCAAGCCCCACCTGATGGCCATGAACTCTATCTTCGACAGCAGCGACGACGTGGCCGTGACCGAAAAG
AGCAACTACCTGTTCACCAAGCTGAAAGAGGAACCTGAGGAACTGGCCCAGCTGGCTCCTACACCTGGCGACGCTAT
CATCAGCCTGGACTTCGGCAGCCAGAACTTCGACGAGCCTTCTGCCTACGGCAAGGCCATCCTTCCACCTGGGCAAC
CTTGGGTTTCAGGACTGAGAAGCCACAGCGCCCAGAGCGAGTCTGGATCTCTGCCTGCTTTCACCGTGCCTCAGGCC
GACACACCAGGCAACACAACACCTAGCGCCAGCAGCAGCTCCAGCTGTTCTACACCTAGCAGCCCCGAGGACTACTA
CAGCTCCCTGGAAAACCCTCTGAAGATCGAAGTCATCGAGAAGCTGTTCGCTATGGACACCGAGCCTAGAGATCCTG
GCAGCACCCAGACAGACTTCTCCGAGCTGGACCTGGAAACCCTGGCTCCTTACATCCCAATGGACGGCGAGGACTTC
CAGCTGAGCCCTATCTGTCCAGAGGAACCCCTGATGCCTGAGAGCCCTCAGCCTACACCTCAGCACTGCTTCAGCAC
CATGACCTCCATCTTCCAGCCTCTGACACCCGGCGCTACACACGGCCCATTCTTTCTGGACAAGTACCCTCAGCAGC
TGGAAAGCAGAAAGACCGAGTCCGAGCACTGGCCTATGTCTAGCATCTTCTTCGACGCCGGCAGCAAGGGCAGCCTG
TCTCCTTGTTGTGGCCAGGCCTCTACACCCCTGTCCTCTATGGGCGGCAGATCCAACACACAGTGGCCTCCAGATCC
TCCTCTGCACTTCGGCCCTACAAAATGGCCTGTGGGCGATCAGTCTGCCGAAAGCCTTGGAGCACTGCCTGTGGGAT
CTTCCCAGCTGGAACCTCCTTCTGCTCCTCCACACGTGTCCATGTTCAAGATGAGAAGCGCCAAGGACTTTGGCGCT
AGAGGCCCCTACATGATGAGCCCTGCTATGATCGCCCTGAGCAACAAGCTGAAGCTGAAGAGACAGCTGGAATACGA
GGAACAGGCTTTCCAGGACACCAGCGGCGGAGATCCACCTGGCACATCTAGCTCTCACCTGATGTGGAAGAGGATGA
AGTCCCTGATGGGCGGAACATGCCCTCTGATGCCCGACAAGACAATCAGCGCCAACATGGCCCCTGACGAGTTCACC
CAGAAATCCATGAGAGGACTGGGCCAGCCACTGAGACATCTGCCTCCACCTCAACCTCCAAGCACCAGAAGCTCTGG
CGAGAACGCCAAGACAGGCTTCCCACCTCAGTGTTACGCCAGTCAGTTTCAGGATTACGGCCCTCCTGGCGCTCAGA
AAGTGTCTGGCGTTGCAAGCAGACTGCTGGGACCTAGCTTCGAGCCTTACCTGCTGCCTGAGCTGACCAGATACGAC
TGCGAAGTGAACGTGCCAGTGCCTGGCAGCTCTACACTTCTGCAAGGCAGGGATCTGCTGCGCGCACTGGATCAGGC
TACATGA
SEQ ID NO: 4
Amino acid sequence of codon-optimized murine HIF2α
MTADKEKKRSSSELRKEKSRDAARCRRSKETEVFYELAHELPLPHSVSSHLDKASIMRLAISFLRTHKLLSSVCSEN
ESEAEADQQMDNLYLKALEGFIAVVTQDGDMIFLSENISKFMGLTQVELTGHSIFDFTHPCDHEEIRENLTLKNGSG
FGKKSKDVSTERDFFMRMKCTVTNRGRTVNLKSATWKVLHCTGQVRVYNNCPPHSSLCGSKEPLLSCLIIMCEPIQH
PSHMDIPLDSKTFLSRHSMDMKFTYCDDRILELIGYHPEELLGRSAYEFYHALDSENMTKSHQNLCTKGQVVSGQYR
MLAKHGGYVWLETQGTVIYNPRNLQPQCIMCVNYVLSEIEKNDVVFSMDQTESLFKPHLMAMNSIFDSSDDVAVTEK
SNYLFTKLKEEPEELAQLAPTPGDAIISLDFGSQNFDEPSAYGKAILPPGQPWVSGLRSHSAQSESGSLPAFTVPQA
DTPGNTTPSASSSSSCSTPSSPEDYYSSLENPLKIEVIEKLFAMDTEPRDPGSTQTDFSELDLETLAPYIPMDGEDF
QLSPICPEEPLMPESPQPTPQHCFSTMTSIFQPLTPGATHGPFFLDKYPQQLESRKTESEHWPMSSIFFDAGSKGSL
SPCCGQASTPLSSMGGRSNTQWPPDPPLHFGPTKWPVGDQSAESLGALPVGSSQLEPPSAPPHVSMFKMRSAKDFGA
RGPYMMSPAMIALSNKLKLKRQLEYEEQAFQDTSGGDPPGTSSSHLMWKRMKSLMGGTCPLMPDKTISANMAPDEFT
QKSMRGLGQPLRHLPPPQPPSTRSSGENAKTGFPPQCYASQFQDYGPPGAQKVSGVASRLLGPSFEPYLLPELTRYD
CEVNVPVPGSSTLLQGRDLLRALDQAT*
SEQ ID NO: 5
Nucleotide sequence of codon-optimized murine HIF1β
ATGGCCGCCACCACCGCCAATCCTGAGATGACCTCTGATGTGCCCAGCCTGGGACCTACAATCGCCTCTGGAAATCC
CGGACCTGGCATTCAAGGTGGCGGAGCTGTTGTGCAGAGAGCCATCAAGAGAAGAAGCGGCCTGGACTTCGACGACG
AGGTGGAAGTGAACACCAAGTTTCTGAGATGCGACGACGACCAGATGTGCAACGACAAAGAGAGATTCGCCAGATCC
GACGATGAGCAGAGCAGCGCCGACAAAGAAAGGCTGGCCAGAGAGAACCACAGCGAGATCGAGAGAAGGCGCAGAAA
CAAGATGACCGCCTACATCACCGAGCTGAGCGACATGGTGCCTACCTGTTCTGCCCTGGCCAGAAAGCCTGACAAGC
TGACCATCCTGAGAATGGCCGTGTCTCACATGAAGTCCCTGAGAGGCACCGGCAACACAAGCACAGACGGCAGCTAC
AAGCCCAGCTTCCTGACCGACCAAGAGCTGAAGCACCTGATCCTGGAAGCCGCCGATGGCTTCCTGTTCATCGTGTC
TTGCGAGACAGGCAGAGTGGTGTATGTGTCCGACAGCGTGACCCCTGTGCTGAACCAGCCTCAGTCTGAGTGGTTCG
GCAGCACCCTGTACGACCAGGTTCACCCTGACGATGTGGATAAGCTGAGAGAGCAGCTCAGCACCAGCGAGAACGCT
CTGACTGGCAGAGTGCTGGACCTGAAAACCGGCACCGTGAAGAAAGAGGGCCAGCAGTCCAGCATGAGAATGTGCAT
GGGCAGCCGTAGATCCTTCATCTGCCGGATGAGATGCGGCACCTCTAGCGTGGACCCTGTGTCTATGAACAGACTGT
CCTTCCTGAGAAACAGATGCAGAAACGGCCTGGGCAGCGTGAAAGAAGGCGAGCCTCACTTCGTGGTGGTGCATTGC
ACCGGCTACATCAAGGCTTGGCCTCCAGCCGGTGTCAGCCTGCCTGATGATGATCCTGAAGCTGGACAGGGCAGCAA
GTTCTGCCTGGTGGCTATCGGCAGACTGCAAGTGACAAGCAGCCCCAACTGCACCGACATGAGCAACATCTGCCAGC
CTACCGAGTTCATCAGCAGACACAACATCGAGGGCATCTTCACCTTCGTGGACCACAGATGTGTGGCCACCGTGGGC
TATCAGCCTCAAGAGCTGCTGGGCAAGAACATCGTCGAGTTCTGTCACCCCGAGGATCAGCAGCTTCTGAGGGACAG
CTTTCAGCAGGTCGTGAAGCTGAAAGGCCAGGTGCTGTCCGTGATGTTCAGATTCAGATCCAAGACCAGAGAATGGC
TGTGGATGAGAACAAGCAGCTTCACATTTCAGAACCCCTACTCCGATGAGATCGAGTACATCATCTGCACCAACACC
AACGTGAAGAACAGCAGCCAAGAGCCTAGACCTACACTGAGCAACACAATCCCCAGAAGCCAGCTCGGCCCTACAGC
CAACCTGAGTCTGGAAATGGGCACCGGACAGCTCCCTAGCAGACAGCAGCAGCAACAGCACACCGAACTGGATATGG
TGCCCGGCAGAGATGGCCTGGCCAGCTACAATCACTCTCAGGTGTCAGTGCAGCCTGTGGCCTCTGCTGGAAGCGAG
CATAGCAAGCCCCTGGAAAAGAGCGAGGGCCTGTTCGCCCAGGACAGGGACCCTAGATTCCCCGAAATCTACCCCAG
CATCACCGCCGACCAGAGCAAGGGCATCAGCTCTAGTACAGTGCCCGCCACACAGCAGCTTTTCAGCCAGGGCTCTA
GCTTCCCACCTAATCCTAGACCTGCCGAGAACTTCAGAAACAGCGGCCTGACACCTCCTGTGACCATCGTGCAGCCT
TCTAGCTCTGCCGGACAGATCCTGGCTCAGATCAGCAGGCACAGCAACCCTGCTCAGGGCTCTGCTCCTACCTGGAC
CTCTTCTAGCAGACCTGGCTTCGCCGCTCAGCAGGTTCCAACACAGGCTACCGCCAAGACAAGATCCAGCCAGTTCG
GCGTGAACAACTTCCAGACCAGCTCCAGCTTCTCCGCCATGTCTTTGCCTGGCGCTCCTACAGCCTCTTCTGGCACA
GCAGCATACCCCGCTCTGCCTAACAGAGGCAGCAACTTCCCACCAGAGACAGGCCAGACAACCGGCCAGTTCCAGGC
TAGAACAGCCGAAGGTGTTGGCGTGTGGCCTCAGTGGCAAGGACAGCAGCCTCACCACAGAAGCAGCTCTAGCGAGC
AGCACGTGCAGCAGACTCAAGCCCAGGCTCCATCTCAGCCCGAGGTGTTCCAAGAGATGCTGTCCATGCTGGGCGAC
CAGTCCAACACCTACAACAACGAAGAGTTCCCCGACCTGACAATGTTCCCTCCATTCAGCGAATAG
SEQ ID NO: 6
Amino acid sequence of codon-optimized murine HIF1β
MAATTANPEMTSDVPSLGPTIASGNPGPGIQGGGAVVQRAIKRRSGLDFDDEVEVNTKFLRCDDDQMCNDKERFARS
DDEQSSADKERLARENHSEIERRRRNKMTAYITELSDMVPTCSALARKPDKLTILRMAVSHMKSLRGTGNTSTDGSY
KPSFLTDQELKHLILEAADGFLFIVSCETGRVVYVSDSVTPVLNQPQSEWFGSTLYDQVHPDDVDKLREQLSTSENA
LTGRVLDLKTGTVKKEGQQSSMRMCMGSRRSFICRMRCGTSSVDPVSMNRLSFLRNRCRNGLGSVKEGEPHFVVVHC
TGYIKAWPPAGVSLPDDDPEAGQGSKFCLVAIGRLQVTSSPNCTDMSNICQPTEFISRHNIEGIFTFVDHRCVATVG
YQPQELLGKNIVEFCHPEDQQLLRDSFQQVVKLKGQVLSVMFRFRSKTREWLWMRTSSFTFQNPYSDEIEYIICTNT
NVKNSSQEPRPTLSNTIPRSQLGPTANLSLEMGTGQLPSRQQQQQHTELDMVPGRDGLASYNHSQVSVQPVASAGSE
HSKPLEKSEGLFAQDRDPRFPEIYPSITADQSKGISSSTVPATQQLFSQGSSFPPNPRPAENFRNSGLTPPVTIVQP
SSSAGQILAQISRHSNPAQGSAPTWTSSSRPGFAAQQVPTQATAKTRSSQFGVNNFQTSSSFSAMSLPGAPTASSGT
AAYPALPNRGSNFPPETGQTTGQFQARTAEGVGVWPQWQGQQPHHRSSSSEQHVQQTQAQAPSQPEVFQEMLSMLGD
QSNTYNNEEFPDLTMFPPFSE*

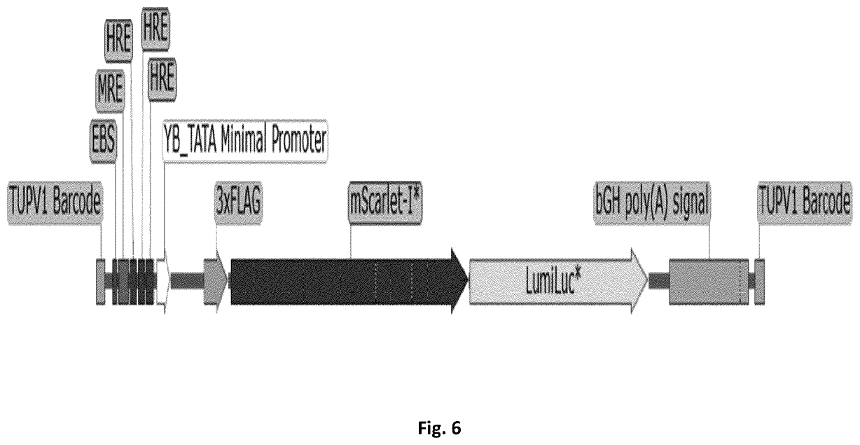

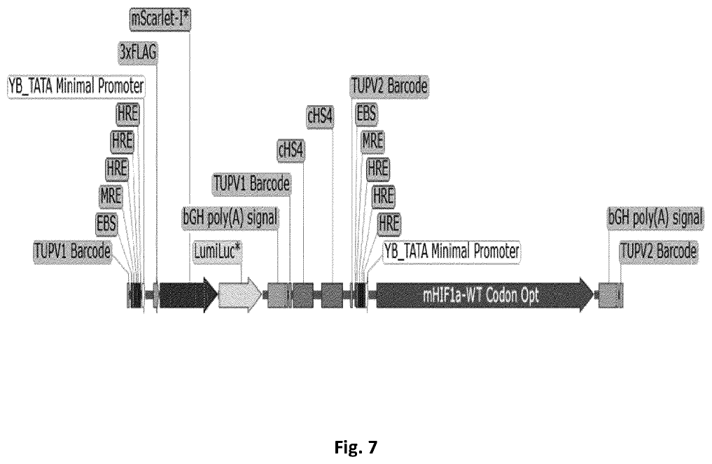

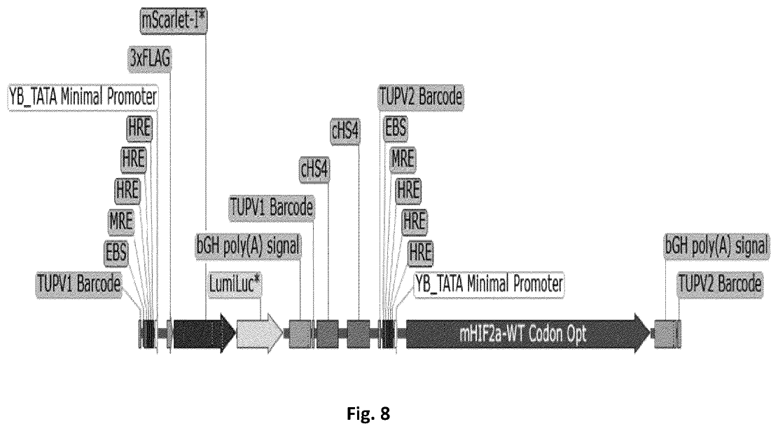

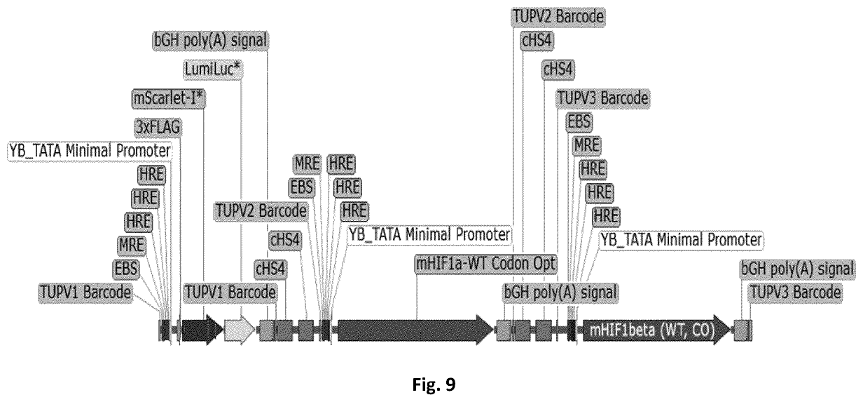

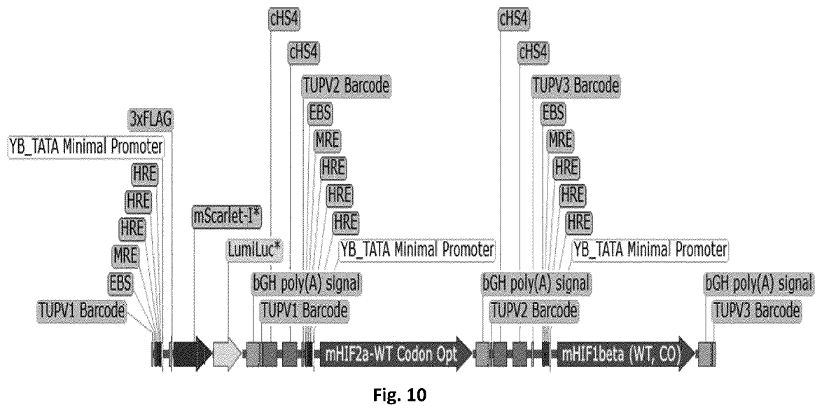

FIG. 6 shows an annotated map of a DNA sequence containing a hypoxia biosensor that does not employ any feedback elements, such as that shown in FIG. 5C, D. FIG. 7 shows an annotated map of a DNA sequence containing a hypoxia biosensor enhanced by positive feedback with HIF1α, such as that shown in FIG. 5A, C, D. FIG. 8 shows an annotated map of a DNA sequence containing a hypoxia biosensor enhanced by positive feedback with HIF2α, such as that shown in FIG. 5C, D. FIG. 9 shows an annotated map of a DNA sequence containing a hypoxia biosensor enhanced by positive feedback with HIF1α and HIF1β, such as that shown in FIG. 5B, C. FIG. 10 shows an annotated map of a DNA sequence containing a hypoxia biosensor enhanced by positive feedback with HIF2α and HIF1β, such as that shown in FIG. 5C.

### Example 5—Oxygen Degradation Tags Confer Oxygen Sensitivity to COMET TFs

Dysregulation of the HIF response is a vital step in the formation in many cancers and each is likely dysregulated differently; genetic circuits that rely on these endogenous components to provide positive feedback are therefore unlikely to be robust to the cell type in which they operate. Accordingly, the design of genetic circuits to modulate the response of the HBS that do not rely on the endogenous HIF proteins is desirable. Provided herein are designed feedback circuits that use Composable Mammalian Elements of Transcription (COMET) transcription factors (TFs), with multiple different designs imparting amplification of and/or positive feedback to hypoxia biosensors, resulting in increased hypoxia-induced signaling.

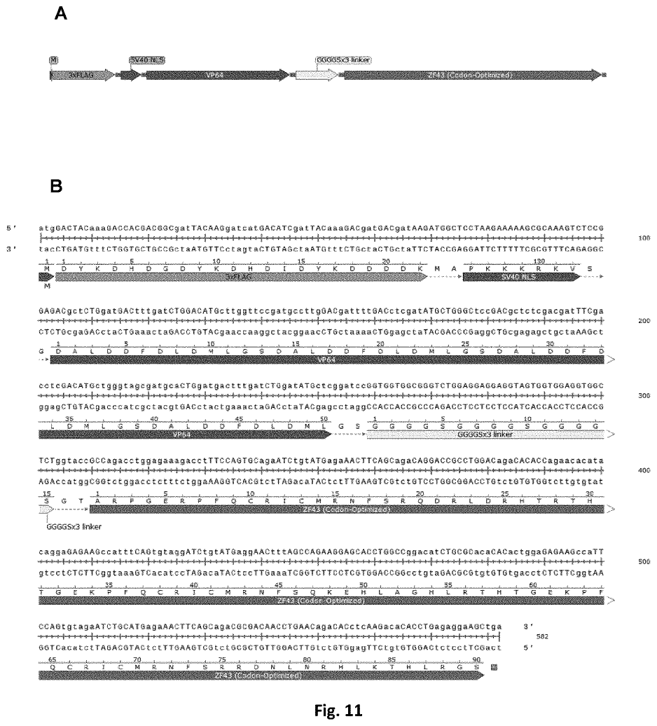

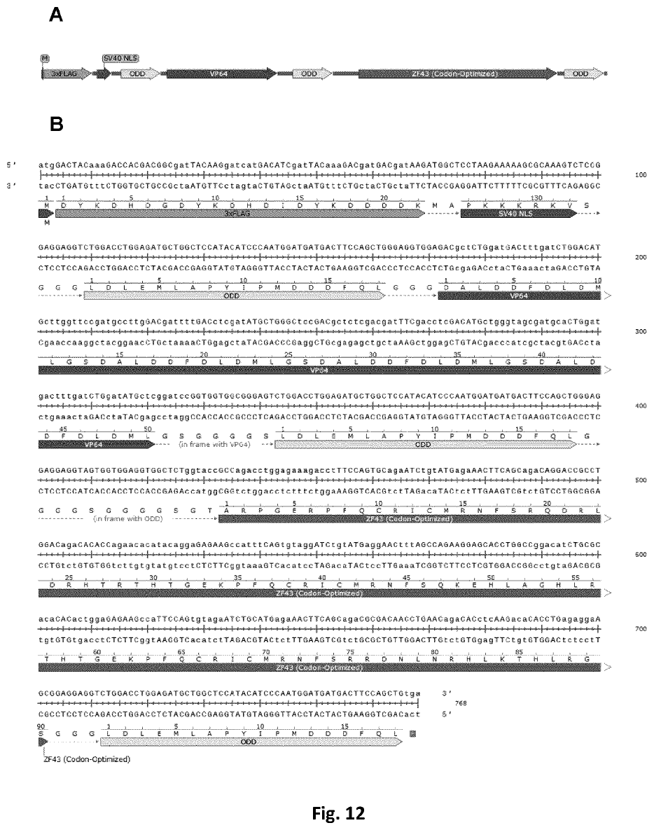

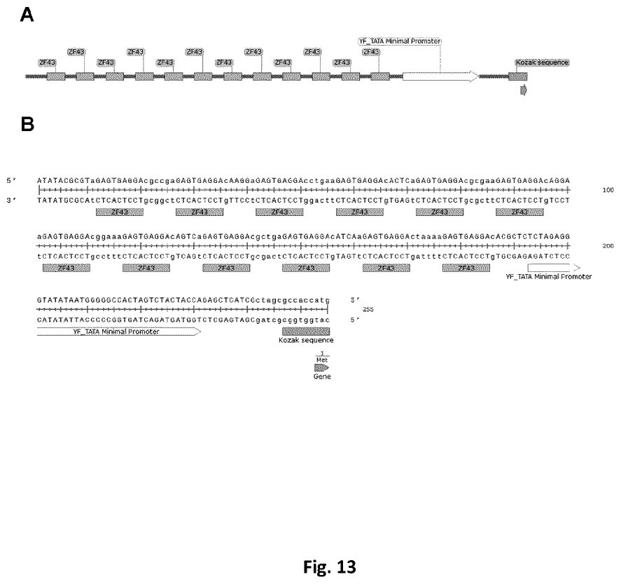

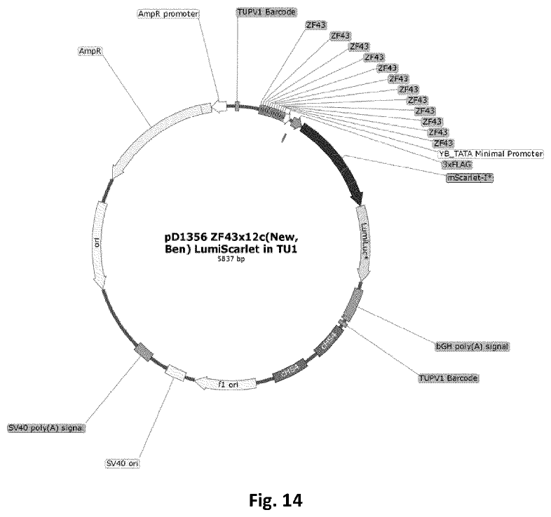

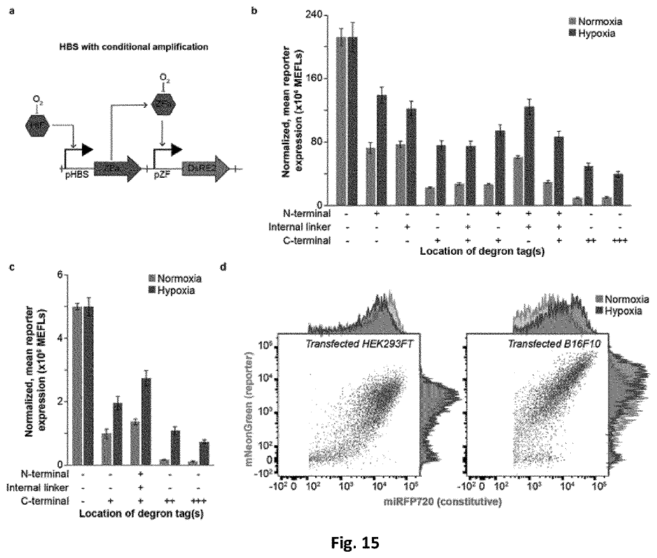

One potential concern with genetic circuits employing amplification and/or positive feedback is the risk of amplifying leaky gene expression or, in the case of positive feedback, constitutive induction of a circuit. One way to mitigate this is to develop COMET TFs that are sensitive to the presence of oxygen (i.e., that are degraded in the presence of oxygen). We tested whether appending a small oxygen-sensing amino acid motif from HIF1α onto a COMET ZFa would achieve this goal. FIG. 11 shows an annotated map (A) and DNA and protein sequences (B) of a COMET transcription factor used in the present experiments. FIG. 12 shows an annotated map (A) and DNA and protein sequences (B) of a COMET transcription factor with oxygen degradation tags on the N-terminus, in the internal linker, and on the C-terminus. FIG. 13 shows an annotated map (A) and DNA sequence (B) of a COMET promoter with DNA binding sites for a COMET transcription factor. FIG. 14 shows an annotated map of a COMET reporter plasmid with a COMET promoter producing the LumiScarlet reporter protein. FIG. 15A illustrates a schematic of an HBS utilizing an oxygen-sensitive ZFa to provide reporter amplification without inadvertent constitutive activation.

Several locations and copy number repeats for the oxygen sensitivity tags were tested in HEK293FT cells for their ability to destabilize the COMET TF in the presence of oxygen while still permitting the TF to induce gene expression in the absence of oxygen. While all variants conferred oxygen-dependent gene expression, the 2×-C-terminal repeat did so the best, meaning that this had the lowest level of reporter gene expression in the presence of oxygen while still conferring a substantial amount of gene expression in the absence of oxygen (FIG. 15B). This result was arrived at through iterative testing and design of these variants. These new oxygen-instable transcription factors also displayed conditional expression when evaluated in B16F10 cells, in a subsequent experiment where only the top performing TFs from FIG. 15B were tested (FIG. 15C). Interestingly, these two cell types showed different trends in response to hypoxia—while the transiently transfected HEK293FT cells showed slightly lower expression of a constitutive gene introduced in the same manner as the HBS circuit, the B16F10 cells showed increased expression levels after hypoxia culture (FIG. 15D). One possible explanation is that the B16F10 cells do not proliferate as rapidly under hypoxic conditions and are therefore accumulating protein that is not diluted out upon cell division. This is possibly an artifact of transfection, as this was not observed in similar experiments with the B16F10-LP line, where the cargo is stably integrated into the genome. The use of oxygen sensitive COMET TFs, as in this Example, enables the sensing of hypoxia without relying on sensing HIF levels, potentially allowing avoiding the differential dysregulation between cell types, and producing a more robust biosensor with broad applications.

In summary, COMET TFs are amenable to oxygen-mediated degradation, and this property confers beneficial effects when an oxygen-sensitive COMET TF is used as a stand-alone HBS and could confer beneficial effects when used to modulate the signal from a DNA-based HBS.

### Example 6—Hypoxia Biosensor Performance can be Enhanced with COMET Transcription Factors

The present Example illustrates a class of enhanced hypoxia biosensors using COMET (Composable Mammalian Elements of Transcription) transcription factors.

While there are many ways in which this could be possible, four have been tested. Many more iterations of ways to enhance hypoxia biosensors and biosensors in general with the COMET platform are likely possible. The four circuits that have been designed and tested so far are as follows:

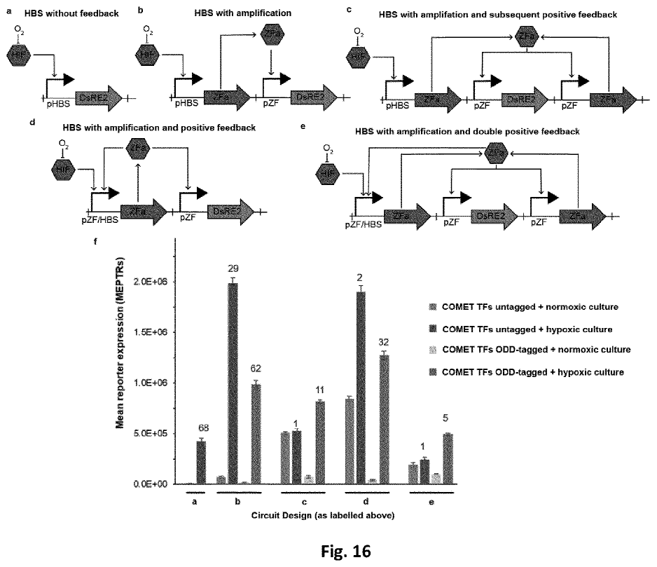

For reference, a hypoxia biosensor that is not enhanced with feedback or amplification elements is shown in FIG. 16A. A hypoxia biosensor enhanced by signal amplification by a COMET transcription factor is shown in FIG. 16B, 18. The hypoxia biosensor produces a COMET transcription factor, which then activates expression of a protein from a separate locus. This circuit enhances the hypoxia biosensor regardless of whether the COMET transcription factor has been modified to be destabilized in the presence of oxygen or not (See FIG. 16F).

A hypoxia biosensor enhanced by signal amplification by a COMET transcription factor, with subsequent positive feedback, is shown in FIG. 16C, 19. The hypoxia biosensor produces a COMET transcription factor, which then activates expression of a protein from a separate locus. The COMET transcription factor also activates expression of more of itself from a separate transcription unit. This circuit does not appear to enhance the hypoxia biosensor without the modifications to destabilize the COMET transcription factor in the presence of oxygen (See FIG. 16F).

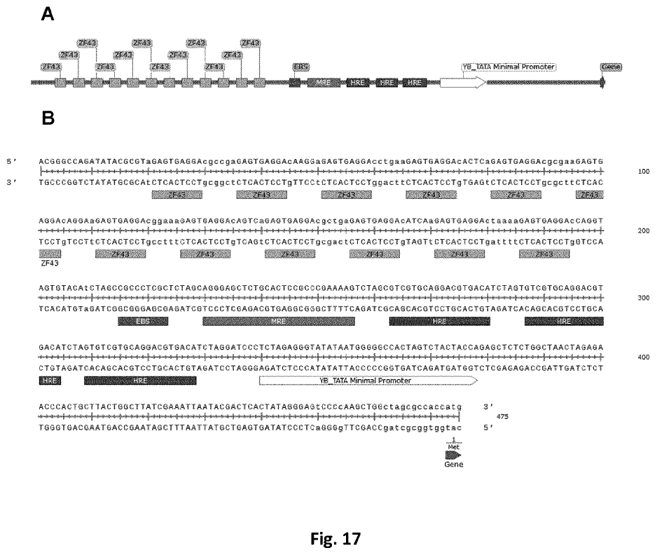

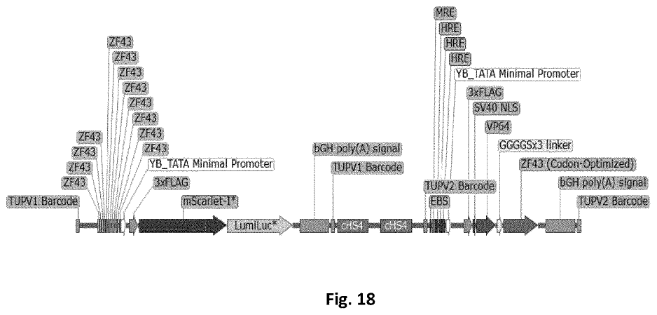

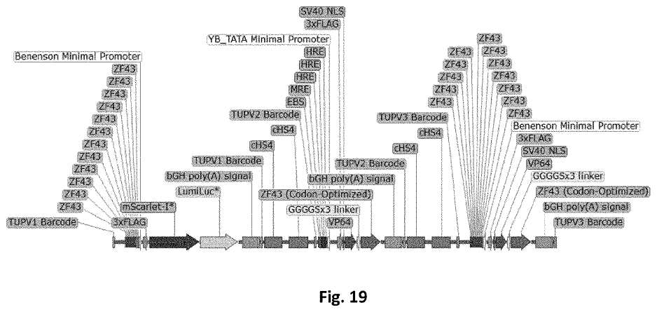

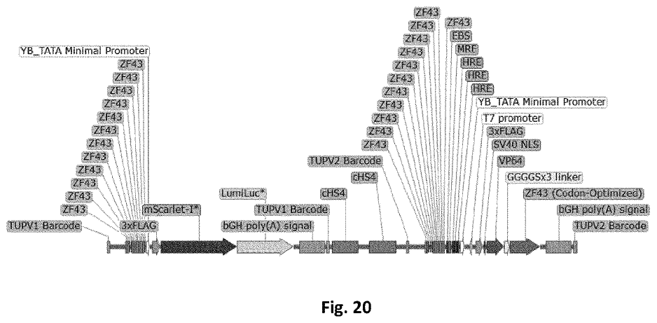

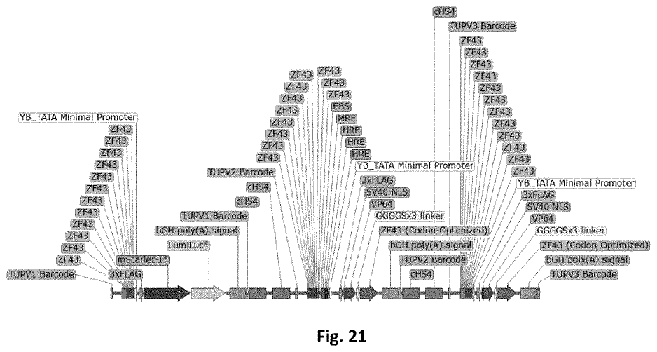

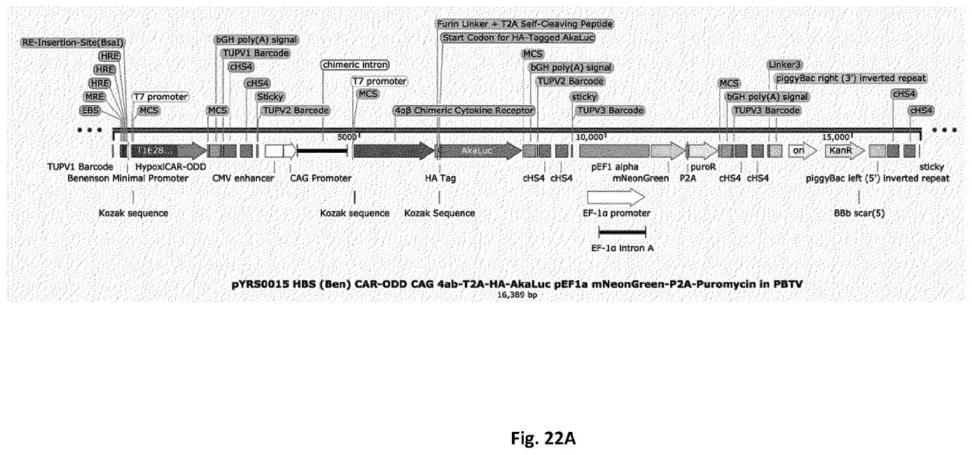

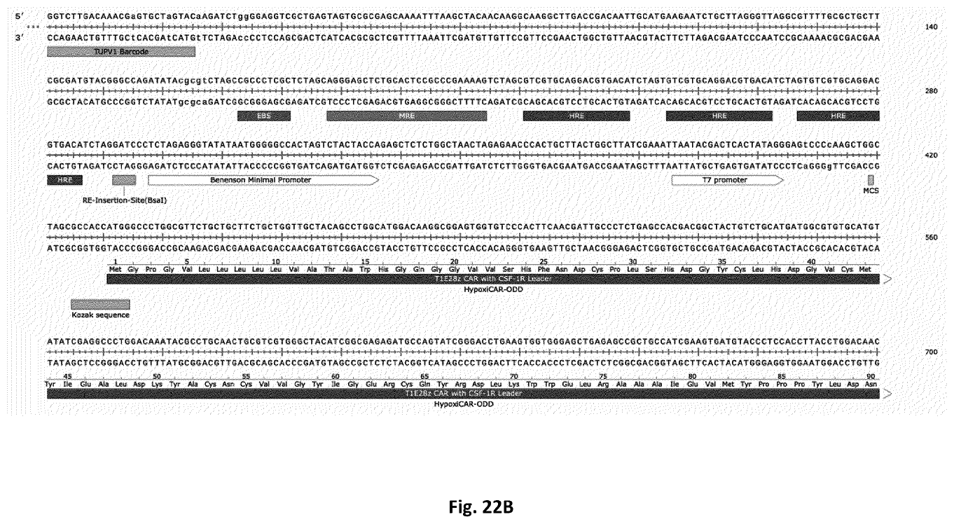

A hypoxia biosensor enhanced by signal amplification by a COMET transcription factor, with positive feedback, is shown in FIG. 16D, 20. The hypoxia biosensor produces a COMET transcription factor, which then activates expression of a protein from a separate locus. The COMET transcription factor also induces expression of more of itself by binding to a locus upstream of the hypoxia biosensor (See FIG. 17, which illustrates an annotated map (A) and DNA sequence (B) of a hypoxia biosensor with upstream binding sites for a COMET transcription factor). This circuit enhances the hypoxia biosensor regardless of whether the COMET transcription factor has been modified to be destabilized in the presence of oxygen (See FIG. 16F); however, it performs better with the oxygen sensitive COMET transcription factor than with the unmodified COMET transcription factor.

A hypoxia biosensor enhanced by signal amplification by a COMET transcription factor, with subsequent double positive feedback is shown in FIG. 16E, 21. The hypoxia biosensor produces a COMET transcription factor, which then activates expression of a protein from a separate locus. The COMET transcription factor also activates expression of more of itself from a separate transcription unit. The COMET transcription factor also induces expression of more of itself by binding to a locus upstream of the hypoxia biosensor (See FIG. 17). This circuit does not appear to enhance the hypoxia biosensor without the modifications to destabilize the COMET transcription factor in the presence of oxygen (See FIG. 16F).

As shown in FIG. 16F, B16F10 cells with hypoxia biosensors with or without several feedback strategies integrated into their genomes were cultured under normoxic or hypoxic conditions and reporter expression analyzed by flow cytometry. The x-axis labels denote which of the hypoxia biosensor enhancement strategies demonstrated in panels A through E were integrated into the cell line tested. For some cell lines, the COMET transcription factors were tagged with oxygen-degradation domains (ODDs) as described supra. Each cell line tested contained a hypoxia biosensor with the YB_TATA minimal promoter. Fold induction is shown over the hypoxic bar. Several designs tested increased the level of hypoxia-induced gene expression from the hypoxia biosensor. The addition of an ODD tag to the COMET transcription factor consistently reduced the gene expression under normoxic conditions more so than it reduced the gene expression under hypoxic conditions, across all designs.

### Example 7—Regulation of Expression of Chimeric Antigen Receptor (CAR) by Hypoxia Biosensors

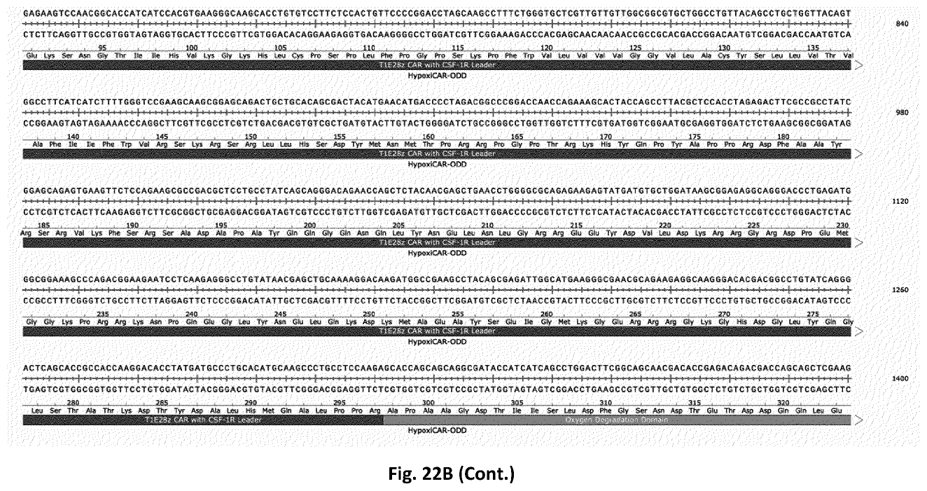

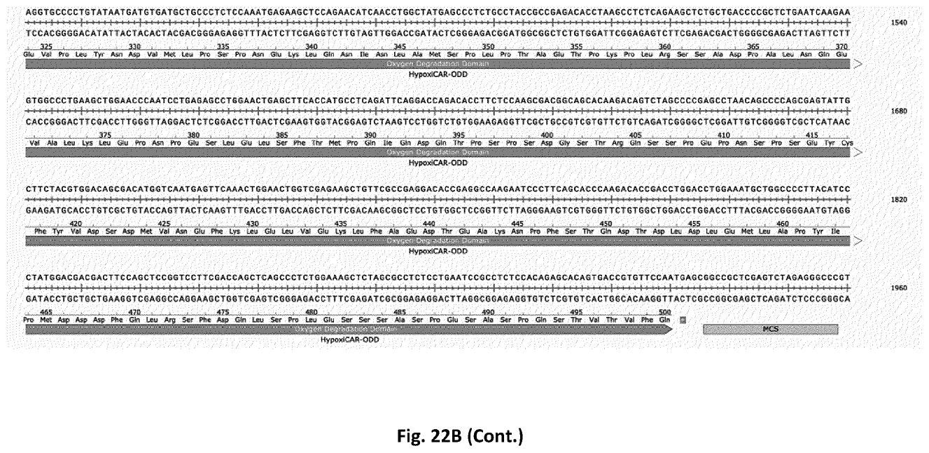

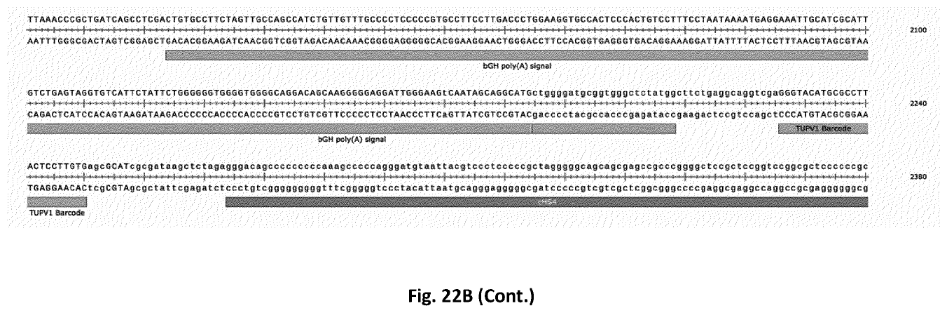

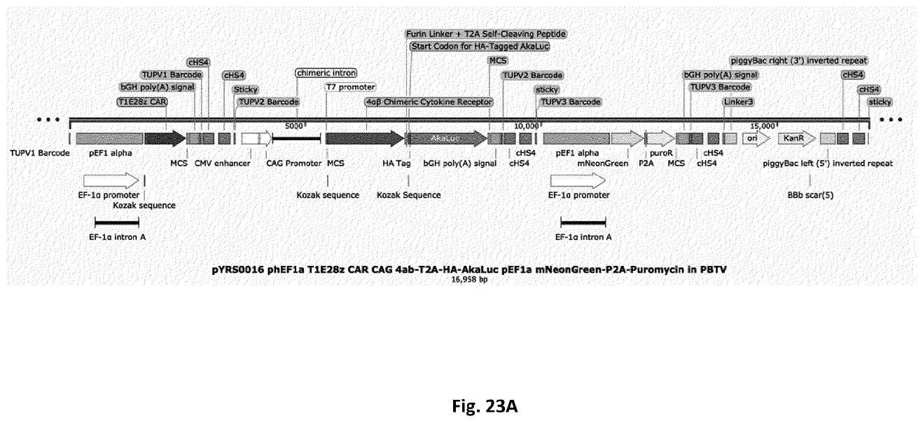

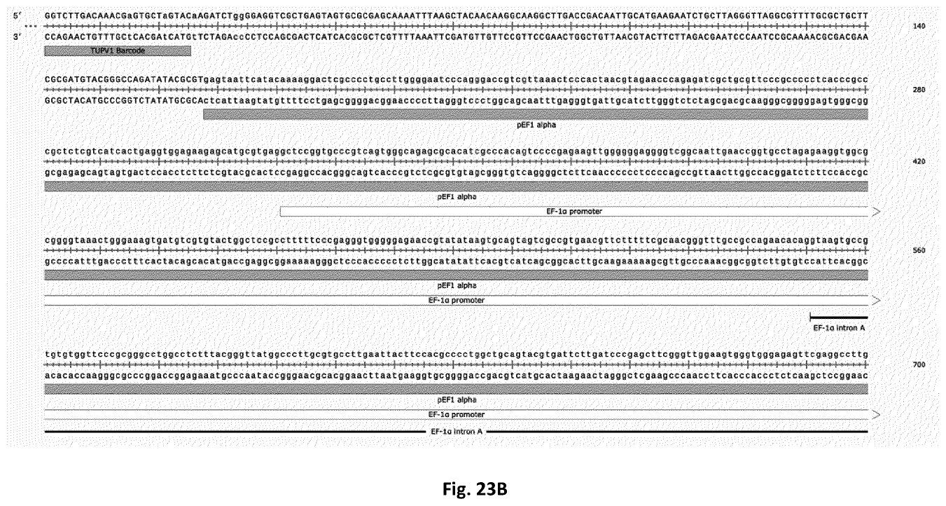

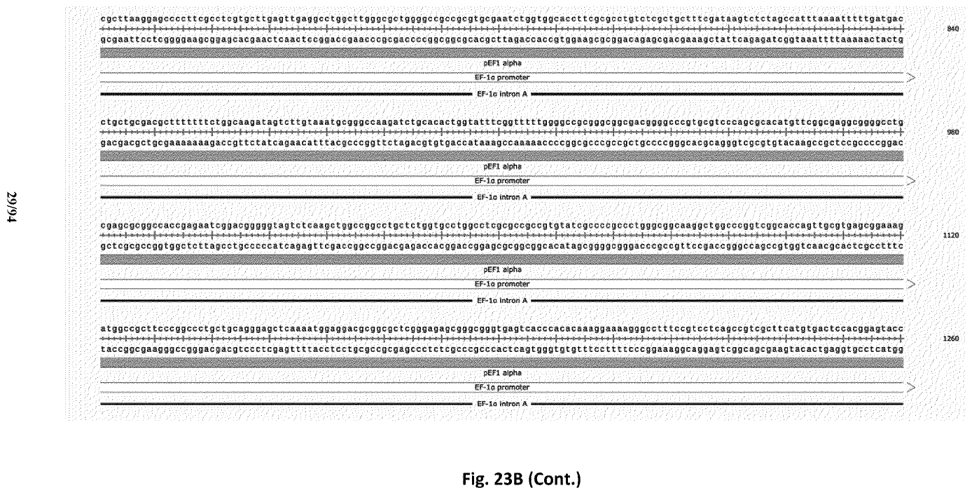

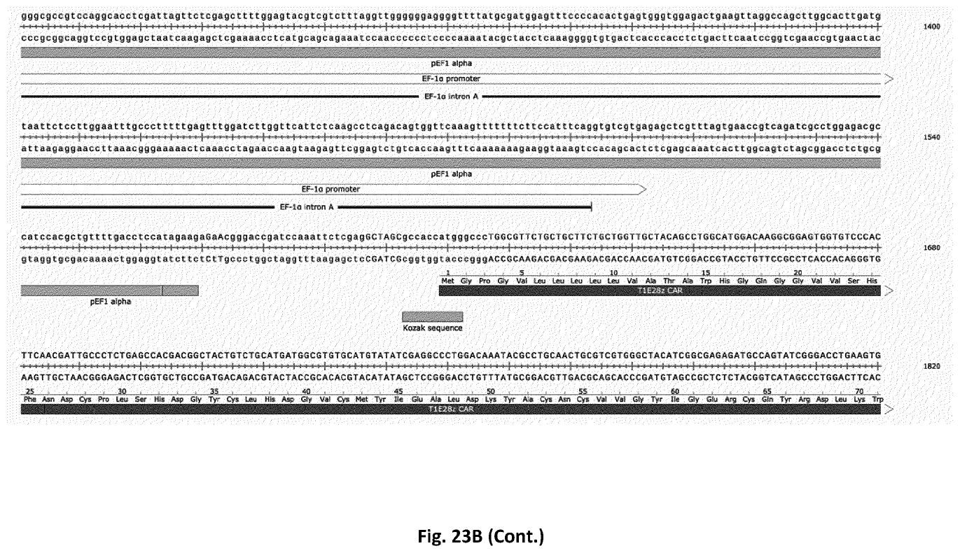

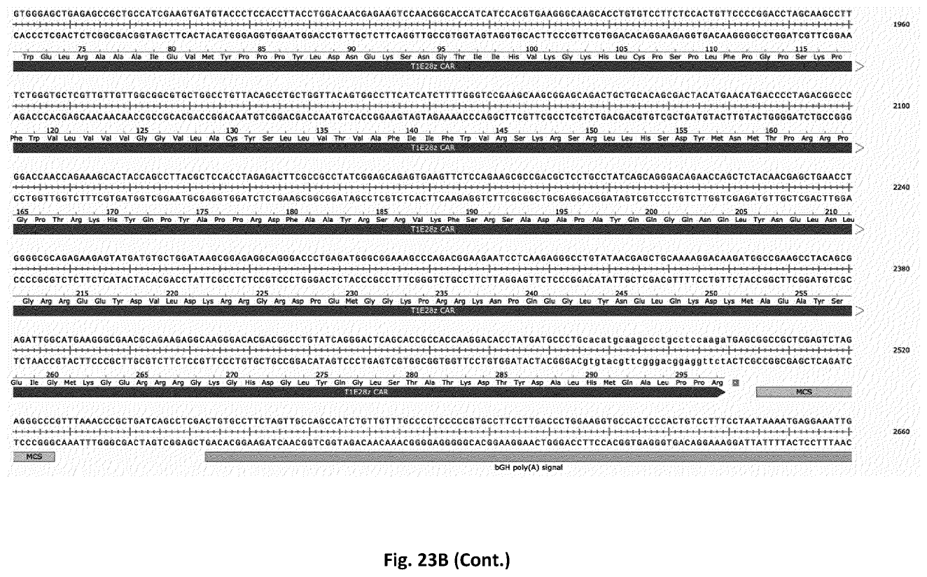

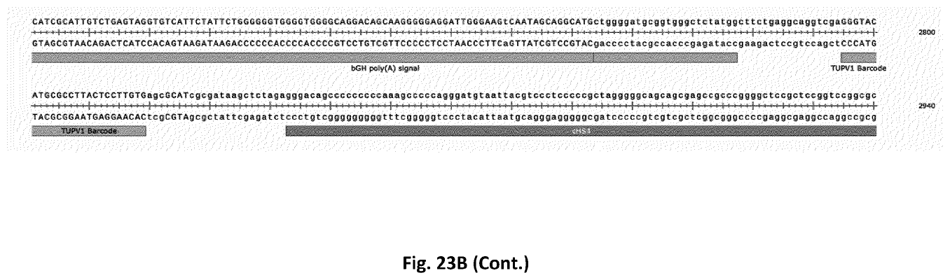

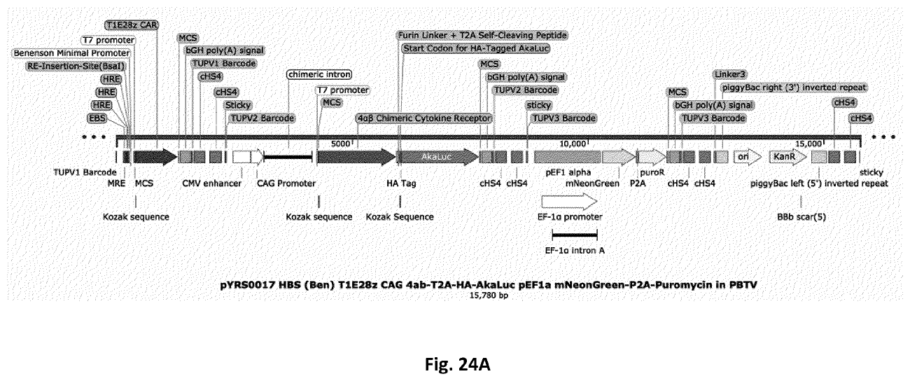

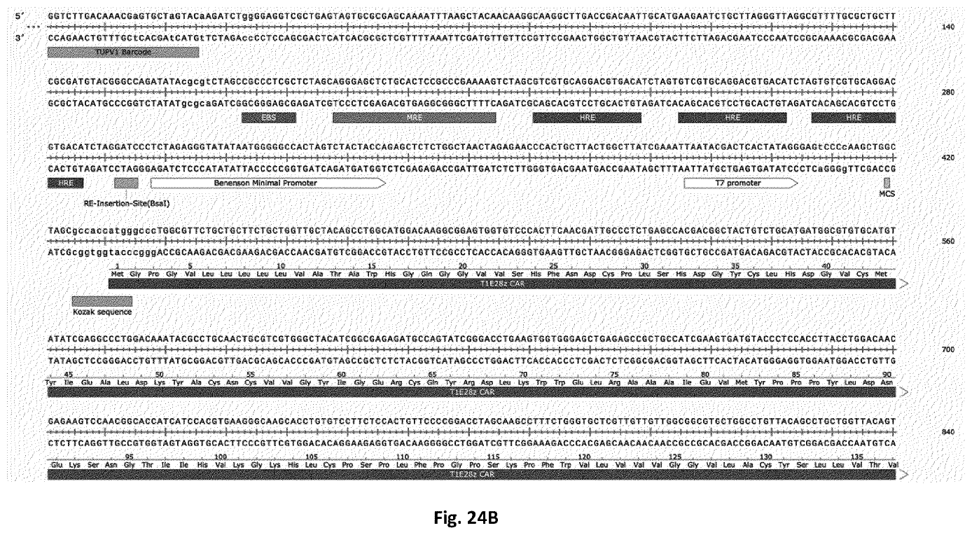

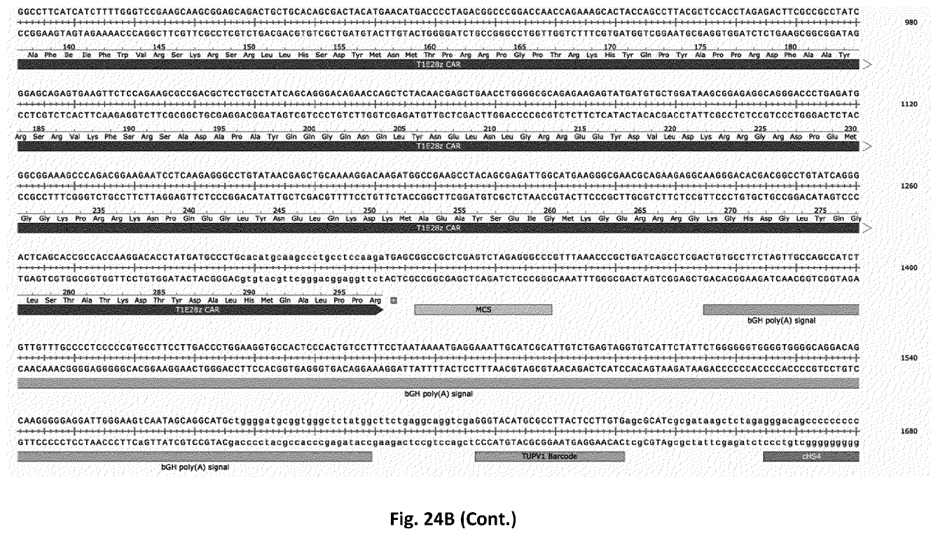

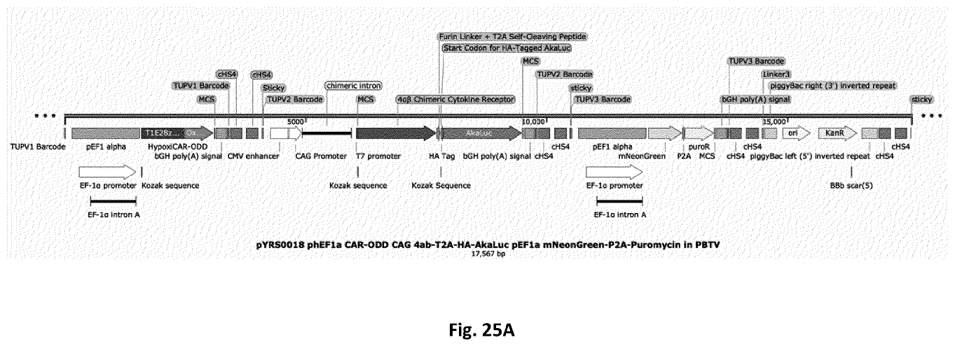

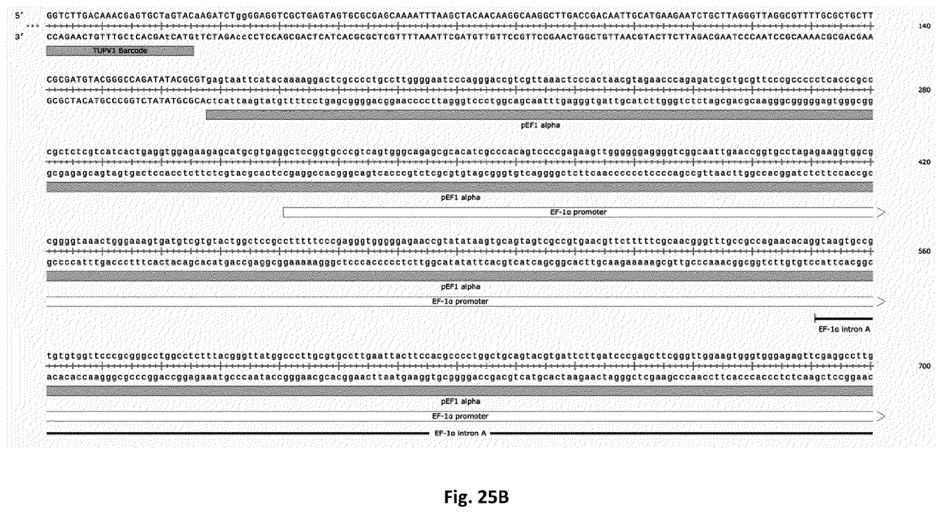

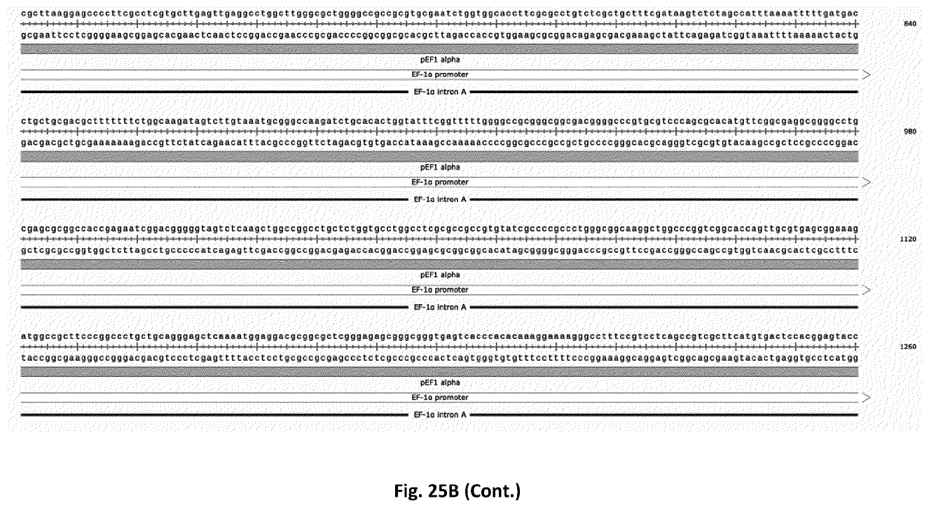

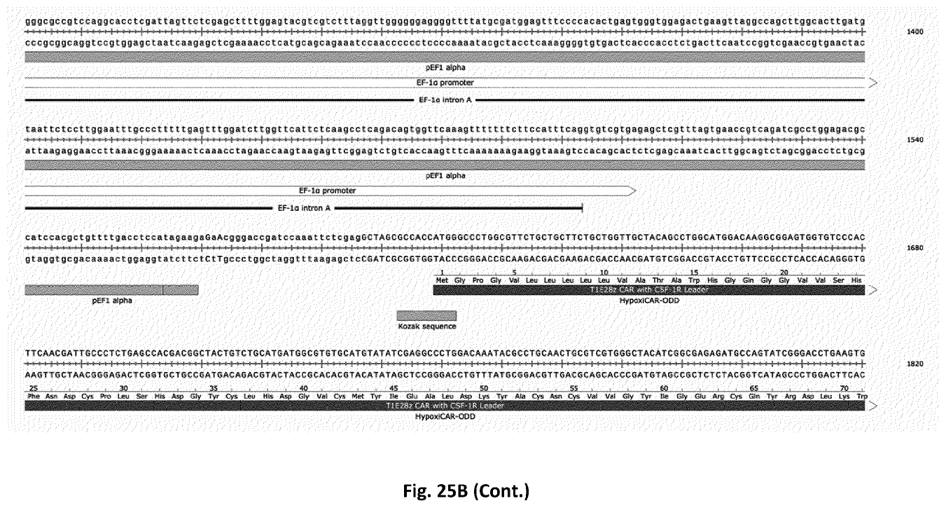

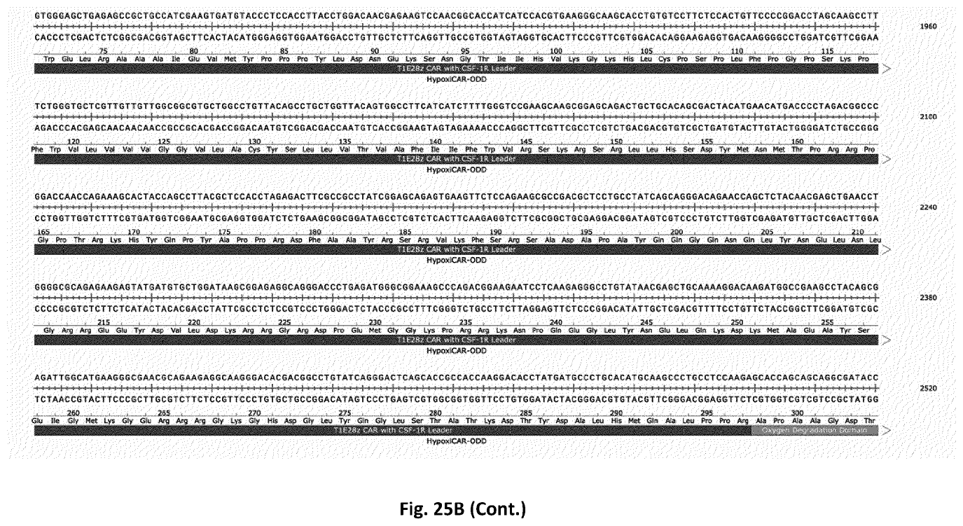

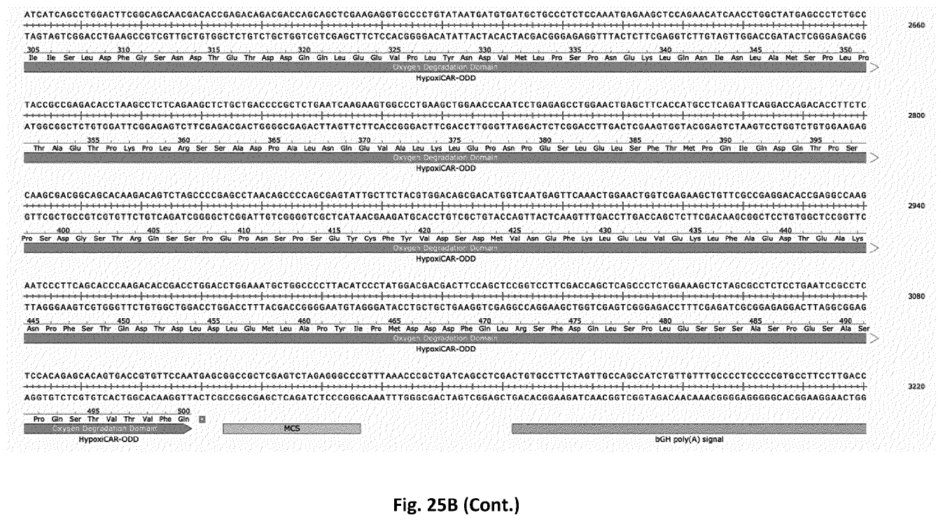

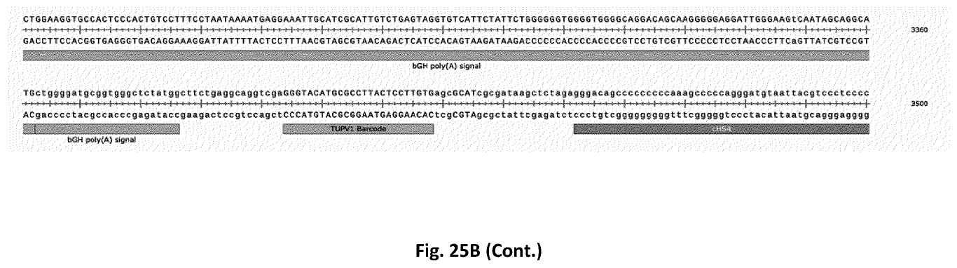

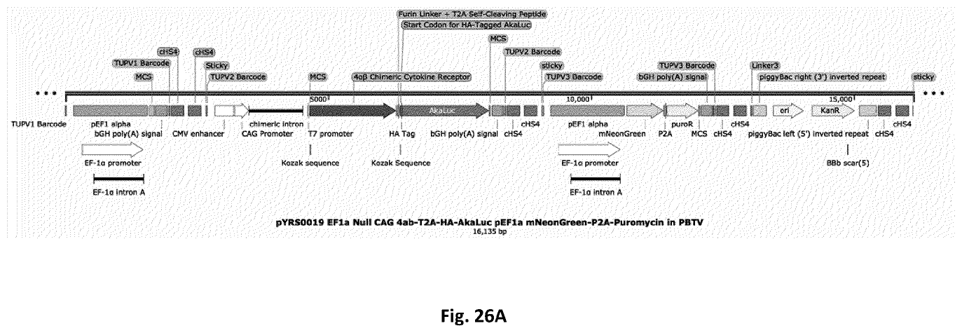

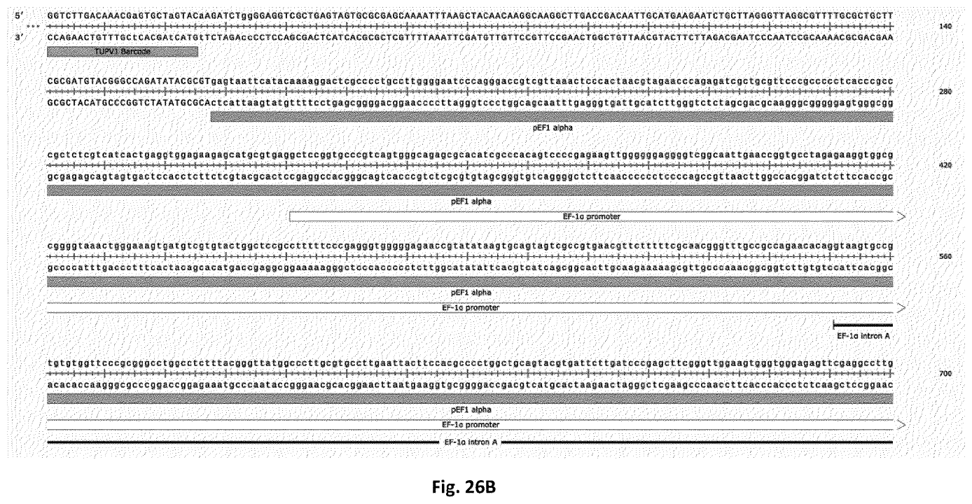

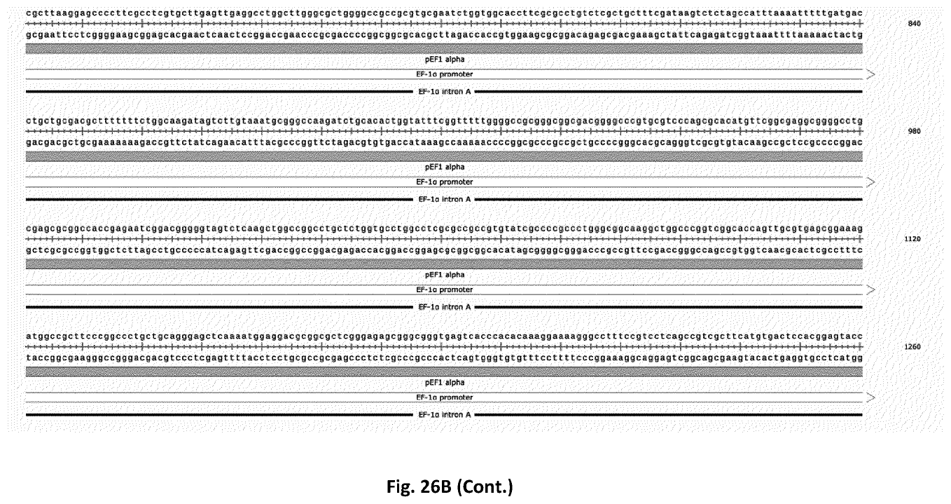

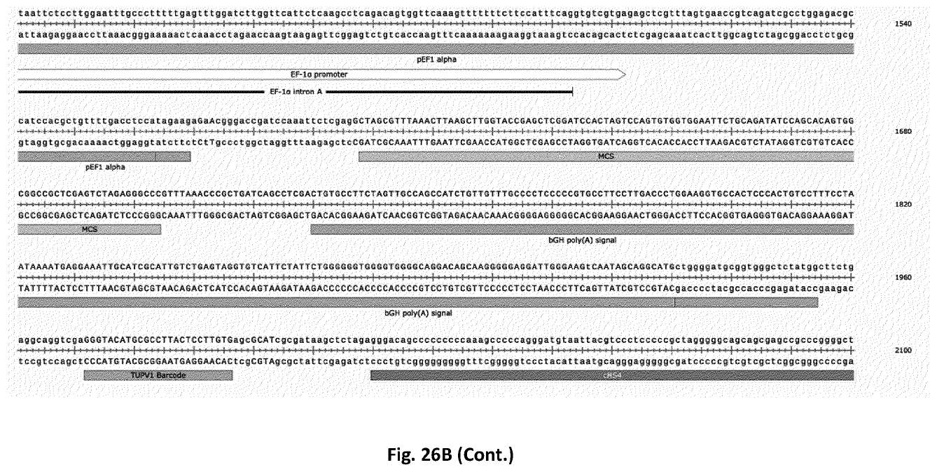

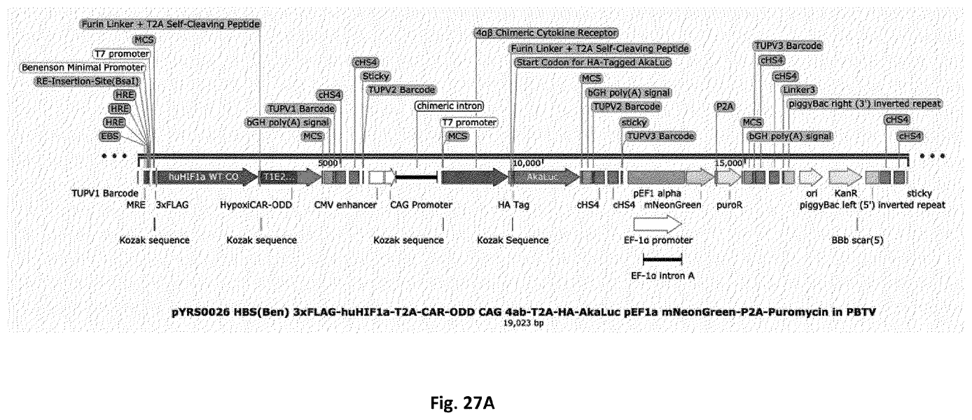

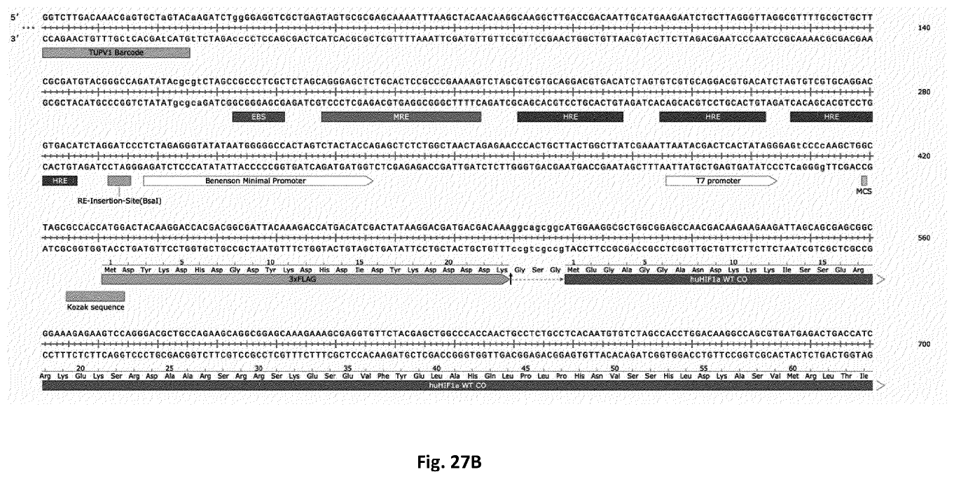

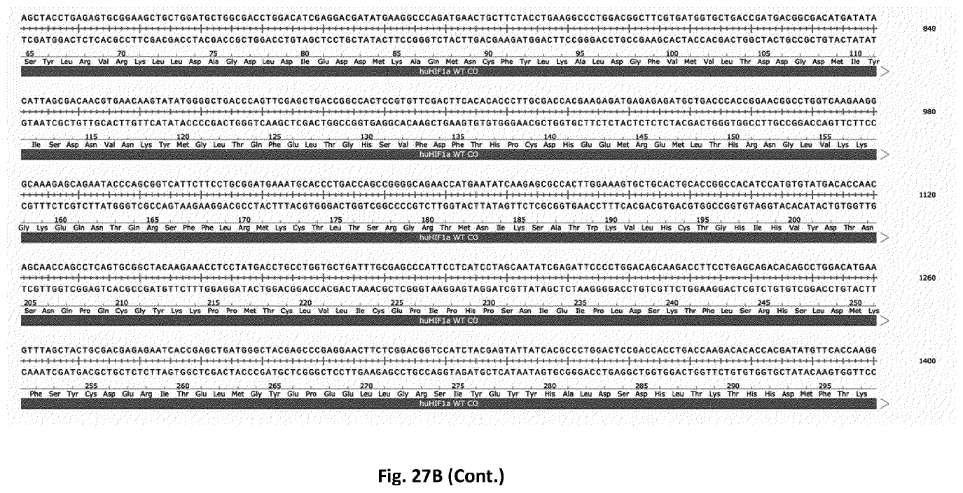

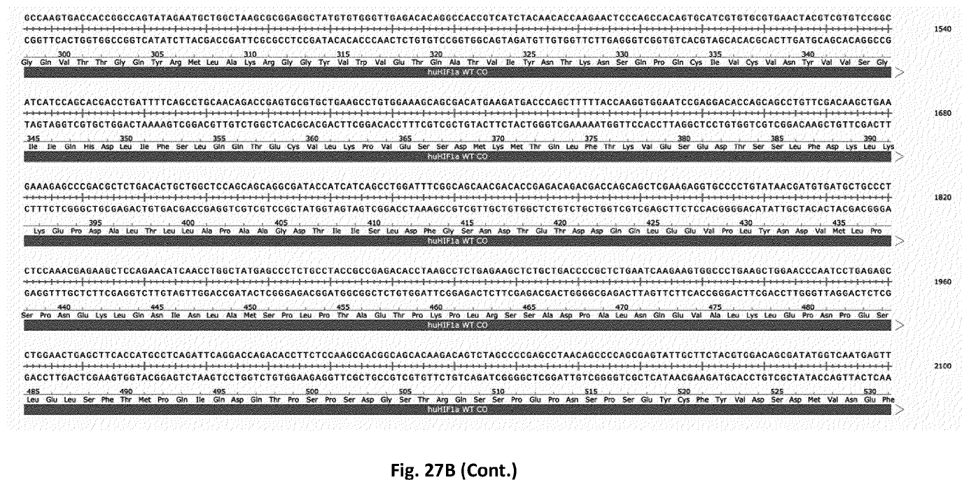

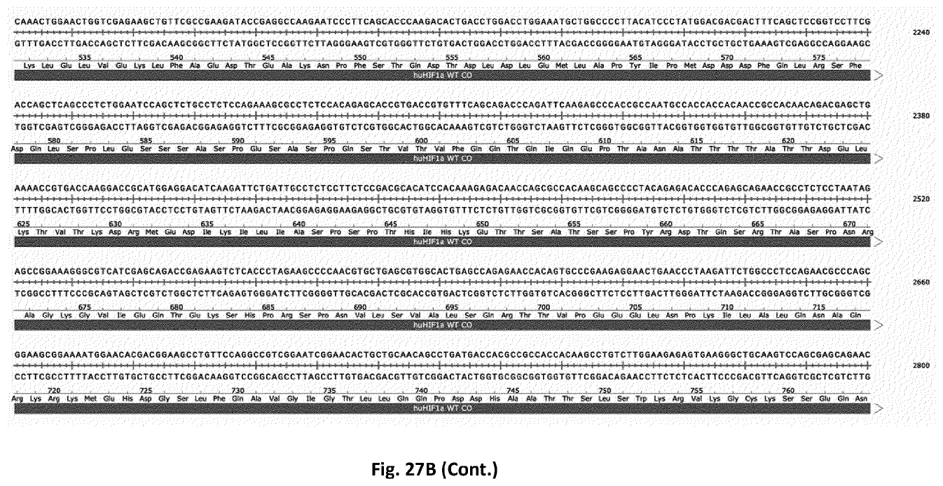

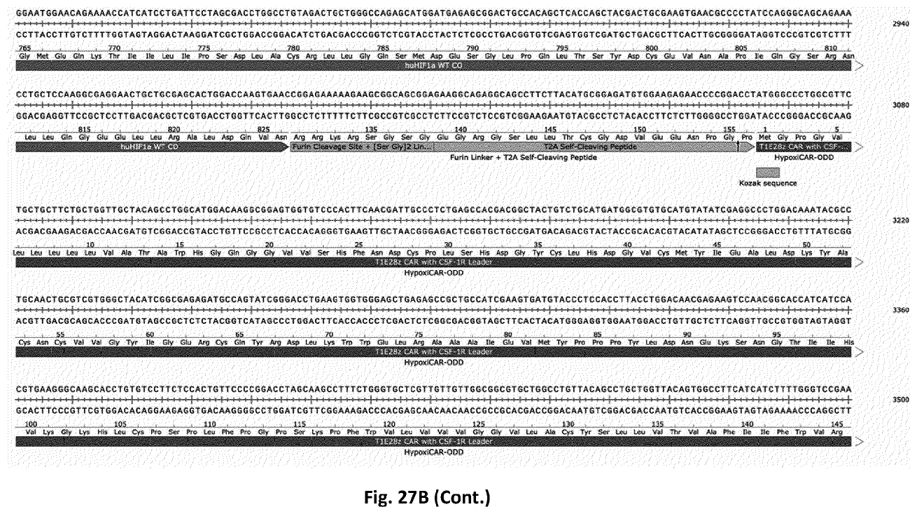

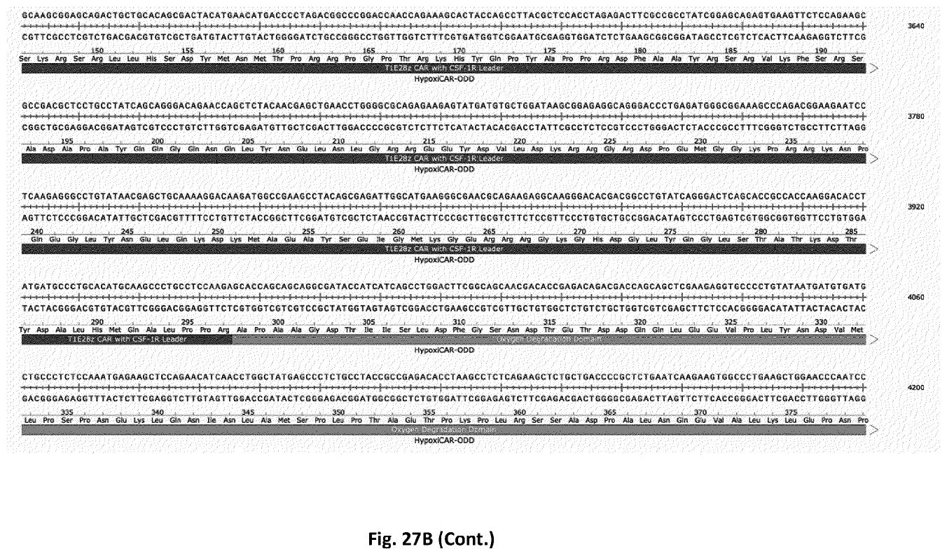

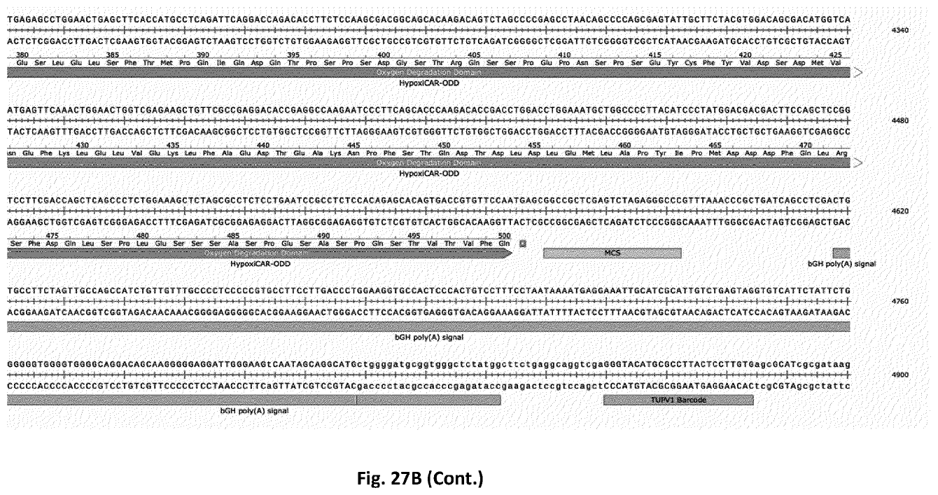

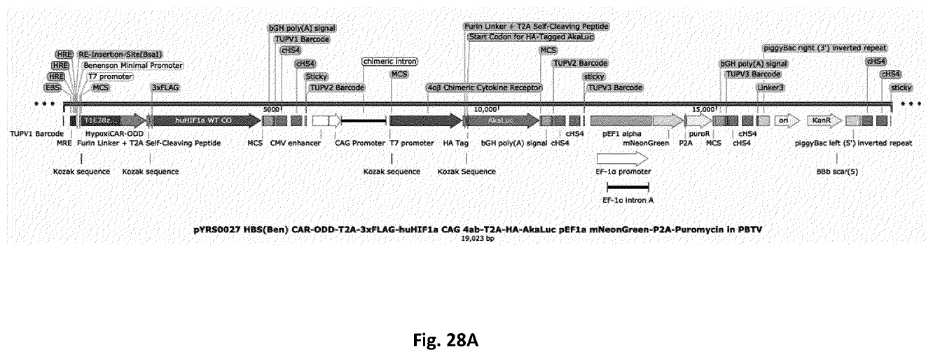

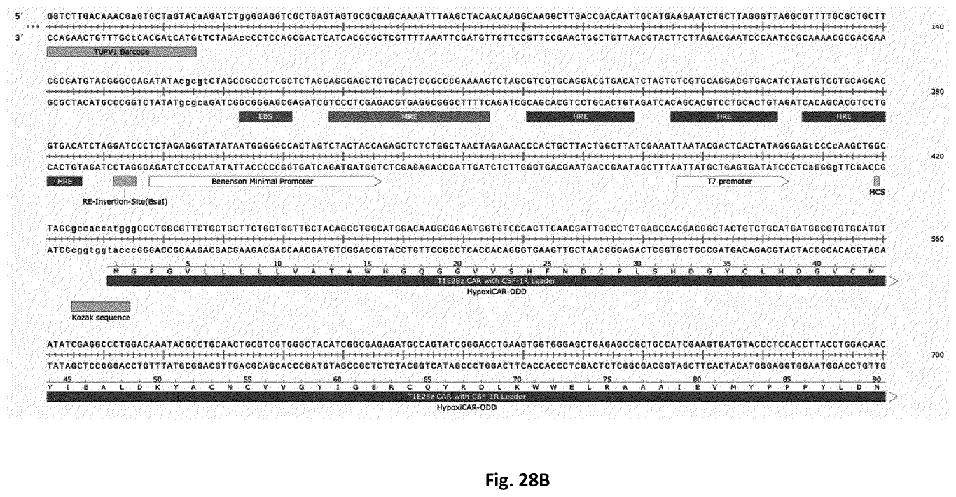

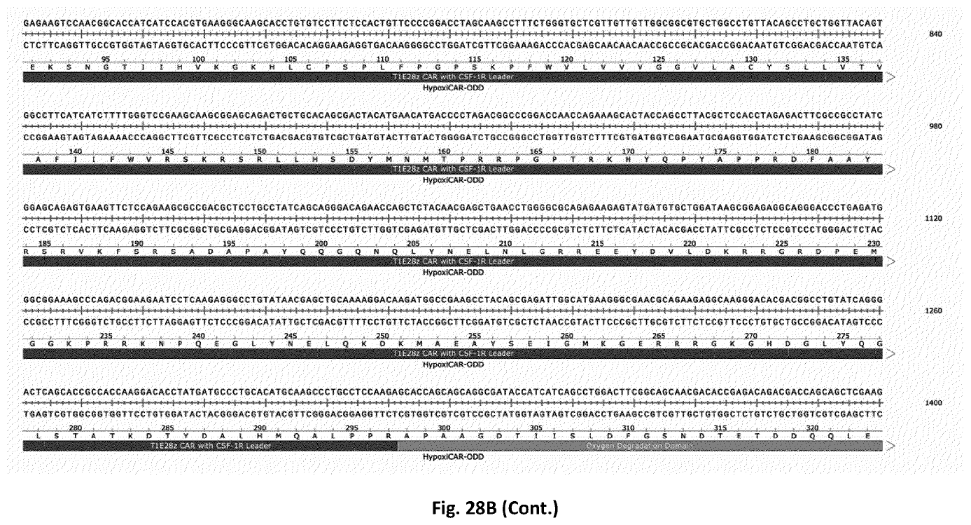

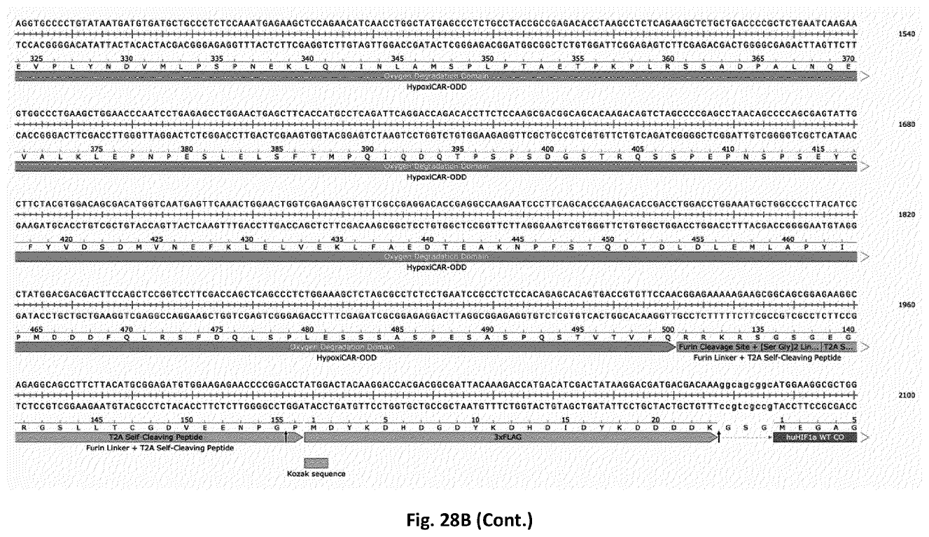

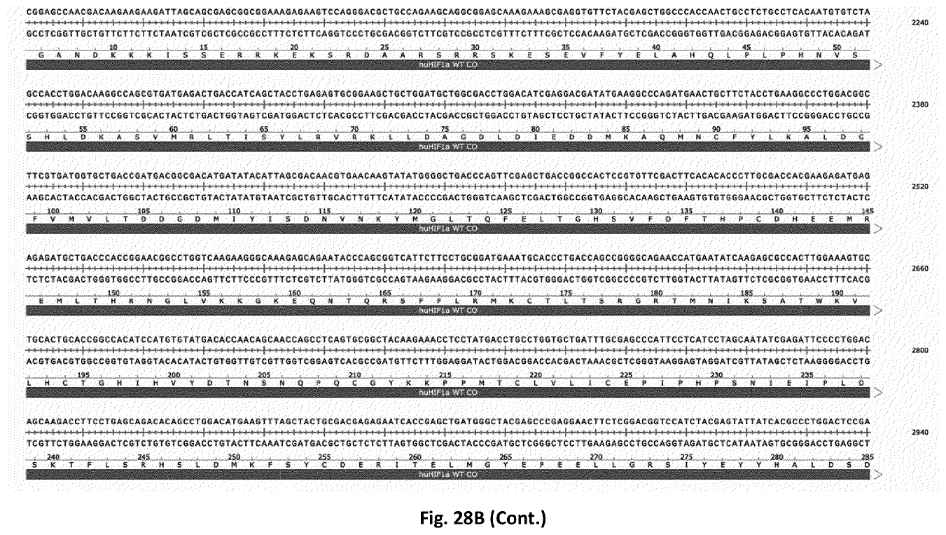

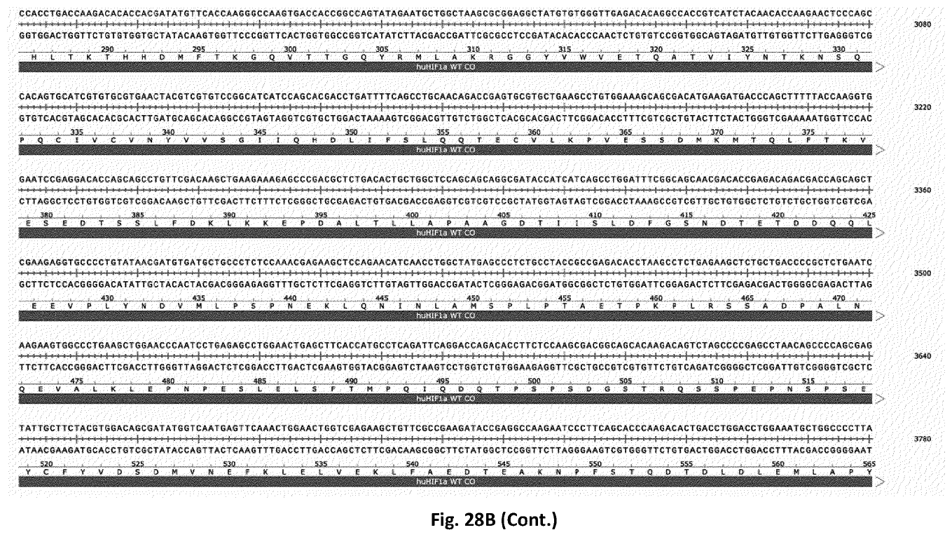

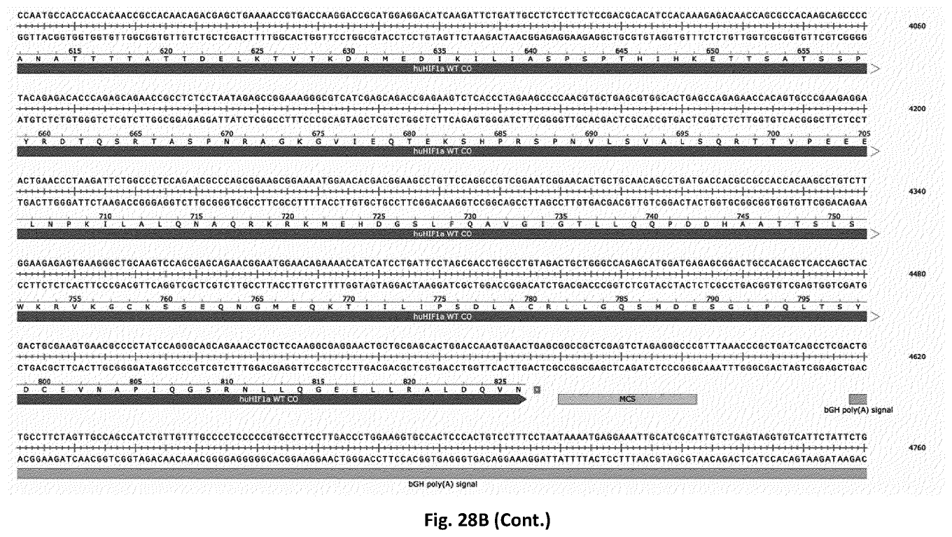

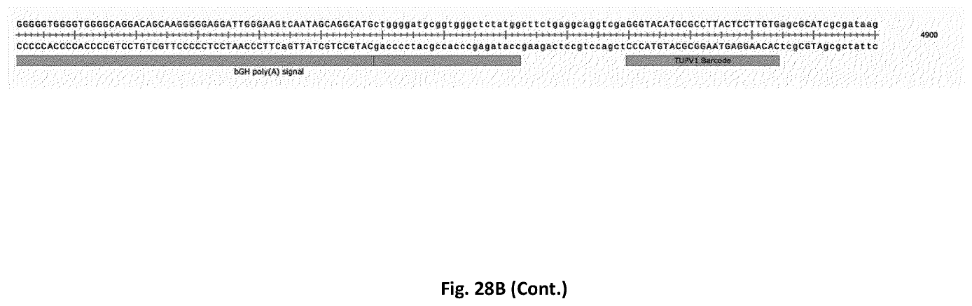

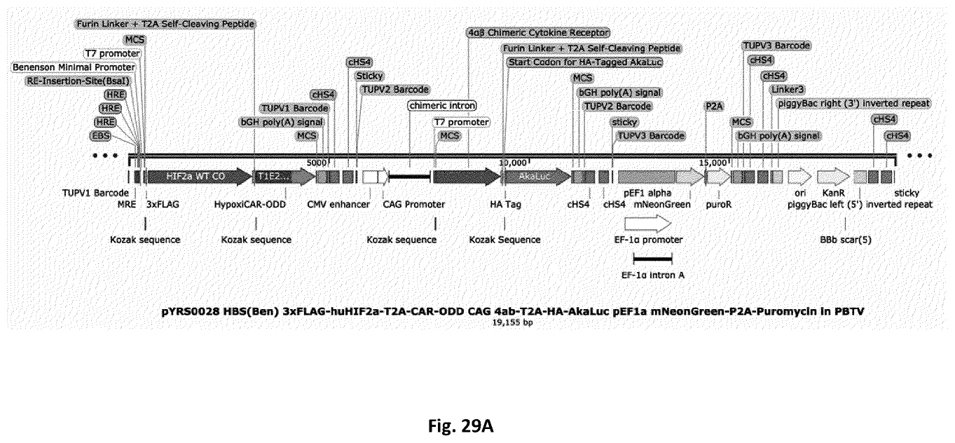

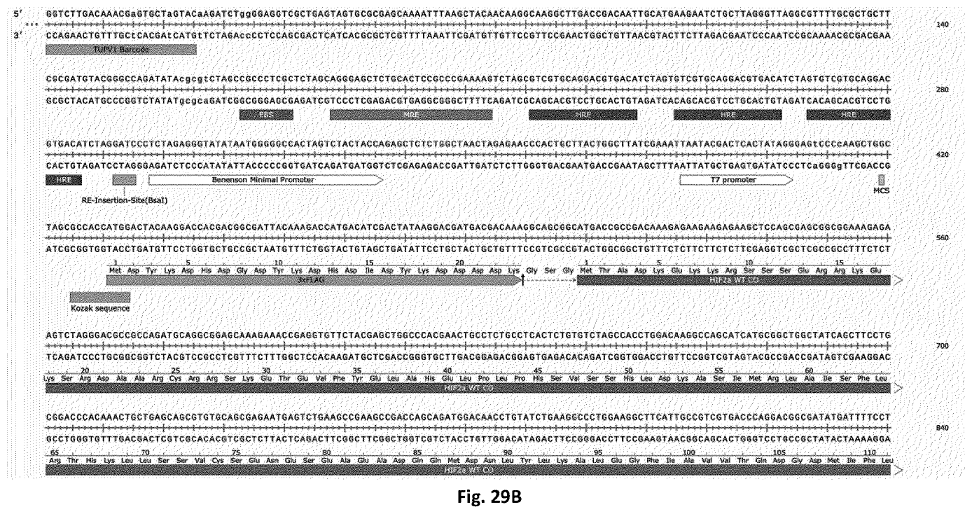

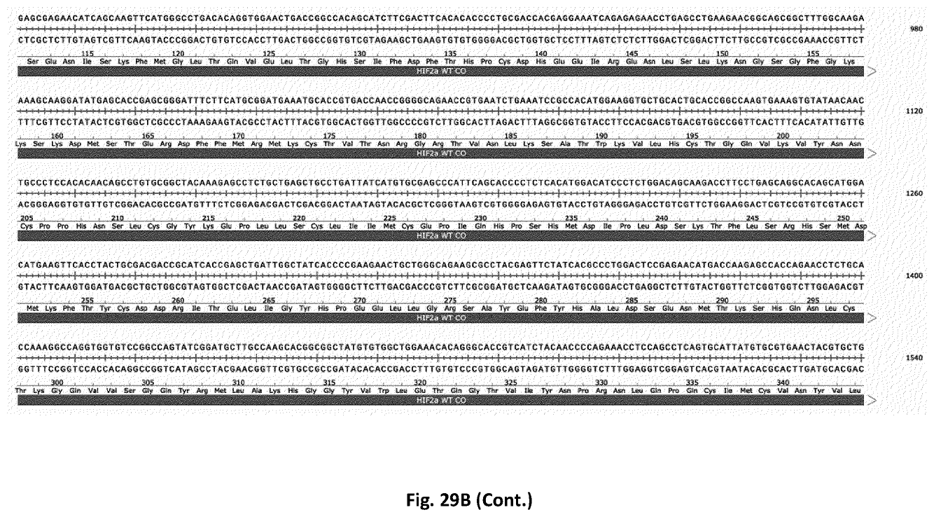

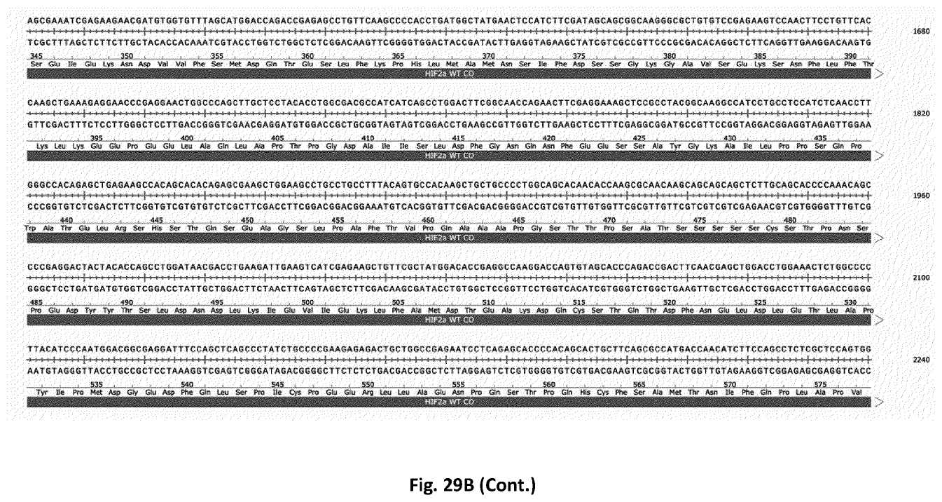

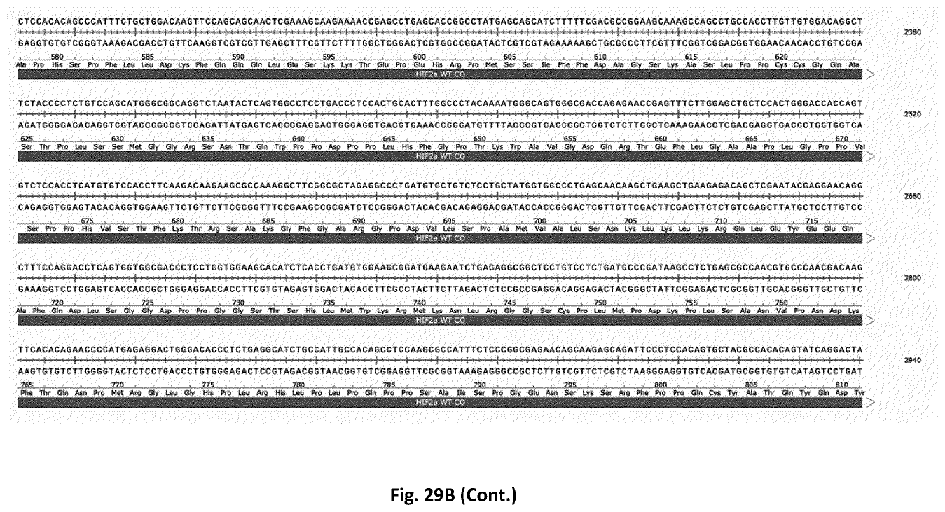

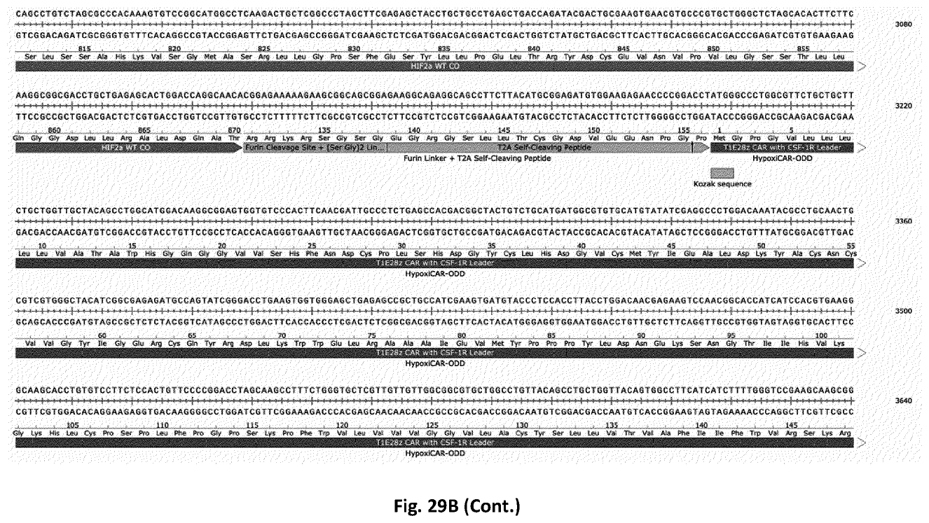

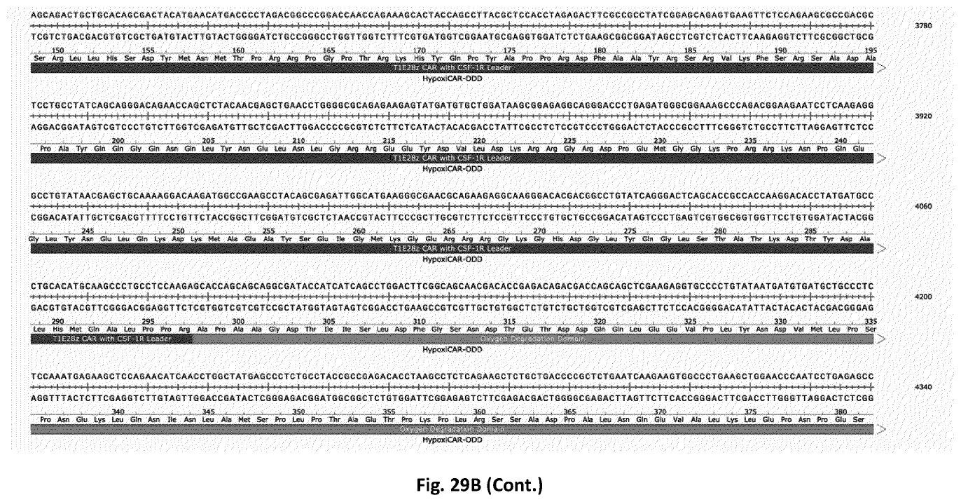

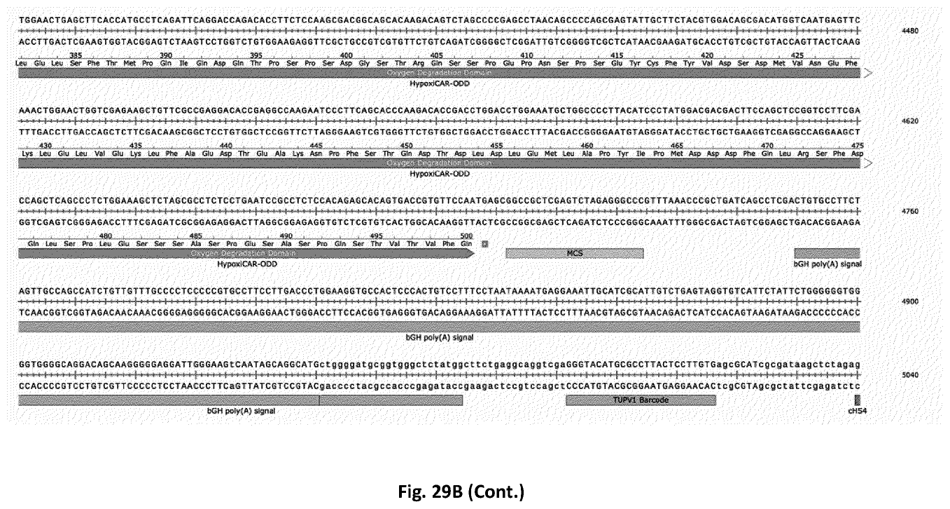

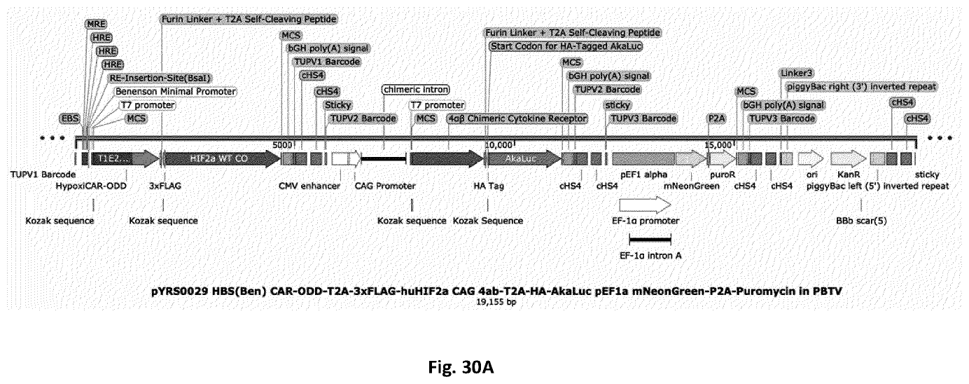

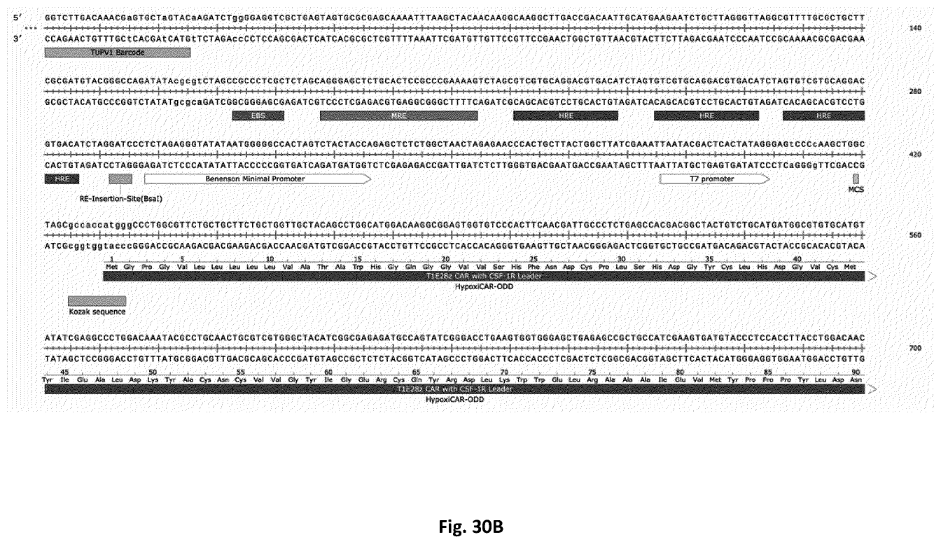

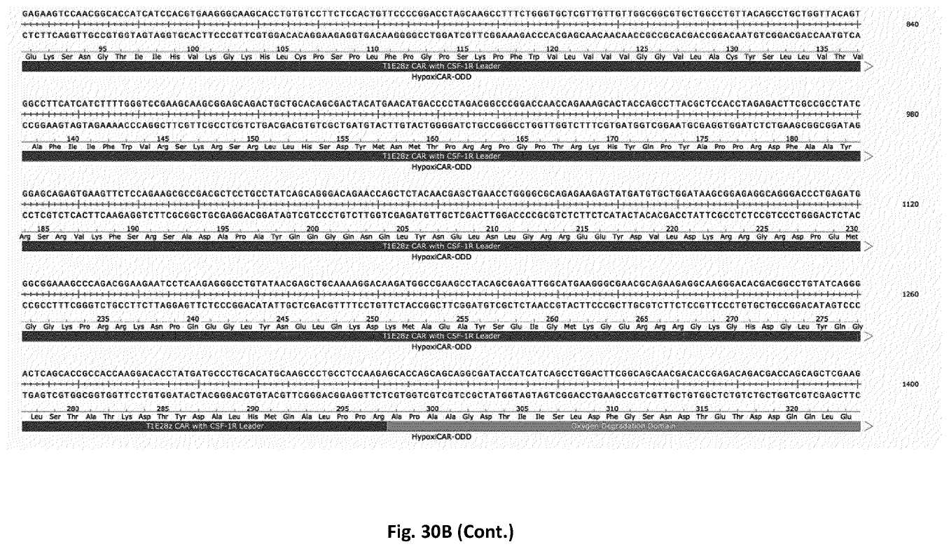

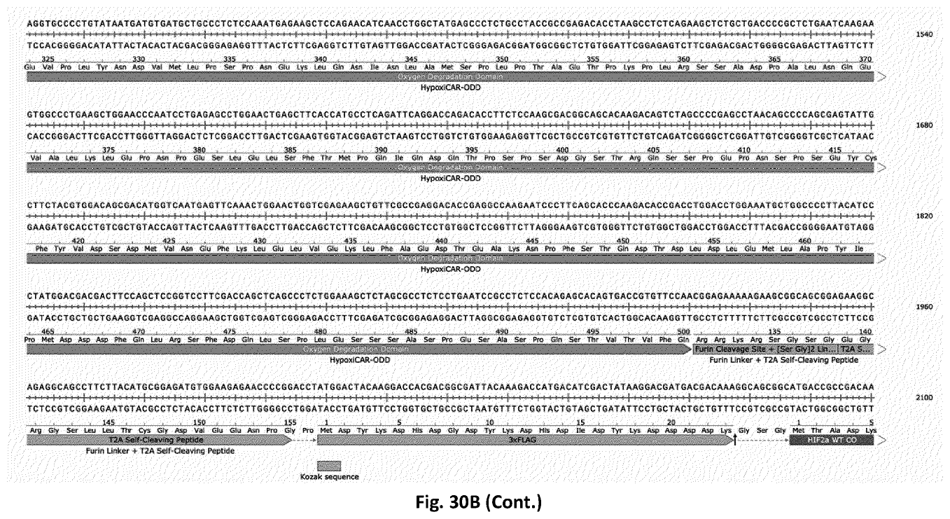

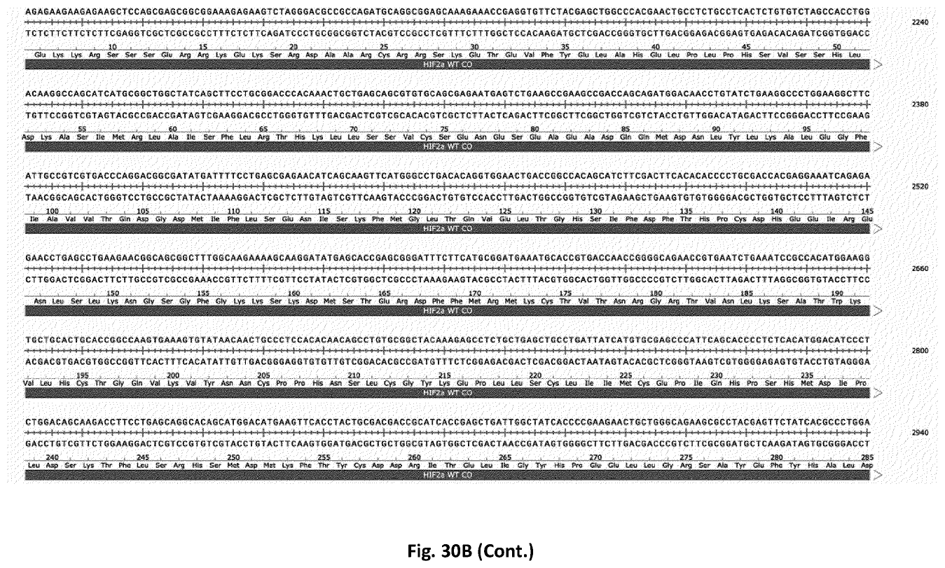

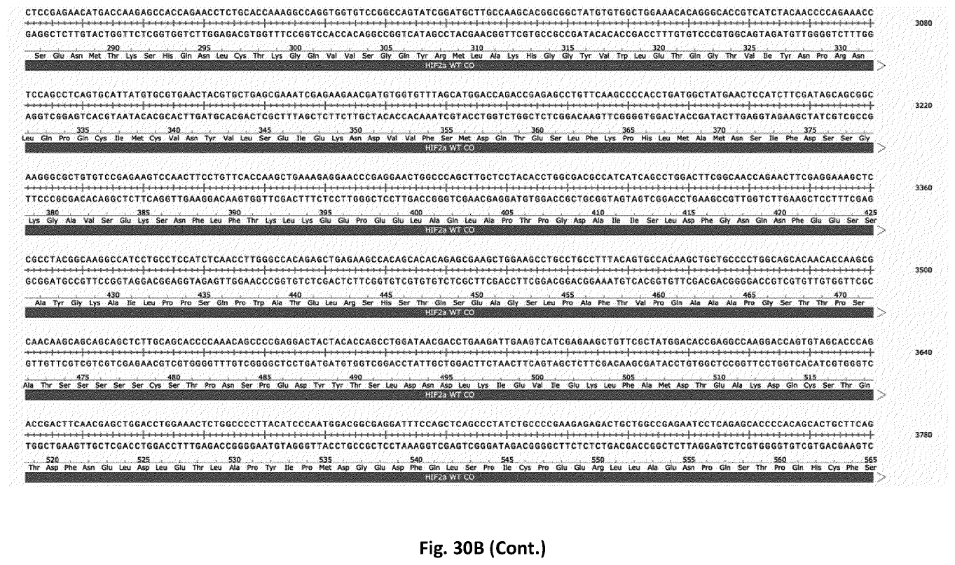

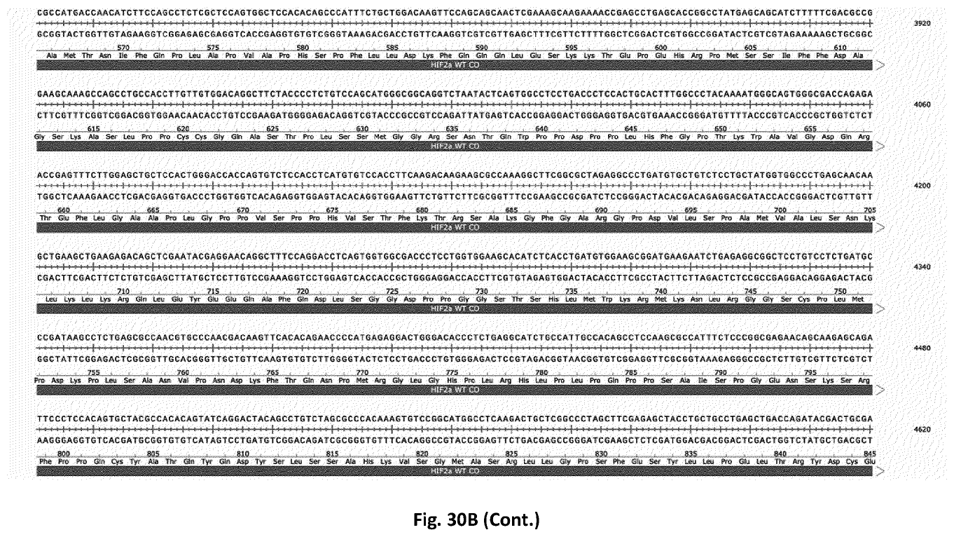

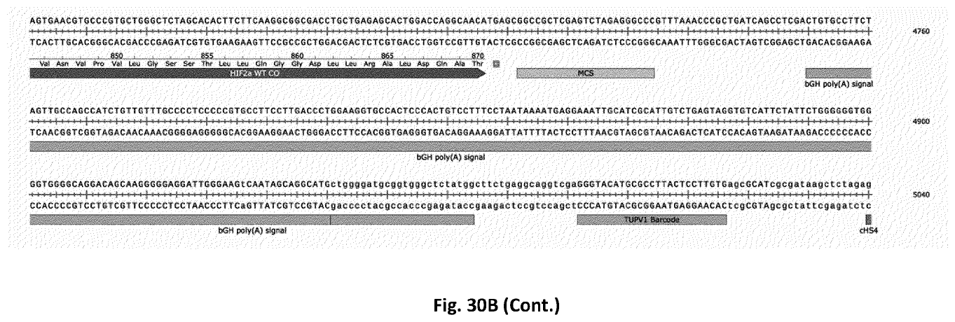

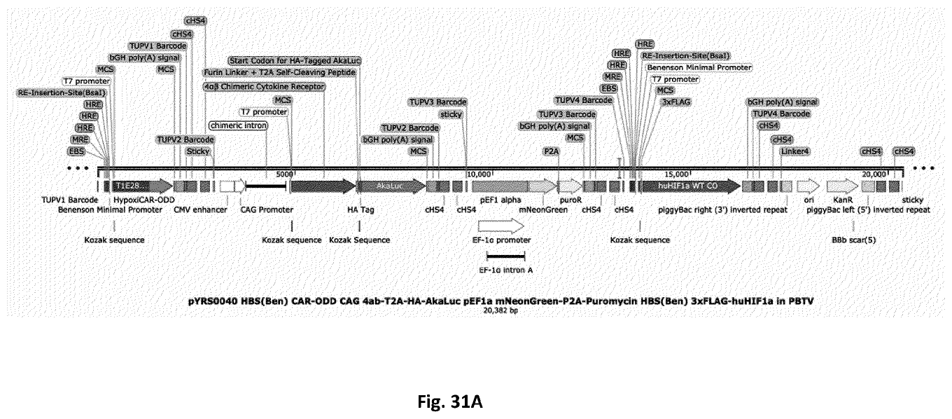

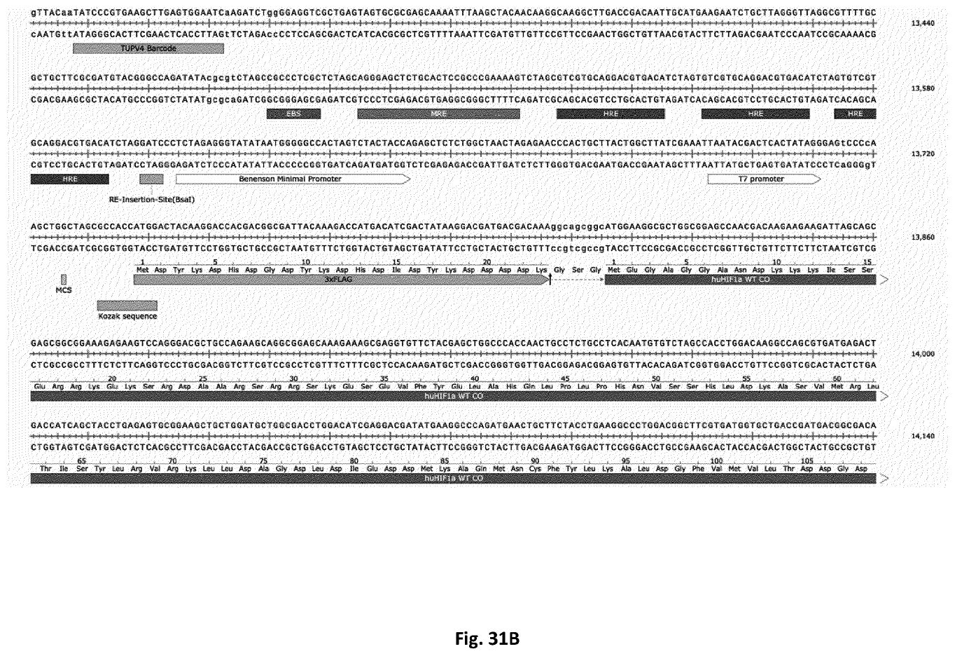

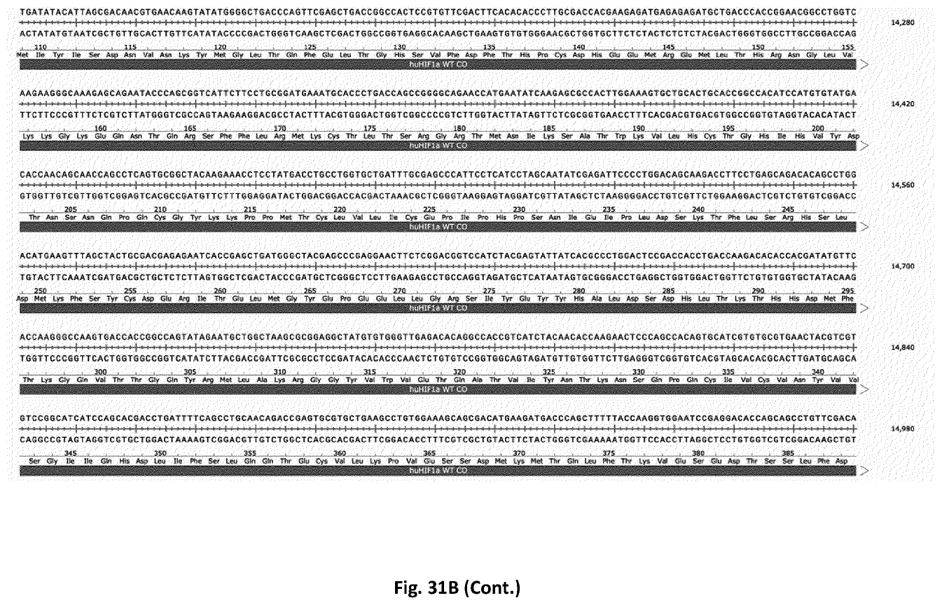

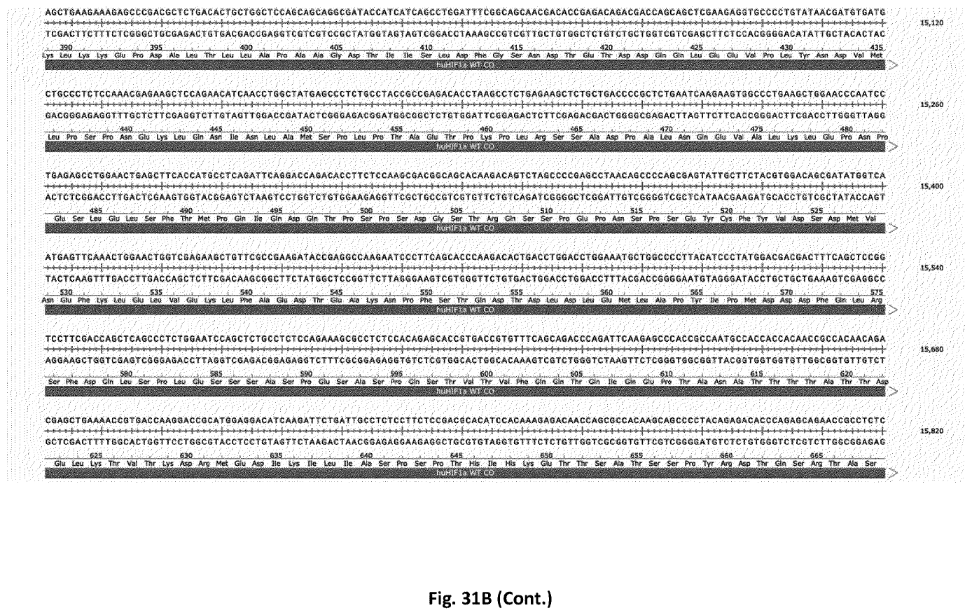

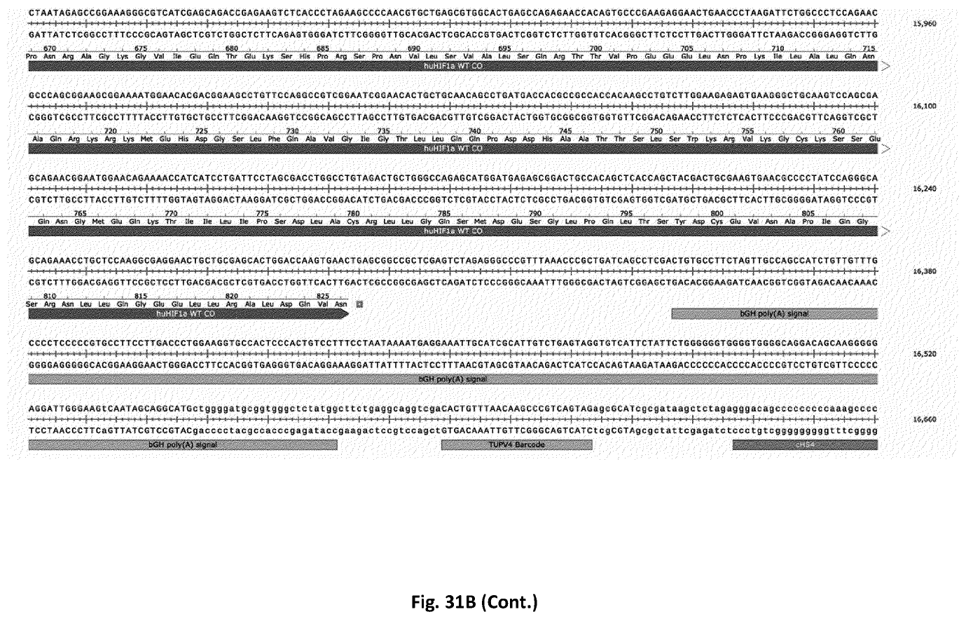

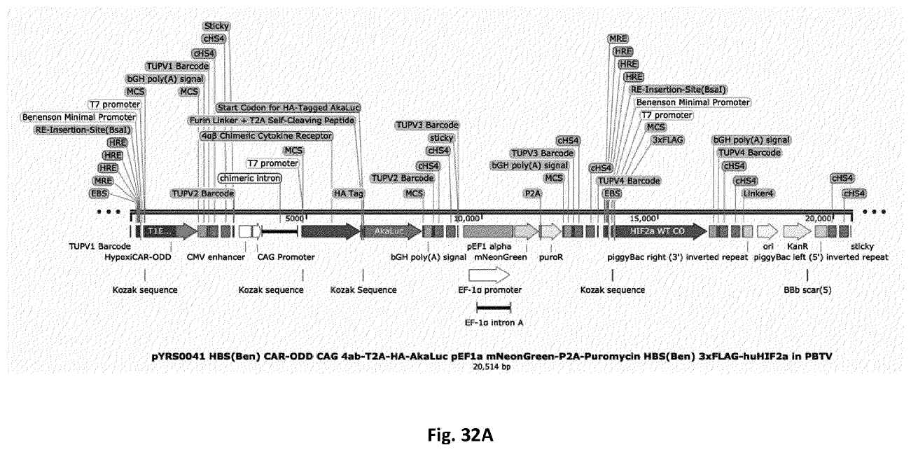

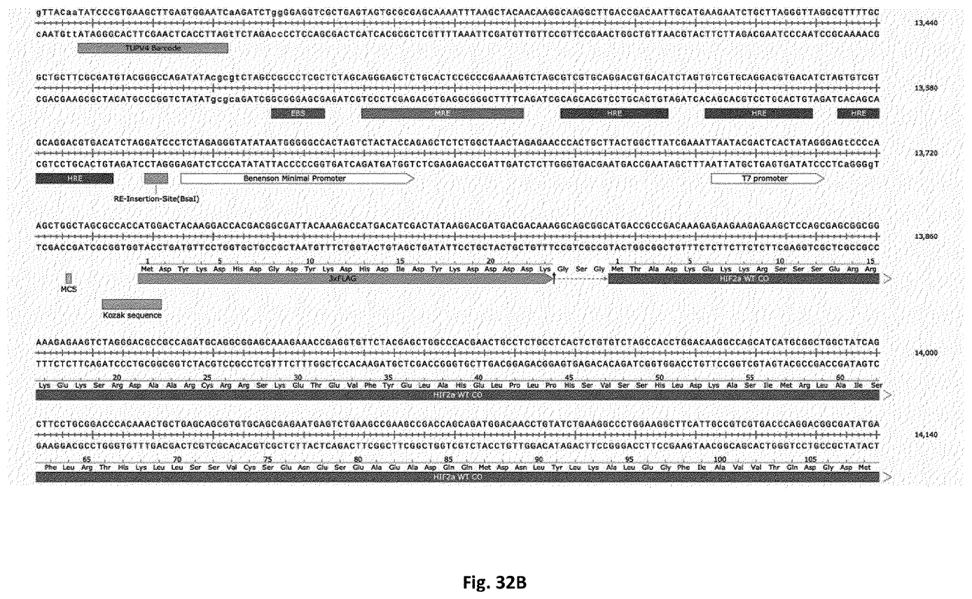

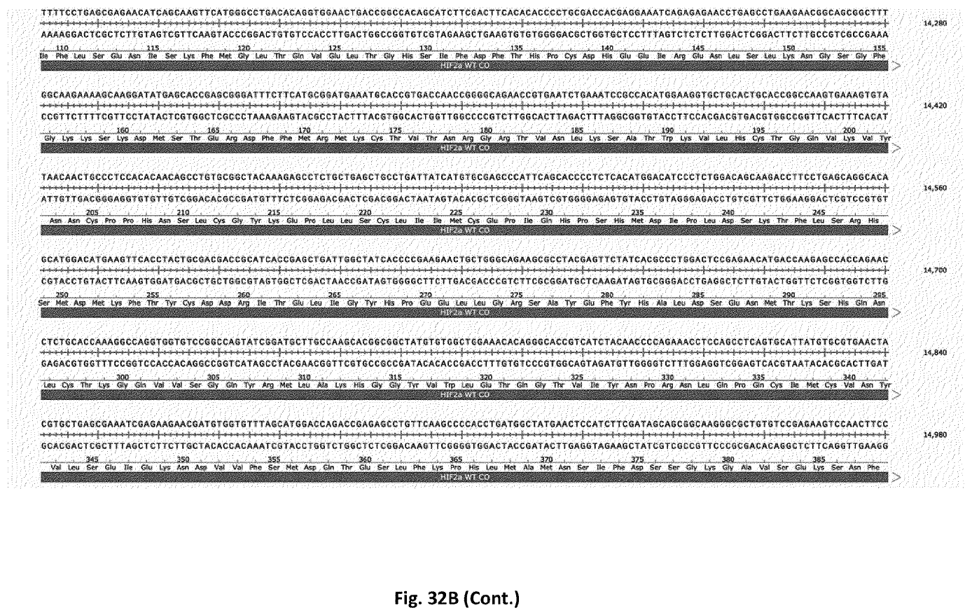

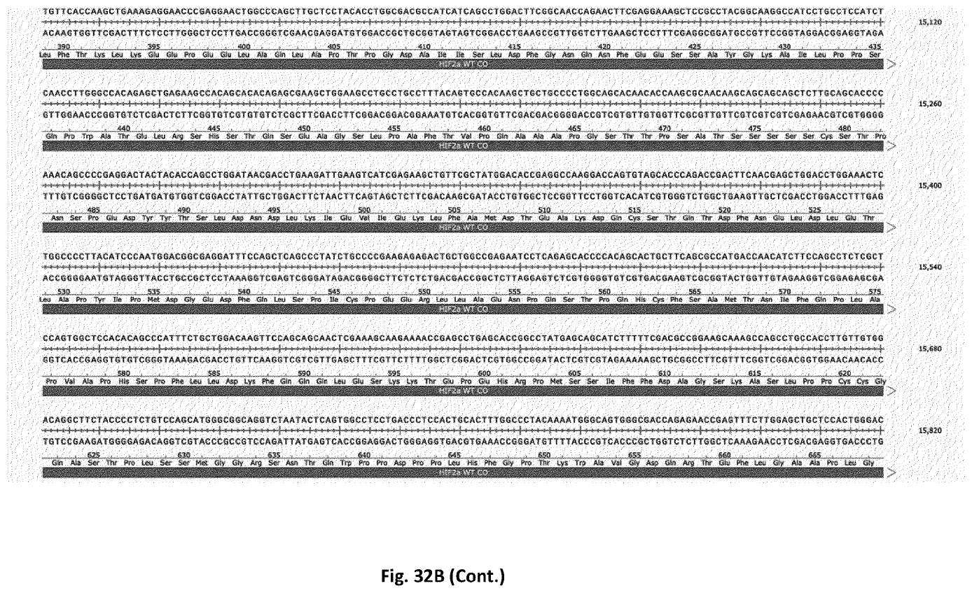

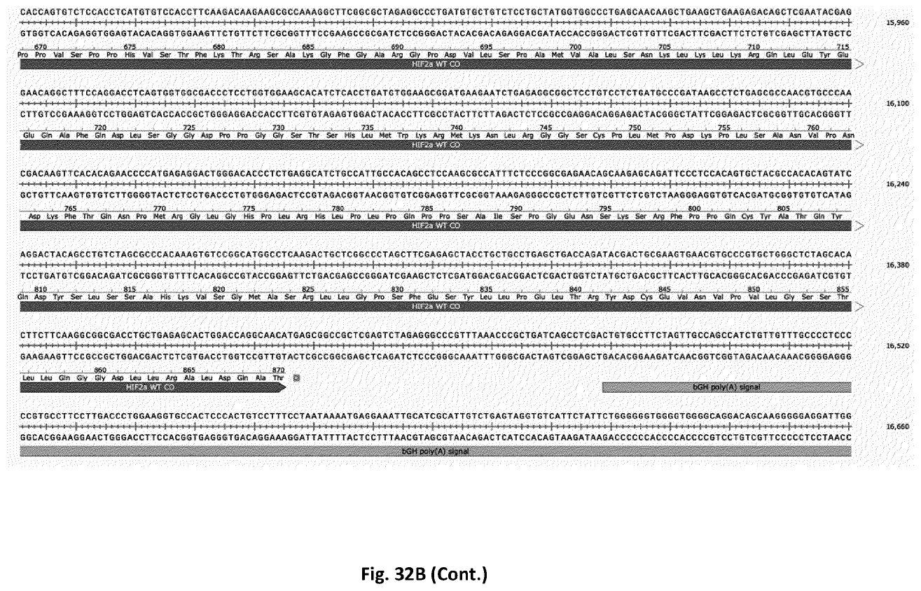

A series of circuits regulating the expression of a chimeric antigen receptor (CAR) demonstrate the utility of the hypoxia biosensors described herein. HEK293FT cell lines were generated to include various genomically integrated expression constructs (HBS-CAR-ODD (FIG. 22), HBS-CAR (FIG. 24), EF1α-CAR (FIG. 23), EF1α-CAR-ODD (FIG. 25), EF1α-Null (FIG. 26), HBS-huHIF1α-T2A-CAR-ODD (FIG. 27), HBS-CAR-ODD-T2A-huHIF1α (FIG. 28), HBS-huHIF2α-T2A-CAR-ODD (FIG. 29), HBS-CAR-ODD-T2A-huHIF2α (FIG. 30), multiple transcriptional unit HBS-CAR-ODD and HBS-huHIF1α (FIG. 31), and multiple transcriptional unit HBS-CAR-ODD and HBS-huHIF2α (FIG. 32)). Constructs that contain human HIF1α (huHIF1α) contain a codon-optimized huHIF1α nucleotide sequence set forth in SEQ ID NO: 7, which encodes a huHIF1α protein sequence set forth in SEQ ID NO: 8. Constructs that contain human HIF2α (huHIF2α) contain a codon-optimized huHIF2α nucleotide sequence set forth in SEQ ID NO: 9, which encodes a huHIF2α protein sequence set forth in SEQ ID NO: 10. To characterize the performance of each construct, cells were cultured for 4 d in either normoxia (21% O2) or hypoxia (1% O2) before CAR expression was analyzed by flow cytometry.

SEQ ID NO: 7
codon optimized human HIF1α (huHIF1α) DNA
ATGGAAGGCGCTGGCGGAGCCAACGACAAGAAGAAGATTAGCAGCGAGCGGCGGAAAGAGAAGTCCAGGGACGCTGC
CAGAAGCAGGCGGAGCAAAGAAAGCGAGGTGTTCTACGAGCTGGCCCACCAACTGCCTCTGCCTCACAATGTGTCTA
GCCACCTGGACAAGGCCAGCGTGATGAGACTGACCATCAGCTACCTGAGAGTGCGGAAGCTGCTGGATGCTGGCGAC
CTGGACATCGAGGACGATATGAAGGCCCAGATGAACTGCTTCTACCTGAAGGCCCTGGACGGCTTCGTGATGGTGCT
GACCGATGACGGCGACATGATATACATTAGCGACAACGTGAACAAGTATATGGGGCTGACCCAGTTCGAGCTGACCG
GCCACTCCGTGTTCGACTTCACACACCCTTGCGACCACGAAGAGATGAGAGAGATGCTGACCCACCGGAACGGCCTG
GTCAAGAAGGGCAAAGAGCAGAATACCCAGCGGTCATTCTTCCTGCGGATGAAATGCACCCTGACCAGCCGGGGCAG
AACCATGAATATCAAGAGCGCCACTTGGAAAGTGCTGCACTGCACCGGCCACATCCATGTGTATGACACCAACAGCA
ACCAGCCTCAGTGCGGCTACAAGAAACCTCCTATGACCTGCCTGGTGCTGATTTGCGAGCCCATTCCTCATCCTAGC
AATATCGAGATTCCCCTGGACAGCAAGACCTTCCTGAGCAGACACAGCCTGGACATGAAGTTTAGCTACTGCGACGA
GAGAATCACCGAGCTGATGGGCTACGAGCCCGAGGAACTTCTCGGACGGTCCATCTACGAGTATTATCACGCCCTGG
ACTCCGACCACCTGACCAAGACACACCACGATATGTTCACCAAGGGCCAAGTGACCACCGGCCAGTATAGAATGCTG
GCTAAGCGCGGAGGCTATGTGTGGGTTGAGACACAGGCCACCGTCATCTACAACACCAAGAACTCCCAGCCACAGTG
CATCGTGTGCGTGAACTACGTCGTGTCCGGCATCATCCAGCACGACCTGATTTTCAGCCTGCAACAGACCGAGTGCG
TGCTGAAGCCTGTGGAAAGCAGCGACATGAAGATGACCCAGCTTTTTACCAAGGTGGAATCCGAGGACACCAGCAGC
CTGTTCGACAAGCTGAAGAAAGAGCCCGACGCTCTGACACTGCTGGCTCCAGCAGCAGGCGATACCATCATCAGCCT
GGATTTCGGCAGCAACGACACCGAGACAGACGACCAGCAGCTCGAAGAGGTGCCCCTGTATAACGATGTGATGCTGC
CCTCTCCAAACGAGAAGCTCCAGAACATCAACCTGGCTATGAGCCCTCTGCCTACCGCCGAGACACCTAAGCCTCTG
AGAAGCTCTGCTGACCCCGCTCTGAATCAAGAAGTGGCCCTGAAGCTGGAACCCAATCCTGAGAGCCTGGAACTGAG
CTTCACCATGCCTCAGATTCAGGACCAGACACCTTCTCCAAGCGACGGCAGCACAAGACAGTCTAGCCCCGAGCCTA
ACAGCCCCAGCGAGTATTGCTTCTACGTGGACAGCGATATGGTCAATGAGTTCAAACTGGAACTGGTCGAGAAGCTG
TTCGCCGAAGATACCGAGGCCAAGAATCCCTTCAGCACCCAAGACACTGACCTGGACCTGGAAATGCTGGCCCCTTA
CATCCCTATGGACGACGACTTTCAGCTCCGGTCCTTCGACCAGCTCAGCCCTCTGGAATCCAGCTCTGCCTCTCCAG
AAAGCGCCTCTCCACAGAGCACCGTGACCGTGTTTCAGCAGACCCAGATTCAAGAGCCCACCGCCAATGCCACCACC
ACAACCGCCACAACAGACGAGCTGAAAACCGTGACCAAGGACCGCATGGAGGACATCAAGATTCTGATTGCCTCTCC
TTCTCCGACGCACATCCACAAAGAGACAACCAGCGCCACAAGCAGCCCCTACAGAGACACCCAGAGCAGAACCGCCT
CTCCTAATAGAGCCGGAAAGGGCGTCATCGAGCAGACCGAGAAGTCTCACCCTAGAAGCCCCAACGTGCTGAGCGTG
GCACTGAGCCAGAGAACCACAGTGCCCGAAGAGGAACTGAACCCTAAGATTCTGGCCCTCCAGAACGCCCAGCGGAA
GCGGAAAATGGAACACGACGGAAGCCTGTTCCAGGCCGTCGGAATCGGAACACTGCTGCAACAGCCTGATGACCACG
CCGCCACCACAAGCCTGTCTTGGAAGAGAGTGAAGGGCTGCAAGTCCAGCGAGCAGAACGGAATGGAACAGAAAACC
ATCATCCTGATTCCTAGCGACCTGGCCTGTAGACTGCTGGGCCAGAGCATGGATGAGAGCGGACTGCCACAGCTCAC
CAGCTACGACTGCGAAGTGAACGCCCCTATCCAGGGCAGCAGAAACCTGCTCCAAGGCGAGGAACTGCTGCGAGCAC
TGGACCAAGTGAACTGA
SEQ ID NO: 8
codon optimized human HIF1α (huHIF1α) protein
MEGAGGANDKKKISSERRKEKSRDAARSRRSKESEVFYELAHQLPLPHNVSSHLDKASVMRLTISYLRVRKLLDAGD
LDIEDDMKAQMNCFYLKALDGFVMVLTDDGDMIYISDNVNKYMGLTQFELTGHSVFDFTHPCDHEEMREMLTHRNGL
VKKGKEQNTQRSFFLRMKCTLTSRGRTMNIKSATWKVLHCTGHIHVYDTNSNQPQCGYKKPPMTCLVLICEPIPHPS
NIEIPLDSKTFLSRHSLDMKFSYCDERITELMGYEPEELLGRSIYEYYHALDSDHLTKTHHDMFTKGQVTTGQYRML
AKRGGYVWVETQATVIYNTKNSQPQCIVCVNYVVSGIIQHDLIFSLQQTECVLKPVESSDMKMTQLFTKVESEDTSS
LFDKLKKEPDALTLLAPAAGDTIISLDFGSNDTETDDQQLEEVPLYNDVMLPSPNEKLQNINLAMSPLPTAETPKPL
RSSADPALNQEVALKLEPNPESLELSFTMPQIQDQTPSPSDGSTRQSSPEPNSPSEYCFYVDSDMVNEFKLELVEKL
FAEDTEAKNPFSTQDTDLDLEMLAPYIPMDDDFQLRSFDQLSPLESSSASPESASPQSTVTVFQQTQIQEPTANATT
TTATTDELKTVTKDRMEDIKILIASPSPTHIHKETTSATSSPYRDTQSRTASPNRAGKGVIEQTEKSHPRSPNVLSV
ALSQRTTVPEEELNPKILALQNAQRKRKMEHDGSLFQAVGIGTLLQQPDDHAATTSLSWKRVKGCKSSEQNGMEQKT
IILIPSDLACRLLGQSMDESGLPQLTSYDCEVNAPIQGSRNLLQGEELLRALDQVN*
SEQ ID NO: 9
codon optimized human HIF2α (huHIF2α) DNA
ATGACCGCCGACAAAGAGAAGAAGAGAAGCTCCAGCGAGCGGCGGAAAGAGAAGTCTAGGGACGCCGCCAGATGCAG
GCGGAGCAAAGAAACCGAGGTGTTCTACGAGCTGGCCCACGAACTGCCTCTGCCTCACTCTGTGTCTAGCCACCTGG
ACAAGGCCAGCATCATGCGGCTGGCTATCAGCTTCCTGCGGACCCACAAACTGCTGAGCAGCGTGTGCAGCGAGAAT
GAGTCTGAAGCCGAAGCCGACCAGCAGATGGACAACCTGTATCTGAAGGCCCTGGAAGGCTTCATTGCCGTCGTGAC
CCAGGACGGCGATATGATTTTCCTGAGCGAGAACATCAGCAAGTTCATGGGCCTGACACAGGTGGAACTGACCGGCC
ACAGCATCTTCGACTTCACACACCCCTGCGACCACGAGGAAATCAGAGAGAACCTGAGCCTGAAGAACGGCAGCGGC
TTTGGCAAGAAAAGCAAGGATATGAGCACCGAGCGGGATTTCTTCATGCGGATGAAATGCACCGTGACCAACCGGGG
CAGAACCGTGAATCTGAAATCCGCCACATGGAAGGTGCTGCACTGCACCGGCCAAGTGAAAGTGTATAACAACTGCC
CTCCACACAACAGCCTGTGCGGCTACAAAGAGCCTCTGCTGAGCTGCCTGATTATCATGTGCGAGCCCATTCAGCAC
CCCTCTCACATGGACATCCCTCTGGACAGCAAGACCTTCCTGAGCAGGCACAGCATGGACATGAAGTTCACCTACTG
CGACGACCGCATCACCGAGCTGATTGGCTATCACCCCGAAGAACTGCTGGGCAGAAGCGCCTACGAGTTCTATCACG
CCCTGGACTCCGAGAACATGACCAAGAGCCACCAGAACCTCTGCACCAAAGGCCAGGTGGTGTCCGGCCAGTATCGG
ATGCTTGCCAAGCACGGCGGCTATGTGTGGCTGGAAACACAGGGCACCGTCATCTACAACCCCAGAAACCTCCAGCC
TCAGTGCATTATGTGCGTGAACTACGTGCTGAGCGAAATCGAGAAGAACGATGTGGTGTTTAGCATGGACCAGACCG
AGAGCCTGTTCAAGCCCCACCTGATGGCTATGAACTCCATCTTCGATAGCAGCGGCAAGGGCGCTGTGTCCGAGAAG
TCCAACTTCCTGTTCACCAAGCTGAAAGAGGAACCCGAGGAACTGGCCCAGCTTGCTCCTACACCTGGCGACGCCAT
CATCAGCCTGGACTTCGGCAACCAGAACTTCGAGGAAAGCTCCGCCTACGGCAAGGCCATCCTGCCTCCATCTCAAC
CTTGGGCCACAGAGCTGAGAAGCCACAGCACACAGAGCGAAGCTGGAAGCCTGCCTGCCTTTACAGTGCCACAAGCT
GCTGCCCCTGGCAGCACAACACCAAGCGCAACAAGCAGCAGCAGCTCTTGCAGCACCCCAAACAGCCCCGAGGACTA
CTACACCAGCCTGGATAACGACCTGAAGATTGAAGTCATCGAGAAGCTGTTCGCTATGGACACCGAGGCCAAGGACC
AGTGTAGCACCCAGACCGACTTCAACGAGCTGGACCTGGAAACTCTGGCCCCTTACATCCCAATGGACGGCGAGGAT
TTCCAGCTCAGCCCTATCTGCCCCGAAGAGAGACTGCTGGCCGAGAATCCTCAGAGCACCCCACAGCACTGCTTCAG
CGCCATGACCAACATCTTCCAGCCTCTCGCTCCAGTGGCTCCACACAGCCCATTTCTGCTGGACAAGTTCCAGCAGC
AACTCGAAAGCAAGAAAACCGAGCCTGAGCACCGGCCTATGAGCAGCATCTTTTTCGACGCCGGAAGCAAAGCCAGC
CTGCCACCTTGTTGTGGACAGGCTTCTACCCCTCTGTCCAGCATGGGCGGCAGGTCTAATACTCAGTGGCCTCCTGA
CCCTCCACTGCACTTTGGCCCTACAAAATGGGCAGTGGGCGACCAGAGAACCGAGTTTCTTGGAGCTGCTCCACTGG
GACCACCAGTGTCTCCACCTCATGTGTCCACCTTCAAGACAAGAAGCGCCAAAGGCTTCGGCGCTAGAGGCCCTGAT
GTGCTGTCTCCTGCTATGGTGGCCCTGAGCAACAAGCTGAAGCTGAAGAGACAGCTCGAATACGAGGAACAGGCTTT
CCAGGACCTCAGTGGTGGCGACCCTCCTGGTGGAAGCACATCTCACCTGATGTGGAAGCGGATGAAGAATCTGAGAG
GCGGCTCCTGTCCTCTGATGCCCGATAAGCCTCTGAGCGCCAACGTGCCCAACGACAAGTTCACACAGAACCCCATG
AGAGGACTGGGACACCCTCTGAGGCATCTGCCATTGCCACAGCCTCCAAGCGCCATTTCTCCCGGCGAGAACAGCAA
GAGCAGATTCCCTCCACAGTGCTACGCCACACAGTATCAGGACTACAGCCTGTCTAGCGCCCACAAAGTGTCCGGCA
TGGCCTCAAGACTGCTCGGCCCTAGCTTCGAGAGCTACCTGCTGCCTGAGCTGACCAGATACGACTGCGAAGTGAAC
GTGCCCGTGCTGGGCTCTAGCACACTTCTTCAAGGCGGCGACCTGCTGAGAGCACTGGACCAGGCAACATGA
SEQ ID NO: 10
codon optimized human HIF2α (huHIF2α) Protein
MTADKEKKRSSSERRKEKSRDAARCRRSKETEVFYELAHELPLPHSVSSHLDKASIMRLAISFLRTHKLLSSVCSEN
ESEAEADQQMDNLYLKALEGFIAVVTQDGDMIFLSENISKFMGLTQVELTGHSIFDFTHPCDHEEIRENLSLKNGSG
FGKKSKDMSTERDFFMRMKCTVTNRGRTVNLKSATWKVLHCTGQVKVYNNCPPHNSLCGYKEPLLSCLIIMCEPIQH
PSHMDIPLDSKTFLSRHSMDMKFTYCDDRITELIGYHPEELLGRSAYEFYHALDSENMTKSHQNLCTKGQVVSGQYR
MLAKHGGYVWLETQGTVIYNPRNLQPQCIMCVNYVLSEIEKNDVVFSMDQTESLFKPHLMAMNSIFDSSGKGAVSEK
SNFLFTKLKEEPEELAQLAPTPGDAIISLDFGNQNFEESSAYGKAILPPSQPWATELRSHSTQSEAGSLPAFTVPQA
AAPGSTTPSATSSSSSCSTPNSPEDYYTSLDNDLKIEVIEKLFAMDTEAKDQCSTQTDFNELDLETLAPYIPMDGED
FQLSPICPEERLLAENPQSTPQHCFSAMTNIFQPLAPVAPHSPELLDKFQQQLESKKTEPEHRPMSSIFFDAGSKAS
LPPCCGQASTPLSSMGGRSNTQWPPDPPLHFGPTKWAVGDQRTEFLGAAPLGPPVSPPHVSTEKTRSAKGFGARGPD
VLSPAMVALSNKLKLKRQLEYEEQAFQDLSGGDPPGGSTSHLMWKRMKNLRGGSCPLMPDKPLSANVPNDKFTQNPM
RGLGHPLRHLPLPQPPSAISPGENSKSRFPPQCYATQYQDYSLSSAHKVSGMASRLLGPSFESYLLPELTRYDCEVN
VPVLGSSTLLQGGDLLRALDQAT*

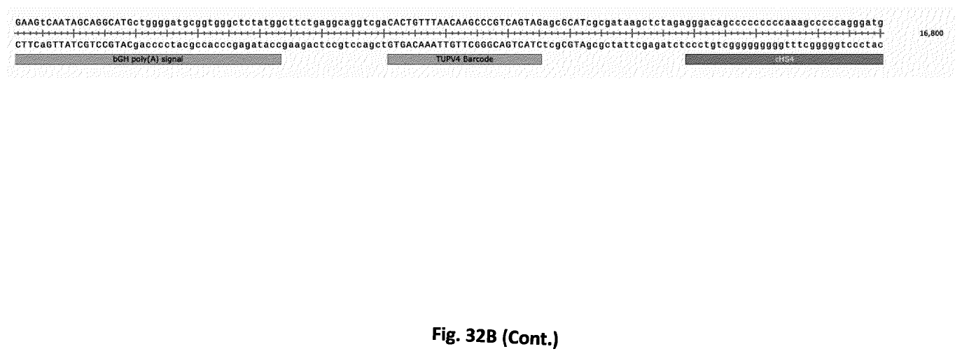

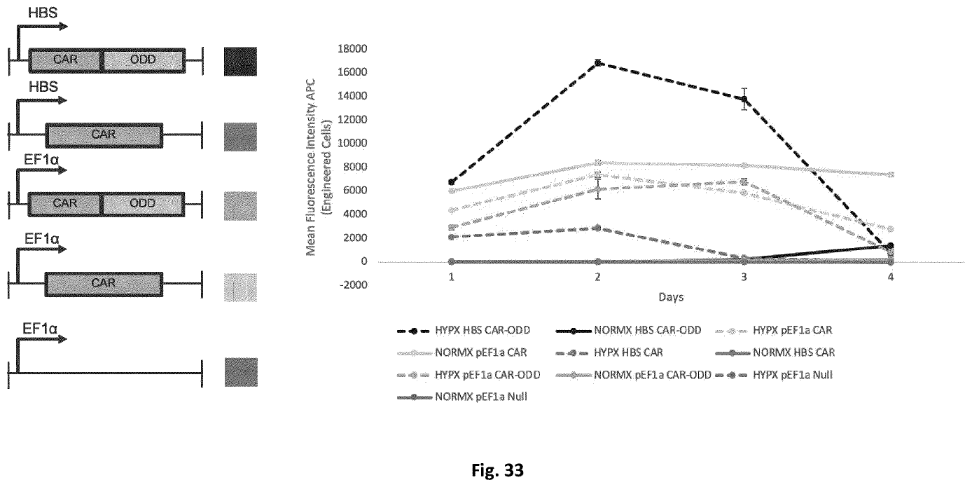

The first series of open-loop (no feedback) constructs was used for benchmarking (FIG. 33). Of these constructs, the highest CAR expression (as measured by mean APC fluorescence intensity) was achieved by the HBS-CAR-ODD construct cultured under hypoxic conditions. The second highest level of CAR expression was achieved by the EF1α-CAR construct cultured under normoxic conditions. The EF1α-CAR construct cultured in hypoxia exhibited similar levels of CAR expression compared to the EF1α-CAR-ODD cultured in hypoxia. Low amounts of CAR expression were initially observed from the HBS-CAR construct under hypoxic culture, but these expression levels decreased to similar levels as the null construct containing no CAR after 3 d in culture. With the exception of the EF1α-CAR construct, all constructs in cells cultured at normoxia exhibit minimal or no expression of CAR, as benchmarked to the EF1α-Null construct containing no CAR protein.

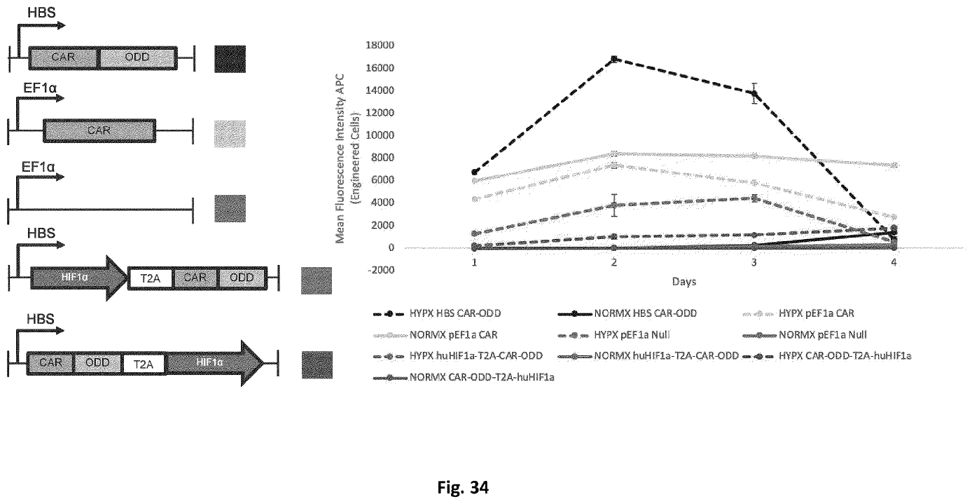

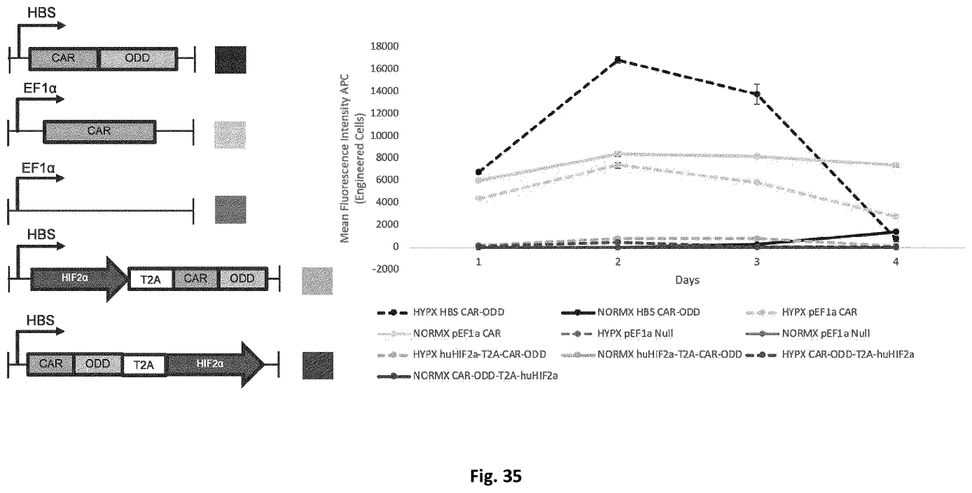

The next series of constructs included feedback in which a hypoxia-inducible factor was expressed co-cistronically with the CAR gene (FIG. 34, 35). For constructs based upon human HIF1α (huHIF1α) (FIG. 34), hypoxia-inducible CAR expression was observed for both biosensor configurations. However, the magnitude of CAR expression was lower than that observed using the open loop controls. Constructs based upon human HIF2α (huHIF2α) (FIG. 35), also exhibited hypoxia-inducible expression but the magnitude was lower than either open loop controls or circuits based upon huHIF1α. In both co-cistronic huHIF1α and huHIF2α circuits, placement of the huHIF1α or huHIF2α prior to the intervening self-cleaving T2A peptide produced greater levels of CAR expression from the circuit when compared to placement of the huHIF1α or huHIF2α after the T2A peptide. All co-cistronic huHIF1α and huHIF2α feedback constructs in cells cultured at normoxia exhibited minimal or no expression of CAR, indicating the desired minimal background expression. These findings indicate that both the composition and the configuration of the biosensor circuit impact the magnitude of output gene expression conferred by exposure to hypoxia.

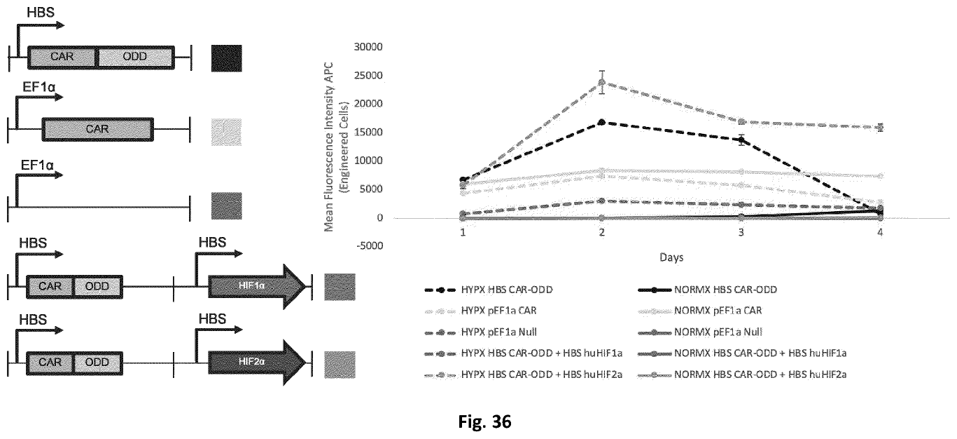

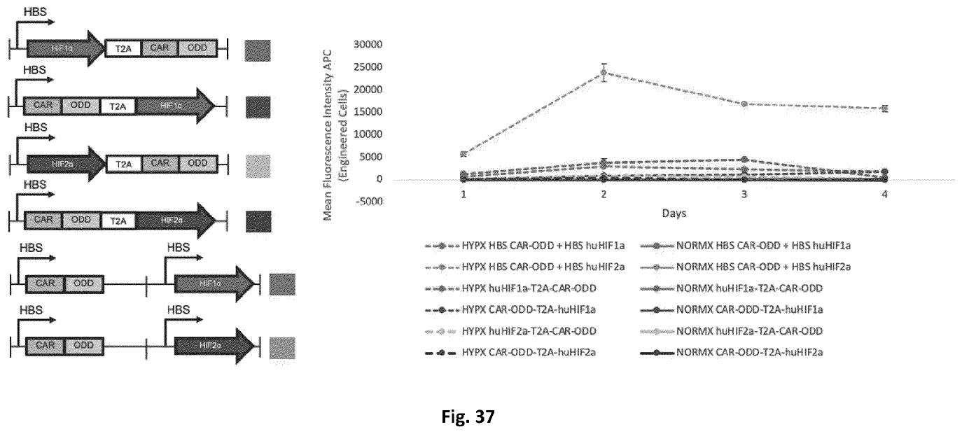

The next series of constructs implemented a similar feedback topology, but with the CAR and hypoxia-inducible factors expressed from separate transcriptional units (FIG. 36, 37). This comparison was motivated by the fact that co-cistronic expression using self-cleaving peptides can occasionally yield fusion proteins due to translational read-thorough. When implemented in multiple transcriptional units, the huHIF1α feedback circuit conferred hypoxia-inducible expression (FIG. 36), and this circuit produced levels of CAR expression similar to those achieved with co-cistronic feedback using huHIF1α (see comparison in FIG. 37). The multiple transcription unit huHIF2α feedback circuit also conferred hypoxia-inducible expression (FIG. 36), and strikingly, this circuit produced levels of hypoxia-inducible CAR expression that were greater in magnitude than all other cases evaluated including both open loop strategies and other feedback circuit designs (see comparison in FIG. 37). All multiple transcriptional unit huHIF1α and huHIF2α feedback constructs again conferred minimal background CAR expression in normoxia, as desired. Altogether, these data indicated that genetic circuit designs exhibiting the same feedback topology can confer substantially different performance characteristics as a function of circuit design.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

### REFERENCES

1. Whiteside T L. The tumor microenvironment and its role in promoting tumor growth. Oncogene 27, 5904-5912 (2008).
2. Hockel M, Vaupel P. Tumor hypoxia: definitions and current clinical, biologic, and molecular aspects. J Natl Cancer Inst 93, 266-276 (2001).
3. Vaupel P, Kelleher D K, Hockel M. Oxygen status of malignant tumors: pathogenesis of hypoxia and significance for tumor therapy. Semin Oncol 28, 29-35 (2001).
4. Vaupel P, Okunieff P, Neuringer L J. Blood flow, tissue oxygenation, pH distribution, and energy metabolism of murine mammary adenocarcinomas during growth. Adv Exp Med Biol 248, 835-845 (1989).
5. Siemann D W. The unique characteristics of tumor vasculature and preclinical evidence for its selective disruption by Tumor-Vascular Disrupting Agents. Cancer Treat Rev 37, 63-74 (2011).
6. Michiels C. Physiological and pathological responses to hypoxia. Am J Pathol 164, 1875-1882 (2004).
7. Lee J Y, et al. A novel chimeric promoter that is highly responsive to hypoxia and metals. Gene Ther 13, 857-868 (2006).
8. Iglesias P, Costoya J A. A novel BRET-based genetically encoded biosensor for functional imaging of hypoxia. Biosens Bioelectron 24, 3126-3130 (2009).
9. Iglesias P, Fraga M, Costoya J A. Defining hypoxic microenvironments by non-invasive functional optical imaging. Eur J Cancer 49, 264-271 (2013).
10. Ede C, Chen X, Lin M Y, Chen Y Y. Quantitative Analyses of Core Promoters Enable Precise Engineering of Regulated Gene Expression in Mammalian Cells. ACS Synth Biol 5, 395-404 (2016).
11. Koh M Y, Powis G. Passing the baton: the HIF switch. Trends Biochem Sci 37, 364-372 (2012).
12. Uchida T, et al. Prolonged hypoxia differentially regulates hypoxia-inducible factor (HIF)-1alpha and HIF-2alpha expression in lung epithelial cells: implication of natural antisense HIF-1alpha. J Biol Chem 279, 14871-14878 (2004).
13. Gordan J D, Simon M C. Hypoxia-inducible factors: central regulators of the tumor phenotype. Curr Opin Genet Dev 17, 71-77 (2007).
14. Holmquist-Mengelbier L, et al. Recruitment of HIF-1alpha and HIF-2alpha to common target genes is differentially regulated in neuroblastoma: HIF-2alpha promotes an aggressive phenotype. Cancer Cell 10, 413-423 (2006).
15. Jochmanová I, Zelinka T, Widimský J, Jr., Pacak K. HIF signaling pathway in pheochromocytoma and other neuroendocrine tumors. Physiol Res 63, S251-262 (2014).
16. Löfstedt T, et al. Hypoxia inducible factor-2alpha in cancer. Cell Cycle 6, 919-926 (2007).
17. Semenza G L. HIF-1 mediates the Warburg effect in clear cell renal carcinoma. J Bioenerg Biomembr 39, 231-234 (2007).
18. Tiburcio P D, Choi H, Huang L E. Complex role of HIF in cancer: the known, the unknown, and the unexpected. Hypoxia (Auckl) 2, 59-70 (2014).
19. Jun J C, Rathore A, Younas H, Gilkes D, Polotsky V Y. Hypoxia-Inducible Factors and Cancer. Curr Sleep Med Rep 3, 1-10 (2017).
20. Iommarini L, Porcelli A M, Gasparre G, Kurelac I. Non-Canonical Mechanisms Regulating Hypoxia-Inducible Factor 1 Alpha in Cancer. Front Oncol 7, 286 (2017).
21. Donahue P S, Draut J W, Muldoon J J, Edelstein H I, Bagheri N, Leonard J N. The COMET toolkit for composing customizable genetic programs in mammalian cells. Nat Commun 11, 779 (2020).
22. Muldoon J J, et al. Model-guided design of mammalian genetic programs. bioRxiv, 2020.2009.2030.320853 (2020).
23. Duportet X, et al. A platform for rapid prototyping of synthetic gene networks in mammalian cells. Nucleic Acids Res 42, 13440-13451 (2014).
24. Gaidukov L, et al. A multi-landing pad DNA integration platform for mammalian cell engineering. Nucleic Acids Res 46, 4072-4086 (2018).
25. Fitzgerald M, Livingston M, Gibbs C, Deans T L. Rosa26 docking sites for investigating genetic circuit silencing in stem cells. Synthetic Biology, (2020).
26. Place T L, Domann F E, Case A J. Limitations of oxygen delivery to cells in culture: An underappreciated problem in basic and translational research. Free Radic Biol Med 113, 311-322 (2017).
27. Wenger R H, Kurtcuoglu V, Scholz C C, Marti H H, Hoogewijs D. Frequently asked questions in hypoxia research. Hypoxia (Auckl) 3, 35-43 (2015).
28. Erapaneedi R, Belousov V V, Schäfers M, Kiefer F. A novel family of fluorescent hypoxia sensors reveal strong heterogeneity in tumor hypoxia at the cellular level. Embo j 35, 102-113 (2016).
29. Misra T, et al. A genetically encoded biosensor for visualising hypoxia responses in vivo. Biol Open 6, 296-304 (2017).
30. Yuan Y, Hilliard G, Ferguson T, Millhorn D E. Cobalt inhibits the interaction between hypoxia-inducible factor-alpha and von Hippel-Lindau protein by direct binding to hypoxia-inducible factor-alpha. J Biol Chem 278, 15911-15916 (2003).
31. Hu C J, Sataur A, Wang L, Chen H, Simon M C. The N-terminal transactivation domain confers target gene specificity of hypoxia-inducible factors HIF-1alpha and HIF-2alpha. Mol Biol Cell 18, 4528-4542 (2007).
32. Iyer N V, Leung S W, Semenza G L. The human hypoxia-inducible factor 1alpha gene: HIF1A structure and evolutionary conservation. Genomics 52, 159-165 (1998).
33. Hon W C, et al. Structural basis for the recognition of hydroxyproline in HIF-1 alpha by pVHL. Nature 417, 975-978 (2002).
34. Iglesias P, Penas C, Barral-Cagiao L, Pazos E, Costoya J A. A Bio-inspired Hypoxia Sensor using HIF1α-Oxygen-Dependent Degradation Domain. Sci Rep 9, 7117 (2019).
35. Konermann S, et al. Genome-scale transcriptional activation by an engineered CRISPR-Cas9 complex. Nature 517, 583-588 (2015).
36. Bintu L, et al. Dynamics of epigenetic regulation at the single-cell level. Science 351, 720-724 (2016).
37. Ai H W, Shaner N C, Cheng Z, Tsien R Y, Campbell R E. Exploration of new chromophore structures leads to the identification of improved blue fluorescent proteins. Biochemistry 46, 5904-5910 (2007).
38. Daringer N M, Dudek R M, Schwarz K A, Leonard J N. Modular extracellular sensor architecture for engineering mammalian cell-based devices. ACS Synth Biol 3, 892-902 (2014).
39. Subach O M, Cranfill P J, Davidson M W, Verkhusha V V. An enhanced monomeric blue fluorescent protein with the high chemical stability of the chromophore. PLOS One 6, e28674 (2011).
40. Shaner N C, et al. A bright monomeric green fluorescent protein derived from Branchiostoma lanceolatum. Nat Methods 10, 407-409 (2013).
41. Bajar B T, et al. Improving brightness and photostability of green and red fluorescent proteins for live cell imaging and FRET reporting. Sci Rep 6, 20889 (2016).
42. Shemetov A A, Oliinyk O S, Verkhusha V V. How to Increase Brightness of Near-Infrared Fluorescent Proteins in Mammalian Cells. Cell Chem Biol 24, 758-766.e753 (2017).
43. Oliinyk O S, Shemetov A A, Pletnev S, Shcherbakova D M, Verkhusha V V. Smallest near-infrared fluorescent protein evolved from cyanobacteriochrome as versatile tag for spectral multiplexing. Nat Commun 10, 279 (2019).
44. Shcherbakova D M, Cox Cammer N, Huisman T M, Verkhusha V V, Hodgson L. Direct multiplex imaging and optogenetics of Rho GTPases enabled by near-infrared FRET. Nat Chem Biol 14, 591-600 (2018).
45. Chu V T, et al. Increasing the efficiency of homology-directed repair for CRISPR-Cas9-induced precise gene editing in mammalian cells. Nat Biotechnol 33, 543-548 (2015).
46. Chu V T, et al. Efficient generation of Rosa26 knock-in mice using CRISPR/Cas9 in C57BL/6 zygotes. BMC Biotechnol 16, 4 (2016).

1. A DNA-based hypoxia biosensor, comprising:
(a) a hypoxia-inducible promoter;
(b) a nucleic acid sequence encoding a functional element; and
(c) at least one nucleic acid sequence encoding a feedback element.

2. The hypoxia biosensor of claim 1, wherein the hypoxia-inducible promoter comprises (i) an Egr-1-binding site (EBS) from a Egr-1 gene, a metal-response element (MRE) from a metallothionein gene, a hypoxia-response element (HRE), or a combination thereof, and (ii) a minimal promoter.

3. The hypoxia biosensor of claim 1, wherein the hypoxia-inducible promoter comprises (i) an Egr-1-binding site (EBS) from a Egr-1 gene, a metal-response element (MRE) from a metallothionein gene, and at least one a hypoxia-response element (HRE), and (ii) a minimal promoter.

4. (canceled)

5. The hypoxia biosensor of claim 1, wherein the minimal promoter is selected from simian virus 40 (SV40) promoter, cytomegalovirus (CMV) promoter, and a synthetic promoter.

6. (canceled)

7. The hypoxia biosensor of claim 1, wherein the functional element is (i) a gene sequence or fragment thereof, a nucleic acid sequence encoding a regulatory RNA molecule, or a reporter element, or (ii) a reporter element.

8-11. (canceled)

12. The hypoxia biosensor of claim 1, wherein the feedback element is a positive feedback element.

13. The hypoxia biosensor of claim 12, wherein the positive feedback element is (i) selected from HIF1α and HIF2α, (ii) either in-whole or in-part a gene that occurs in nature, or (iii) either in-whole or in-part a gene that is of synthetic origin.

14-16. (canceled)

17. The hypoxia biosensor of claim 1, wherein the hypoxia biosensor comprises a nucleic acid sequence encoding a first positive feedback element, and a nucleic acid sequence encoding a second positive feedback element.

18. (canceled)

19. The hypoxia biosensor of claim 1, wherein the functional element (i) comprises at least one oxygen-degradation domain (ODD), or (ii) is a chimeric antigen receptor (CAR).

20. (canceled)

21. The hypoxia biosensor of claim 1, wherein the nucleic acid sequence encoding a functional element and the at least one nucleic acid sequence encoding a feedback element are separated by a nucleic acid sequence encoding a cleavage peptide.

22-24. (canceled)

25. The hypoxia biosensor of claim 1, wherein the hypoxia biosensor comprises at least two hypoxia-inducible promoters.

26-28. (canceled)

29. A DNA-based hypoxia biosensor, comprising:
(a) a hypoxia-inducible promoter;
(b) a nucleic acid sequence encoding a functional element;
(c) at least one nucleic acid sequence encoding one or more engineered proteins selected from the group consisting of:
(i) an engineered protein that activates gene expression, wherein the engineered protein comprises a DNA binding domain and a transcription activator domain;
(ii) an engineered protein that inhibits gene expression;
(iii) an engineered protein that inhibits gene expression, the engineered protein optionally comprising one or more of a DNA binding domain, a bulky domain, a chromatin remodeling domain, and a transcription inhibitor domain; and
(iv) a combination of two engineered proteins comprising a first engineered protein comprising a DNA binding domain fused to a dimerization domain, and a second engineered protein comprising a transcription regulator domain fused to a dimerization domain, wherein the dimerization domains of the two engineered proteins dimerize in the presence of a stimulus to which the dimerization domains of the two engineered proteins bind; and
(d) one or more DNA binding sites for the DNA binding domain of the engineered protein(s) of (c).

30. The hypoxia biosensor of claim 29, wherein the functional element is a reporter element.

31-33. (canceled)

34. The hypoxia biosensor of claim 29, wherein the engineered protein comprises a positive feedback element.

35. The hypoxia biosensor of claim 29, wherein the hypoxia-inducible promoter comprises a minimal promoter selected from the group consisting of simian virus 40 (SV40) promoter, cytomegalovirus (CMV) promoter, and a synthetic promoter.

36. (canceled)

37. The hypoxia biosensor of claim 29, wherein the engineered protein (i) is a COMET transcription factor (COMET TF), or (ii) comprises at least one oxygen-degradation domain (ODD).

38. (canceled)

39. The hypoxia biosensor of claim 29, wherein at least one DNA binding site for the DNA binding domain of the engineered protein(s) is:
(a) 5′ of the nucleic acid sequence encoding the functional element;
(b) 5′ of the hypoxia-inducible promoter; or
(c) 5′ of at least one nucleic acid sequence encoding the one or more engineered proteins.

40. (canceled)

41. The hypoxia biosensor of claim 29, wherein at least one DNA binding site for the DNA binding domain of the engineered protein(s) is:
(a) 5′ of the hypoxia-inducible promoter;
(b) 5′ of at least one nucleic acid sequence encoding the one or more engineered proteins; or
(c) 5′ of the nucleic acid sequence encoding the functional element.

42-44. (canceled)

45. The hypoxia biosensor of claim 29, wherein the biosensor comprises at least two nucleic acid sequences encoding one or more engineered proteins.

46. The hypoxia biosensor of claim 29, wherein the engineered protein(s) comprises at least one split intein on the C-terminus or N-terminus of the DNA binding domain, a transcription activator domain, or a combination thereof.


SEQUENCE LISTING (49 sequences)

SEQ ID NO: 1 - DNA, 2511 bp, Mus musculus
  source 1..2511: mol_type = other DNA
atggaaggtgctggcggcgagaacgagaagaaaaagatgtccagcgagcgccggaaagagaagtctagggacgccgccagaagcagaagatccaaagaaagcgaggtgttctacgagctggcccaccaactgcctctgcctcacaatgtgtctagccacctggacaaggccagcgtgatgagactgaccatcagctacctgagagtgcggaagctgctggatgctggcggactggatagcgaggatgagatgaaggcccagatggactgcttctacctgaaggccctggacggcttcgtgatggtgctgacagacgatggcgacatggtgtatatcagcgacaacgtgaacaagtacatggggctgacccagttcgagctgacaggccacagcgtgttcgacttcacacacccctgtgaccacgaagagatgagagagatgctgacccacagaaacggccccgtgcggaagggaaaagagctgaatacccagcggtcattcttcctgaggatgaagtgtaccctgaccagcaggggcagaaccatgaacatcaagagcgctacctggaaagtgctgcactgcaccggacacatccacgtgtacgacaccaacagcaaccagcctcagtgcggctacaagaaacctcctatgacctgcctggtgctgatctgcgagcccattcctcatcctagcaacatcgagatccctctggacagcaagaccttcctgagcagacacagcctggacatgaagttcagctactgcgacgagagaatcaccgagctgatgggctacgagcccgaggaacttctcggcagatccatctatgagtattaccacgctctggactccgaccacctgaccaagacacaccacgatatgttcaccaagggccaagtgaccaccggccagtacagaatgctggctaagagaggcggctacgtctgggtcgagacacaggctaccgtgatctacaacaccaagaactcccagccacagtgcatcgtgtgcgtgaactacgtcgtgtccgggatcatccagcacgacctgatctttagcctgcaacagaccgagagcgtgctgaagcctgtggaaagcagcgacatgaagatgacccagctgttcacaaaggtggaatccgaggacaccagctgcctgttcgacaagctgaagaaagagcccgacgctctgacactgctggctccagctgctggcgacacaatcatcagcctggatttcggcagcgacgacaccgagacagaggatcagcagctcgaagatgtgcccctgtataacgacgtgatgttccccagcagcaacgagaagctgaatatcaacctggccatgtctcctctgcctagctccgagacacccaagcctctgagaagctctgccgatcctgctctgaatcaagaggtggccctgaagctggaaagctcccctgaatctctgggcctgagcttcaccatgcctcagatccaggaccagcctgcctctccttctgacggctctacaagacagagcagccccgagagactgctgcaagagaatgtgaacaccccaaacttctcccagcctaacagccccagcgagtattgcttcgacgtggacagcgatatggtcaacgtgttcaagctggaactggtggaaaagctgttcgccgaggataccgaggctaagaaccccttcagcacccaagacaccgacctggaccttgagatgctggccccttacatccctatggacgacgacttccagctgagatccttcgaccagctgagcccactggaaagcaacagcccatctccacctagcatgagcaccgtgaccggctttcagcagactcagctccagaagcctaccatcaccgccaccgccacaacaacagccaccacagacgagagcaagaccgagactaaggacaacaaagaggacatcaagatcctgatcgcctctccaagcagcacccaggtgccacaagagacaaccacagccaaggcctctgcttacagcggcacccacagcagaaccgcttctccagatagagccggcaagagagtgatcgagcagaccgacaaggctcaccctagaagcctgaacctgagcgccacactgaaccagagaaacaccgtgcctgaggaagaactgaaccccaagacaatcgccagccagaacgcccagagaaagcggaagatggaacacgacggcagcctgttccaggctgctggaatcggaacactgcttcagcagccaggcgactgtgcccctacaatgagcctgagctggaagcgcgtgaagggcttcatcagctctgagcagaacggcaccgagcagaaaaccatcattctgatccccagcgacctggcctgtagactgctgggccaatctatggatgagagcggcctgcctcagctgaccagctacgactgtgaagtgaacgcccctatccagggcagcagaaacctgctccagggcgaagaactgctgagagccctggaccaagtgaactga

SEQ ID NO: 2 - protein, 836 aa, Mus musculus
MEGAGGENEKKKMSSERRKEKSRDAARSRRSKESEVFYELAHQLPLPHNVSSHLDKASVMRLTISYLRVRKLLDAGGLDSEDEMKAQMDCFYLKALDGFVMVLTDDGDMVYISDNVNKYMGLTQFELTGHSVFDFTHPCDHEEMREMLTHRNGPVRKGKELNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPSSETPKPLRSSADPALNQEVALKLESSPESLGLSFTMPQIQDQPASPSDGSTRQSSPERLLQENVNTPNFSQPNSPSEYCFDVDSDMVNVFKLELVEKLFAEDTEAKNPFSTQDTDLDLEMLAPYIPMDDDFQLRSFDQLSPLESNSPSPPSMSTVTGFQQTQLQKPTITATATTTATTDESKTETKDNKEDIKILIASPSSTQVPQETTTAKASAYSGTHSRTASPDRAGKRVIEQTDKAHPRSLNLSATLNQRNTVPEEELNPKTIASQNAQRKRKMEHDGSLFQAAGIGTLLQQPGDCAPTMSLSWKRVKGFISSEQNGTEQKTIILIPSDLACRLLGQSMDESGLPQLTSYDCEVNAPIQGSRNLLQGEELLRALDQVN

SEQ ID NO: 3 - DNA, 2625 bp, Mus musculus
  source 1..2625: mol_type = other DNA
atgacagccgacaaagagaagaagagaagctccagcgagctgcggaaagagaagtctagggacgccgccagatgcagaagatccaaagaaaccgaggtgttctacgagctggcccacgaactgcctctgcctcactctgtgtctagccacctggacaaggccagcatcatgagactggctatcagcttcctgagaacccacaagctgctgtccagcgtgtgcagcgagaacgagtctgaggccgaagccgaccagcagatggacaacctgtacctgaaggccctggaaggctttatcgccgtggtcacacaggacggcgatatgatcttcctgagcgagaacatcagcaagttcatgggcctgacacaggtggaactgaccggccacagcatcttcgacttcacacacccctgcgaccacgaggaaatcagagagaacctgacactgaagaacggctctggcttcggcaagaagtccaaggatgtgtctaccgagagggacttcttcatgcggatgaagtgtaccgtgaccaacagaggcagaaccgtgaacctgaagtccgccacatggaaggtgctgcactgtaccggacaagtgcgggtctacaacaactgccctcctcacagcagcctgtgcggctctaaagagcctctgctgagctgcctgatcatcatgtgcgagcccatccagcatccttctcacatggacatccctctggactccaagaccttcctgtccagacacagcatggacatgaagttcacctactgcgacgaccgcatcctggaactgatcggatatcaccccgaggaactgctgggcagaagcgcctacgagttctaccacgctctggattccgagaacatgaccaagagccaccagaacctgtgtaccaaaggccaggtggtgtccggccagtacagaatgctggctaagcacggcggctacgtgtggctggaaacacagggcaccgtgatctacaaccccagaaacctccagcctcagtgcattatgtgcgtgaactacgtgctgtccgagatcgagaagaacgacgtggtgttcagcatggatcagaccgagagcctgttcaagccccacctgatggccatgaactctatcttcgacagcagcgacgacgtggccgtgaccgaaaagagcaactacctgttcaccaagctgaaagaggaacctgaggaactggcccagctggctcctacacctggcgacgctatcatcagcctggacttcggcagccagaacttcgacgagccttctgcctacggcaaggccatccttccacctgggcaaccttgggtttcaggactgagaagccacagcgcccagagcgagtctggatctctgcctgctttcaccgtgcctcaggccgacacaccaggcaacacaacacctagcgccagcagcagctccagctgttctacacctagcagccccgaggactactacagctccctggaaaaccctctgaagatcgaagtcatcgagaagctgttcgctatggacaccgagcctagagatcctggcagcacccagacagacttctccgagctggacctggaaaccctggctccttacatcccaatggacggcgaggacttccagctgagccctatctgtccagaggaacccctgatgcctgagagccctcagcctacacctcagcactgcttcagcaccatgacctccatcttccagcctctgacacccggcgctacacacggcccattctttctggacaagtaccctcagcagctggaaagcagaaagaccgagtccgagcactggcctatgtctagcatcttcttcgacgccggcagcaagggcagcctgtctccttgttgtggccaggcctctacacccctgtcctctatgggcggcagatccaacacacagtggcctccagatcctcctctgcacttcggccctacaaaatggcctgtgggcgatcagtctgccgaaagccttggagcactgcctgtgggatcttcccagctggaacctccttctgctcctccacacgtgtccatgttcaagatgagaagcgccaaggactttggcgctagaggcccctacatgatgagccctgctatgatcgccctgagcaacaagctgaagctgaagagacagctggaatacgaggaacaggctttccaggacaccagcggcggagatccacctggcacatctagctctcacctgatgtggaagaggatgaagtccctgatgggcggaacatgccctctgatgcccgacaagacaatcagcgccaacatggcccctgacgagttcacccagaaatccatgagaggactgggccagccactgagacatctgcctccacctcaacctccaagcaccagaagctctggcgagaacgccaagacaggcttcccacctcagtgttacgccagtcagtttcaggattacggccctcctggcgctcagaaagtgtctggcgttgcaagcagactgctgggacctagcttcgagccttacctgctgcctgagctgaccagatacgactgcgaagtgaacgtgccagtgcctggcagctctacacttctgcaaggcagggatctgctgcgcgcactggatcaggctacatga

SEQ ID NO: 4 - protein, 874 aa, Mus musculus
MTADKEKKRSSSELRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLTLKNGSGFGKKSKDVSTERDFFMRMKCTVTNRGRTVNLKSATWKVLHCTGQVRVYNNCPPHSSLCGSKEPLLSCLIIMCEPIQHPSHMDIPLDSKTFLSRHSMDMKFTYCDDRILELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSDDVAVTEKSNYLFTKLKEEPEELAQLAPTPGDAIISLDFGSQNFDEPSAYGKAILPPGQPWVSGLRSHSAQSESGSLPAFTVPQADTPGNTTPSASSSSSCSTPSSPEDYYSSLENPLKIEVIEKLFAMDTEPRDPGSTQTDFSELDLETLAPYIPMDGEDFQLSPICPEEPLMPESPQPTPQHCFSTMTSIFQPLTPGATHGPFFLDKYPQQLESRKTESEHWPMSSIFFDAGSKGSLSPCCGQASTPLSSMGGRSNTQWPPDPPLHFGPTKWPVGDQSAESLGALPVGSSQLEPPSAPPHVSMFKMRSAKDFGARGPYMMSPAMIALSNKLKLKRQLEYEEQAFQDTSGGDPPGTSSSHLMWKRMKSLMGGTCPLMPDKTISANMAPDEFTQKSMRGLGQPLRHLPPPQPPSTRSSGENAKTGFPPQCYASQFQDYGPPGAQKVSGVASRLLGPSFEPYLLPELTRYDCEVNVPVPGSSTLLQGRDLLRALDQAT

SEQ ID NO: 5 - DNA, 2376 bp, Mus musculus
  source 1..2376: mol_type = other DNA
atggccgccaccaccgccaatcctgagatgacctctgatgtgcccagcctgggacctacaatcgcctctggaaatcccggacctggcattcaaggtggcggagctgttgtgcagagagccatcaagagaagaagcggcctggacttcgacgacgaggtggaagtgaacaccaagtttctgagatgcgacgacgaccagatgtgcaacgacaaagagagattcgccagatccgacgatgagcagagcagcgccgacaaagaaaggctggccagagagaaccacagcgagatcgagagaaggcgcagaaacaagatgaccgcctacatcaccgagctgagcgacatggtgcctacctgttctgccctggccagaaagcctgacaagctgaccatcctgagaatggccgtgtctcacatgaagtccctgagaggcaccggcaacacaagcacagacggcagctacaagcccagcttcctgaccgaccaagagctgaagcacctgatcctggaagccgccgatggcttcctgttcatcgtgtcttgcgagacaggcagagtggtgtatgtgtccgacagcgtgacccctgtgctgaaccagcctcagtctgagtggttcggcagcaccctgtacgaccaggttcaccctgacgatgtggataagctgagagagcagctcagcaccagcgagaacgctctgactggcagagtgctggacctgaaaaccggcaccgtgaagaaagagggccagcagtccagcatgagaatgtgcatgggcagccgtagatccttcatctgccggatgagatgcggcacctctagcgtggaccctgtgtctatgaacagactgtccttcctgagaaacagatgcagaaacggcctgggcagcgtgaaagaaggcgagcctcacttcgtggtggtgcattgcaccggctacatcaaggcttggcctccagccggtgtcagcctgcctgatgatgatcctgaagctggacagggcagcaagttctgcctggtggctatcggcagactgcaagtgacaagcagccccaactgcaccgacatgagcaacatctgccagcctaccgagttcatcagcagacacaacatcgagggcatcttcaccttcgtggaccacagatgtgtggccaccgtgggctatcagcctcaagagctgctgggcaagaacatcgtcgagttctgtcaccccgaggatcagcagcttctgagggacagctttcagcaggtcgtgaagctgaaaggccaggtgctgtccgtgatgttcagattcagatccaagaccagagaatggctgtggatgagaacaagcagcttcacatttcagaacccctactccgatgagatcgagtacatcatctgcaccaacaccaacgtgaagaacagcagccaagagcctagacctacactgagcaacacaatccccagaagccagctcggccctacagccaacctgagtctggaaatgggcaccggacagctccctagcagacagcagcagcaacagcacaccgaactggatatggtgcccggcagagatggcctggccagctacaatcactctcaggtgtcagtgcagcctgtggcctctgctggaagcgagcatagcaagcccctggaaaagagcgagggcctgttcgcccaggacagggaccctagattccccgaaatctaccccagcatcaccgccgaccagagcaagggcatcagctctagtacagtgcccgccacacagcagcttttcagccagggctctagcttcccacctaatcctagacctgccgagaacttcagaaacagcggcctgacacctcctgtgaccatcgtgcagccttctagctctgccggacagatcctggctcagatcagcaggcacagcaaccctgctcagggctctgctcctacctggacctcttctagcagacctggcttcgccgctcagcaggttccaacacaggctaccgccaagacaagatccagccagttcggcgtgaacaacttccagaccagctccagcttctccgccatgtctttgcctggcgctcctacagcctcttctggcacagcagcataccccgctctgcctaacagaggcagcaacttcccaccagagacaggccagacaaccggccagttccaggctagaacagccgaaggtgttggcgtgtggcctcagtggcaaggacagcagcctcaccacagaagcagctctagcgagcagcacgtgcagcagactcaagcccaggctccatctcagcccgaggtgttccaagagatgctgtccatgctgggcgaccagtccaacacctacaacaacgaagagttccccgacctgacaatgttccctccattcagcgaatag

SEQ ID NO: 6 - protein, 791 aa, Mus musculus
MAATTANPEMTSDVPSLGPTIASGNPGPGIQGGGAVVQRAIKRRSGLDFDDEVEVNTKFLRCDDDQMCNDKERFARSDDEQSSADKERLARENHSEIERRRRNKMTAYITELSDMVPTCSALARKPDKLTILRMAVSHMKSLRGTGNTSTDGSYKPSFLTDQELKHLILEAADGFLFIVSCETGRVVYVSDSVTPVLNQPQSEWFGSTLYDQVHPDDVDKLREQLSTSENALTGRVLDLKTGTVKKEGQQSSMRMCMGSRRSFICRMRCGTSSVDPVSMNRLSFLRNRCRNGLGSVKEGEPHFVVVHCTGYIKAWPPAGVSLPDDDPEAGQGSKFCLVAIGRLQVTSSPNCTDMSNICQPTEFISRHNIEGIFTFVDHRCVATVGYQPQELLGKNIVEFCHPEDQQLLRDSFQQVVKLKGQVLSVMFRFRSKTREWLWMRTSSFTFQNPYSDEIEYIICTNTNVKNSSQEPRPTLSNTIPRSQLGPTANLSLEMGTGQLPSRQQQQQHTELDMVPGRDGLASYNHSQVSVQPVASAGSEHSKPLEKSEGLFAQDRDPRFPEIYPSITADQSKGISSSTVPATQQLFSQGSSFPPNPRPAENFRNSGLTPPVTIVQPSSSAGQILAQISRHSNPAQGSAPTWTSSSRPGFAAQQVPTQATAKTRSSQFGVNNFQTSSSFSAMSLPGAPTASSGTAAYPALPNRGSNFPPETGQTTGQFQARTAEGVGVWPQWQGQQPHHRSSSSEQHVQQTQAQAPSQPEVFQEMLSMLGDQSNTYNNEEFPDLTMFPPFSE

SEQ ID NO: 7 - DNA, 2481 bp, Homo sapiens
  source 1..2481: mol_type = other DNA
atggaaggcgctggcggagccaacgacaagaagaagattagcagcgagcggcggaaagagaagtccagggacgctgccagaagcaggcggagcaaagaaagcgaggtgttctacgagctggcccaccaactgcctctgcctcacaatgtgtctagccacctggacaaggccagcgtgatgagactgaccatcagctacctgagagtgcggaagctgctggatgctggcgacctggacatcgaggacgatatgaaggcccagatgaactgcttctacctgaaggccctggacggcttcgtgatggtgctgaccgatgacggcgacatgatatacattagcgacaacgtgaacaagtatatggggctgacccagttcgagctgaccggccactccgtgttcgacttcacacacccttgcgaccacgaagagatgagagagatgctgacccaccggaacggcctggtcaagaagggcaaagagcagaatacccagcggtcattcttcctgcggatgaaatgcaccctgaccagccggggcagaaccatgaatatcaagagcgccacttggaaagtgctgcactgcaccggccacatccatgtgtatgacaccaacagcaaccagcctcagtgcggctacaagaaacctcctatgacctgcctggtgctgatttgcgagcccattcctcatcctagcaatatcgagattcccctggacagcaagaccttcctgagcagacacagcctggacatgaagtttagctactgcgacgagagaatcaccgagctgatgggctacgagcccgaggaacttctcggacggtccatctacgagtattatcacgccctggactccgaccacctgaccaagacacaccacgatatgttcaccaagggccaagtgaccaccggccagtatagaatgctggctaagcgcggaggctatgtgtgggttgagacacaggccaccgtcatctacaacaccaagaactcccagccacagtgcatcgtgtgcgtgaactacgtcgtgtccggcatcatccagcacgacctgattttcagcctgcaacagaccgagtgcgtgctgaagcctgtggaaagcagcgacatgaagatgacccagctttttaccaaggtggaatccgaggacaccagcagcctgttcgacaagctgaagaaagagcccgacgctctgacactgctggctccagcagcaggcgataccatcatcagcctggatttcggcagcaacgacaccgagacagacgaccagcagctcgaagaggtgcccctgtataacgatgtgatgctgccctctccaaacgagaagctccagaacatcaacctggctatgagccctctgcctaccgccgagacacctaagcctctgagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgatatggtcaatgagttcaaactggaactggtcgagaagctgttcgccgaagataccgaggccaagaatcccttcagcacccaagacactgacctggacctggaaatgctggccccttacatccctatggacgacgactttcagctccggtccttcgaccagctcagccctctggaatccagctctgcctctccagaaagcgcctctccacagagcaccgtgaccgtgtttcagcagacccagattcaagagcccaccgccaatgccaccaccacaaccgccacaacagacgagctgaaaaccgtgaccaaggaccgcatggaggacatcaagattctgattgcctctccttctccgacgcacatccacaaagagacaaccagcgccacaagcagcccctacagagacacccagagcagaaccgcctctcctaatagagccggaaagggcgtcatcgagcagaccgagaagtctcaccctagaagccccaacgtgctgagcgtggcactgagccagagaaccacagtgcccgaagaggaactgaaccctaagattctggccctccagaacgcccagcggaagcggaaaatggaacacgacggaagcctgttccaggccgtcggaatcggaacactgctgcaacagcctgatgaccacgccgccaccacaagcctgtcttggaagagagtgaagggctgcaagtccagcgagcagaacggaatggaacagaaaaccatcatcctgattcctagcgacctggcctgtagactgctgggccagagcatggatgagagcggactgccacagctcaccagctacgactgcgaagtgaacgcccctatccagggcagcagaaacctgctccaaggcgaggaactgctgcgagcactggaccaagtgaactga

SEQ ID NO: 8 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 9 - DNA, 2613 bp, Homo sapiens
  source 1..2613: mol_type = other DNA
atgaccgccgacaaagagaagaagagaagctccagcgagcggcggaaagagaagtctagggacgccgccagatgcaggcggagcaaagaaaccgaggtgttctacgagctggcccacgaactgcctctgcctcactctgtgtctagccacctggacaaggccagcatcatgcggctggctatcagcttcctgcggacccacaaactgctgagcagcgtgtgcagcgagaatgagtctgaagccgaagccgaccagcagatggacaacctgtatctgaaggccctggaaggcttcattgccgtcgtgacccaggacggcgatatgattttcctgagcgagaacatcagcaagttcatgggcctgacacaggtggaactgaccggccacagcatcttcgacttcacacacccctgcgaccacgaggaaatcagagagaacctgagcctgaagaacggcagcggctttggcaagaaaagcaaggatatgagcaccgagcgggatttcttcatgcggatgaaatgcaccgtgaccaaccggggcagaaccgtgaatctgaaatccgccacatggaaggtgctgcactgcaccggccaagtgaaagtgtataacaactgccctccacacaacagcctgtgcggctacaaagagcctctgctgagctgcctgattatcatgtgcgagcccattcagcacccctctcacatggacatccctctggacagcaagaccttcctgagcaggcacagcatggacatgaagttcacctactgcgacgaccgcatcaccgagctgattggctatcaccccgaagaactgctgggcagaagcgcctacgagttctatcacgccctggactccgagaacatgaccaagagccaccagaacctctgcaccaaaggccaggtggtgtccggccagtatcggatgcttgccaagcacggcggctatgtgtggctggaaacacagggcaccgtcatctacaaccccagaaacctccagcctcagtgcattatgtgcgtgaactacgtgctgagcgaaatcgagaagaacgatgtggtgtttagcatggaccagaccgagagcctgttcaagccccacctgatggctatgaactccatcttcgatagcagcggcaagggcgctgtgtccgagaagtccaacttcctgttcaccaagctgaaagaggaacccgaggaactggcccagcttgctcctacacctggcgacgccatcatcagcctggacttcggcaaccagaacttcgaggaaagctccgcctacggcaaggccatcctgcctccatctcaaccttgggccacagagctgagaagccacagcacacagagcgaagctggaagcctgcctgcctttacagtgccacaagctgctgcccctggcagcacaacaccaagcgcaacaagcagcagcagctcttgcagcaccccaaacagccccgaggactactacaccagcctggataacgacctgaagattgaagtcatcgagaagctgttcgctatggacaccgaggccaaggaccagtgtagcacccagaccgacttcaacgagctggacctggaaactctggccccttacatcccaatggacggcgaggatttccagctcagccctatctgccccgaagagagactgctggccgagaatcctcagagcaccccacagcactgcttcagcgccatgaccaacatcttccagcctctcgctccagtggctccacacagcccatttctgctggacaagttccagcagcaactcgaaagcaagaaaaccgagcctgagcaccggcctatgagcagcatctttttcgacgccggaagcaaagccagcctgccaccttgttgtggacaggcttctacccctctgtccagcatgggcggcaggtctaatactcagtggcctcctgaccctccactgcactttggccctacaaaatgggcagtgggcgaccagagaaccgagtttcttggagctgctccactgggaccaccagtgtctccacctcatgtgtccaccttcaagacaagaagcgccaaaggcttcggcgctagaggccctgatgtgctgtctcctgctatggtggccctgagcaacaagctgaagctgaagagacagctcgaatacgaggaacaggctttccaggacctcagtggtggcgaccctcctggtggaagcacatctcacctgatgtggaagcggatgaagaatctgagaggcggctcctgtcctctgatgcccgataagcctctgagcgccaacgtgcccaacgacaagttcacacagaaccccatgagaggactgggacaccctctgaggcatctgccattgccacagcctccaagcgccatttctcccggcgagaacagcaagagcagattccctccacagtgctacgccacacagtatcaggactacagcctgtctagcgcccacaaagtgtccggcatggcctcaagactgctcggccctagcttcgagagctacctgctgcctgagctgaccagatacgactgcgaagtgaacgtgcccgtgctgggctctagcacacttcttcaaggcggcgacctgctgagagcactggaccaggcaacatga

SEQ ID NO: 10 - protein, 870 aa, Homo sapiens
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTLLQGGDLLRALDQAT

SEQ ID NO: 11 - protein, 9 aa, synthetic construct
  VARIANT 4: note = X can be any amino acid
  VARIANT 5: note = X can be any amino acid
  VARIANT 6: note = X can be any amino acid
QQQXXXQQQ

SEQ ID NO: 12 - protein, 9 aa, synthetic construct
  VARIANT 4: note = X can be any amino acid
  VARIANT 5: note = X can be any amino acid
  VARIANT 6: note = X can be any amino acid
PPPXXXPPP

SEQ ID NO: 13 - protein, 6 aa, synthetic construct
  VARIANT 3: note = X can be any amino acid
  VARIANT 4: note = X can be any amino acid
IIXXII

SEQ ID NO: 14 - DNA, 266 bp, synthetic construct
  source 1..266: mol_type = other DNA
ctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatg

SEQ ID NO: 15 - DNA, 582 bp, synthetic construct
  source 1..582: mol_type = other DNA
atggactacaaagaccacgacggcgattacaaggatcatgacatcgattacaaagacgatgacgataagatggctcctaagaaaaagcgcaaagtctccggagacgctctggatgactttgatctggacatgcttggttccgatgccttggacgattttgacctcgatatgctgggctccgacgctctcgacgatttcgacctcgacatgctgggtagcgatgcactggatgactttgatctggatatgctcggatccggtggtggcgggtctggaggaggaggtagtggtggaggtggctctggtaccgccagacctggagaaagacctttccagtgcagaatctgtatgagaaacttcagcagacaggaccgcctggacagacacaccagaacacatacaggagagaagccatttcagtgtaggatctgtatgaggaactttagccagaaggagcacctggccggacatctgcgcacacacactggagagaagccattccagtgtagaatctgcatgagaaacttcagcagacgcgacaacctgaacagacacctcaagacacacctgagaggaagctga

SEQ ID NO: 16 - DNA, 768 bp, synthetic construct
  source 1..768: mol_type = other DNA
atggactacaaagaccacgacggcgattacaaggatcatgacatcgattacaaagacgatgacgataagatggctcctaagaaaaagcgcaaagtctccggaggaggtctggacctggagatgctggctccatacatcccaatggatgatgacttccagctgggaggtggagacgctctggatgactttgatctggacatgcttggttccgatgccttggacgattttgacctcgatatgctgggctccgacgctctcgacgatttcgacctcgacatgctgggtagcgatgcactggatgactttgatctggatatgctcggatccggtggtggcgggagtctggacctggagatgctggctccatacatcccaatggatgatgacttccagctgggaggaggaggtagtggtggaggtggctctggtaccgccagacctggagaaagacctttccagtgcagaatctgtatgagaaacttcagcagacaggaccgcctggacagacacaccagaacacatacaggagagaagccatttcagtgtaggatctgtatgaggaactttagccagaaggagcacctggccggacatctgcgcacacacactggagagaagccattccagtgtagaatctgcatgagaaacttcagcagacgcgacaacctgaacagacacctcaagacacacctgagaggaagcggaggaggtctggacctggagatgctggctccatacatcccaatggatgatgacttccagctgtga

SEQ ID NO: 17 - DNA, 255 bp, synthetic construct
  source 1..255: mol_type = other DNA
atatacgcgtagagtgaggacgccgagagtgaggacaaggagagtgaggacctgaagagtgaggacactcagagtgaggacgcgaagagtgaggacaggaagagtgaggacggaaagagtgaggacagtcagagtgaggacgctgagagtgaggacatcaagagtgaggactaaaagagtgaggacacgctctctagagggtatataatgggggccactagtctactaccagagctcatcgctagcgccaccatg

SEQ ID NO: 18 - DNA, 475 bp, synthetic construct
  source 1..475: mol_type = other DNA
acgggccagatatacgcgtagagtgaggacgccgagagtgaggacaaggagagtgaggacctgaagagtgaggacactcagagtgaggacgcgaagagtgaggacaggaagagtgaggacggaaagagtgaggacagtcagagtgaggacgctgagagtgaggacatcaagagtgaggactaaaagagtgaggaccaggtagtgtacatctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatg

SEQ ID NO: 19 - DNA, 2380 bp, synthetic construct
  source 1..2380: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatgggccctggcgttctgctgcttctgctggttgctacagcctggcatggacaaggcggagtggtgtcccacttcaacgattgccctctgagccacgacggctactgtctgcatgatggcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagaggcagggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagagcaccagcagcaggcgataccatcatcagcctggacttcggcagcaacgacaccgagacagacgaccagcagctcgaagaggtgcccctgtataatgatgtgatgctgccctctccaaatgagaagctccagaacatcaacctggctatgagccctctgcctaccgccgagacacctaagcctctcagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgacatggtcaatgagttcaaactggaactggtcgagaagctgttcgccgaggacaccgaggccaagaatcccttcagcacccaagacaccgacctggacctggaaatgctggccccttacatccctatggacgacgacttccagctccggtccttcgaccagctcagccctctggaaagctctagcgcctctcctgaatccgcctctccacagagcacagtgaccgtgttccaatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataagctctagagggacagcccccccccaaagcccccagggatgtaattacgtccctcccccgctagggggcagcagcgagccgcccggggctccgctccggtccggcgctccccccgc

SEQ ID NO: 20 - DNA, 2940 bp, synthetic construct
  source 1..2940: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagagctcgtttagtgaaccgtcagatcgcctggagacgccatccacgctgttttgacctccatagaagagaacgggaccgatccaaattctcgaggctagcgccaccatgggccctggcgttctgctgcttctgctggttgctacagcctggcatggacaaggcggagtggtgtcccacttcaacgattgccctctgagccacgacggctactgtctgcatgatggcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagaggcagggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataagctctagagggacagcccccccccaaagcccccagggatgtaattacgtccctcccccgctagggggcagcagcgagccgcccggggctccgctccggtccggcgc

SEQ ID NO: 21 - DNA, 1680 bp, synthetic construct
  source 1..1680: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatgggccctggcgttctgctgcttctgctggttgctacagcctggcatggacaaggcggagtggtgtcccacttcaacgattgccctctgagccacgacggctactgtctgcatgatggcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagaggcagggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggatggggggggcaggacagcaagggggaggattgggaagtcaatagcaggcatgctggggatgcggtgggctctatggcttctgaggcaggtcgagggtacatgcgccttactccttgtgagcgcatcgcgataagctctagagggacagccccccccc

SEQ ID NO: 22 - DNA, 3500 bp, synthetic construct
  source 1..3500: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagagctcgtttagtgaaccgtcagatcgcctggagacgccatccacgctgttttgacctccatagaagagaacgggaccgatccaaattctcgaggctagcgccaccatgggccctggcgttctgctgcttctgctggttgctacagcctggcatggacaaggcggagtggtgtcccacttcaacgattgccctctgagccacgacggctactgtctgcatgatggcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagaggcagggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagagcaccagcagcaggcgataccatcatcagcctggacttcggcagcaacgacaccgagacagacgaccagcagctcgaagaggtgcccctgtataatgatgtgatgctgccctctccaaatgagaagctccagaacatcaacctggctatgagccctctgcctaccgccgagacacctaagcctctcagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgacatggtcaatgagttcaaactggaactggtcgagaagctgttcgccgaggacaccgaggccaagaatcccttcagcacccaagacaccgacctggacctggaaatgctggccccttacatccctatggacgacgacttccagctccggtccttcgaccagctcagccctctggaaagctctagcgcctctcctgaatccgcctctccacagagcacagtgaccgtgttccaatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataagctctagagggacagcccccccccaaagcccccagggatgtaattacgtccctcccc

SEQ ID NO: 23 - DNA, 1260 bp, synthetic construct
  source 1..1260: mol_type = other DNA
ggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaagccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactcacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 24 - DNA, 700 bp, synthetic construct
  source 1..700: mol_type = other DNA
taattctccttggaatttgccctttttgagtttggatcttggttcattctcaagcctcagacagtggttcaaagtttttttcttccatttcaggtgtcgtgagagctcgtttagtgaaccgtcagatcgcctggagacgccatccacgctgttttgacctccatagaagagaacgggaccgatccaaattctcgaggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataagctctagagggacagcccccccccaaagcccccagggatgtaattacgtccctcccccgctagggggcagcagcgagccgcccggggct

SEQ ID NO: 25 - DNA, 4900 bp, synthetic construct
  source 1..4900: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatggactacaaggaccacgacggcgattacaaagaccatgacatcgactataaggacgatgacgacaaaggcagcggcatggaaggcgctggcggagccaacgacaagaagaagattagcagcgagcggcggaaagagaagtccagggacgctgccagaagcaggcggagcaaagaaagcgaggtgttctacgagctggcccaccaactgcctctgcctcacaatgtgtctagccacctggacaaggccagcgtgatgagactgaccatcagctacctgagagtgcggaagctgctggatgctggcgacctggacatcgaggacgatatgaaggcccagatgaactgcttctacctgaaggccctggacggcttcgtgatggtgctgaccgatgacggcgacatgatatacattagcgacaacgtgaacaagtatatggggctgacccagttcgagctgaccggccactccgtgttcgacttcacacacccttgcgaccacgaagagatgagagagatgctgacccaccggaacggcctggtcaagaagggcaaagagcagaatacccagcggtcattcttcctgcggatgaaatgcaccctgaccagccggggcagaaccatgaatatcaagagcgccacttggaaagtgctgcactgcaccggccacatccatgtgtatgacaccaacagcaaccagcctcagtgcggctacaagaaacctcctatgacctgcctggtgctgatttgcgagcccattcctcatcctagcaatatcgagattcccctggacagcaagaccttcctgagcagacacagcctggacatgaagtttagctactgcgacgagagaatcaccgagctgatgggctacgagcccgaggaacttctcggacggtccatctacgagtattatcacgccctggactccgaccacctgaccaagacacaccacgatatgttcaccaagggccaagtgaccaccggccagtatagaatgctggctaagcgcggaggctatgtgtgggttgagacacaggccaccgtcatctacaacaccaagaactcccagccacagtgcatcgtgtgcgtgaactacgtcgtgtccggcatcatccagcacgacctgattttcagcctgcaacagaccgagtgcgtgctgaagcctgtggaaagcagcgacatgaagatgacccagctttttaccaaggtggaatccgaggacaccagcagcctgttcgacaagctgaagaaagagcccgacgctctgacactgctggctccagcagcaggcgataccatcatcagcctggatttcggcagcaacgacaccgagacagacgaccagcagctcgaagaggtgcccctgtataacgatgtgatgctgccctctccaaacgagaagctccagaacatcaacctggctatgagccctctgcctaccgccgagacacctaagcctctgagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgatatggtcaatgagttcaaactggaactggtcgagaagctgttcgccgaagataccgaggccaagaatcccttcagcacccaagacactgacctggacctggaaatgctggccccttacatccctatggacgacgactttcagctccggtccttcgaccagctcagccctctggaatccagctctgcctctccagaaagcgcctctccacagagcaccgtgaccgtgtttcagcagacccagattcaagagcccaccgccaatgccaccaccacaaccgccacaacagacgagctgaaaaccgtgaccaaggaccgcatggaggacatcaagattctgattgcctctccttctccgacgcacatccacaaagagacaaccagcgccacaagcagcccctacagagacacccagagcagaaccgcctctcctaatagagccggaaagggcgtcatcgagcagaccgagaagtctcaccctagaagccccaacgtgctgagcgtggcactgagccagagaaccacagtgcccgaagaggaactgaaccctaagattctggccctccagaacgcccagcggaagcggaaaatggaacacgacggaagcctgttccaggccgtcggaatcggaacactgctgcaacagcctgatgaccacgccgccaccacaagcctgtcttggaagagagtgaagggctgcaagtccagcgagcagaacggaatggaacagaaaaccatcatcctgattcctagcgacctggcctgtagactgctgggccagagcatggatgagagcggactgccacagctcaccagctacgactgcgaagtgaacgcccctatccagggcagcagaaacctgctccaaggcgaggaactgctgcgagcactggaccaagtgaaccggagaaaaagaagcggcagcggagaaggcagaggcagccttcttacatgcggagatgtggaagagaaccccggacctatgggccctggcgttctgctgcttctgctggttgctacagcctggcatggacaaggcggagtggtgtcccacttcaacgattgccctctgagccacgacggctactgtctgcatgatggcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagaggcagggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagagcaccagcagcaggcgataccatcatcagcctggacttcggcagcaacgacaccgagacagacgaccagcagctcgaagaggtgcccctgtataatgatgtgatgctgccctctccaaatgagaagctccagaacatcaacctggctatgagccctctgcctaccgccgagacacctaagcctctcagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgacatggtcaatgagttcaaactggaactggtcgagaagctgttcgccgaggacaccgaggccaagaatcccttcagcacccaagacaccgacctggacctggaaatgctggccccttacatccctatggacgacgacttccagctccggtccttcgaccagctcagccctctggaaagctctagcgcctctcctgaatccgcctctccacagagcacagtgaccgtgttccaatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataag

SEQ ID NO: 26 - DNA, 3780 bp, synthetic construct
  source 1..3780: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatgggccctggcgttctgctgcttctgctggttgctacagcctggcatggacaaggcggagtggtgtcccacttcaacgattgccctctgagccacgacggctactgtctgcatgatggcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagaggcagggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagagcaccagcagcaggcgataccatcatcagcctggacttcggcagcaacgacaccgagacagacgaccagcagctcgaagaggtgcccctgtataatgatgtgatgctgccctctccaaatgagaagctccagaacatcaacctggctatgagccctctgcctaccgccgagacacctaagcctctcagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgacatggtcaatgagttcaaactggaactggtcgagaagctgttcgccgaggacaccgaggccaagaatcccttcagcacccaagacaccgacctggacctggaaatgctggccccttacatccctatggacgacgacttccagctccggtccttcgaccagctcagccctctggaaagctctagcgcctctcctgaatccgcctctccacagagcacagtgaccgtgttccaacggagaaaaagaagcggcagcggagaaggcagaggcagccttcttacatgcggagatgtggaagagaaccccggacctatggactacaaggaccacgacggcgattacaaagaccatgacatcgactataaggacgatgacgacaaaggcagcggcatggaaggcgctggcggagccaacgacaagaagaagattagcagcgagcggcggaaagagaagtccagggacgctgccagaagcaggcggagcaaagaaagcgaggtgttctacgagctggcccaccaactgcctctgcctcacaatgtgtctagccacctggacaaggccagcgtgatgagactgaccatcagctacctgagagtgcggaagctgctggatgctggcgacctggacatcgaggacgatatgaaggcccagatgaactgcttctacctgaaggccctggacggcttcgtgatggtgctgaccgatgacggcgacatgatatacattagcgacaacgtgaacaagtatatggggctgacccagttcgagctgaccggccactccgtgttcgacttcacacacccttgcgaccacgaagagatgagagagatgctgacccaccggaacggcctggtcaagaagggcaaagagcagaatacccagcggtcattcttcctgcggatgaaatgcaccctgaccagccggggcagaaccatgaatatcaagagcgccacttggaaagtgctgcactgcaccggccacatccatgtgtatgacaccaacagcaaccagcctcagtgcggctacaagaaacctcctatgacctgcctggtgctgatttgcgagcccattcctcatcctagcaatatcgagattcccctggacagcaagaccttcctgagcagacacagcctggacatgaagtttagctactgcgacgagagaatcaccgagctgatgggctacgagcccgaggaacttctcggacggtccatctacgagtattatcacgccctggactccgaccacctgaccaagacacaccacgatatgttcaccaagggccaagtgaccaccggccagtatagaatgctggctaagcgcggaggctatgtgtgggttgagacacaggccaccgtcatctacaacaccaagaactcccagccacagtgcatcgtgtgcgtgaactacgtcgtgtccggcatcatccagcacgacctgattttcagcctgcaacagaccgagtgcgtgctgaagcctgtggaaagcagcgacatgaagatgacccagctttttaccaaggtggaatccgaggacaccagcagcctgttcgacaagctgaagaaagagcccgacgctctgacactgctggctccagcagcaggcgataccatcatcagcctggatttcggcagcaacgacaccgagacagacgaccagcagctcgaagaggtgcccctgtataacgatgtgatgctgccctctccaaacgagaagctccagaacatcaacctggctatgagccctctgcctaccgccgagacacctaagcctctgagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgatatggtcaatgagttcaaactggaactggtcgagaagctgttcgccgaagataccgaggccaagaatcccttcagcacccaagacactgacctggacctggaaatgctggcccctta

SEQ ID NO: 27 - DNA, 980 bp, synthetic construct
  source 1..980: mol_type = other DNA
ccaatgccaccaccacaaccgccacaacagacgagctgaaaaccgtgaccaaggaccgcatggaggacatcaagattctgattgcctctccttctccgacgcacatccacaaagagacaaccagcgccacaagcagcccctacagagacacccagagcagaaccgcctctcctaatagagccggaaagggcgtcatcgagcagaccgagaagtctcaccctagaagccccaacgtgctgagcgtggcactgagccagagaaccacagtgcccgaagaggaactgaaccctaagattctggccctccagaacgcccagcggaagcggaaaatggaacacgacggaagcctgttccaggccgtcggaatcggaacactgctgcaacagcctgatgaccacgccgccaccacaagcctgtcttggaagagagtgaagggctgcaagtccagcgagcagaacggaatggaacagaaaaccatcatcctgattcctagcgacctggcctgtagactgctgggccagagcatggatgagagcggactgccacagctcaccagctacgactgcgaagtgaacgcccctatccagggcagcagaaacctgctccaaggcgaggaactgctgcgagcactggaccaagtgaactgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataag

SEQ ID NO: 28 - DNA, 5040 bp, synthetic construct
  source 1..5040: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatggactacaaggaccacgacggcgattacaaagaccatgacatcgactataaggacgatgacgacaaaggcagcggcatgaccgccgacaaagagaagaagagaagctccagcgagcggcggaaagagaagtctagggacgccgccagatgcaggcggagcaaagaaaccgaggtgttctacgagctggcccacgaactgcctctgcctcactctgtgtctagccacctggacaaggccagcatcatgcggctggctatcagcttcctgcggacccacaaactgctgagcagcgtgtgcagcgagaatgagtctgaagccgaagccgaccagcagatggacaacctgtatctgaaggccctggaaggcttcattgccgtcgtgacccaggacggcgatatgattttcctgagcgagaacatcagcaagttcatgggcctgacacaggtggaactgaccggccacagcatcttcgacttcacacacccctgcgaccacgaggaaatcagagagaacctgagcctgaagaacggcagcggctttggcaagaaaagcaaggatatgagcaccgagcgggatttcttcatgcggatgaaatgcaccgtgaccaaccggggcagaaccgtgaatctgaaatccgccacatggaaggtgctgcactgcaccggccaagtgaaagtgtataacaactgccctccacacaacagcctgtgcggctacaaagagcctctgctgagctgcctgattatcatgtgcgagcccattcagcacccctctcacatggacatccctctggacagcaagaccttcctgagcaggcacagcatggacatgaagttcacctactgcgacgaccgcatcaccgagctgattggctatcaccccgaagaactgctgggcagaagcgcctacgagttctatcacgccctggactccgagaacatgaccaagagccaccagaacctctgcaccaaaggccaggtggtgtccggccagtatcggatgcttgccaagcacggcggctatgtgtggctggaaacacagggcaccgtcatctacaaccccagaaacctccagcctcagtgcattatgtgcgtgaactacgtgctgagcgaaatcgagaagaacgatgtggtgtttagcatggaccagaccgagagcctgttcaagccccacctgatggctatgaactccatcttcgatagcagcggcaagggcgctgtgtccgagaagtccaacttcctgttcaccaagctgaaagaggaacccgaggaactggcccagcttgctcctacacctggcgacgccatcatcagcctggacttcggcaaccagaacttcgaggaaagctccgcctacggcaaggccatcctgcctccatctcaaccttgggccacagagctgagaagccacagcacacagagcgaagctggaagcctgcctgcctttacagtgccacaagctgctgcccctggcagcacaacaccaagcgcaacaagcagcagcagctcttgcagcaccccaaacagccccgaggactactacaccagcctggataacgacctgaagattgaagtcatcgagaagctgttcgctatggacaccgaggccaaggaccagtgtagcacccagaccgacttcaacgagctggacctggaaactctggccccttacatcccaatggacggcgaggatttccagctcagccctatctgccccgaagagagactgctggccgagaatcctcagagcaccccacagcactgcttcagcgccatgaccaacatcttccagcctctcgctccagtggctccacacagcccatttctgctggacaagttccagcagcaactcgaaagcaagaaaaccgagcctgagcaccggcctatgagcagcatctttttcgacgccggaagcaaagccagcctgccaccttgttgtggacaggcttctacccctctgtccagcatgggcggcaggtctaatactcagtggcctcctgaccctccactgcactttggccctacaaaatgggcagtgggcgaccagagaaccgagtttcttggagctgctccactgggaccaccagtgtctccacctcatgtgtccaccttcaagacaagaagcgccaaaggcttcggcgctagaggccctgatgtgctgtctcctgctatggtggccctgagcaacaagctgaagctgaagagacagctcgaatacgaggaacaggctttccaggacctcagtggtggcgaccctcctggtggaagcacatctcacctgatgtggaagcggatgaagaatctgagaggcggctcctgtcctctgatgcccgataagcctctgagcgccaacgtgcccaacgacaagttcacacagaaccccatgagaggactgggacaccctctgaggcatctgccattgccacagcctccaagcgccatttctcccggcgagaacagcaagagcagattccctccacagtgctacgccacacagtatcaggactacagcctgtctagcgcccacaaagtgtccggcatggcctcaagactgctcggccctagcttcgagagctacctgctgcctgagctgaccagatacgactgcgaagtgaacgtgcccgtgctgggctctagcacacttcttcaaggcggcgacctgctgagagcactggaccaggcaacacggagaaaaagaagcggcagcggagaaggcagaggcagccttcttacatgcggagatgtggaagagaaccccggacctatgggccctggcgttctgctgcttctgctggttgctacagcctggcatggacaaggcggagtggtgtcccacttcaacgattgccctctgagccacgacggctactgtctgcatgatggcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagaggcagggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagagcaccagcagcaggcgataccatcatcagcctggacttcggcagcaacgacaccgagacagacgaccagcagctcgaagaggtgcccctgtataatgatgtgatgctgccctctccaaatgagaagctccagaacatcaacctggctatgagccctctgcctaccgccgagacacctaagcctctcagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgacatggtcaatgagttcaaactggaactggtcgagaagctgttcgccgaggacaccgaggccaagaatcccttcagcacccaagacaccgacctggacctggaaatgctggccccttacatccctatggacgacgacttccagctccggtccttcgaccagctcagccctctggaaagctctagcgcctctcctgaatccgcctctccacagagcacagtgaccgtgttccaatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataagctctagag

SEQ ID NO: 29 - DNA, 5040 bp, synthetic construct
  source 1..5040: mol_type = other DNA
ggtcttgacaaacgagtgctagtacaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatgggccctggcgttctgctgcttctgctggttgctacagcctggcatggacaaggcggagtggtgtcccacttcaacgattgccctctgagccacgacggctactgtctgcatggatgcgtgtgcatgtatatcgaggccctggacaaatacgcctgcaactgcgtcgtgggctacatcggcgagagatgccagtatcgggacctgaagtggtgggagctgagagccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttacagtggccttcatcatcttttgggtccgaagcaagcggagcagactgctgcacagcgactacatgaacatgacccctagacggcccggaccaaccagaaagcactaccagccttacgctccacctagagacttcgccgcctatcggagcagagtgaagttctccagaagcgccgacgctcctgcctatcagcagggacagaaccagctctacaacgagctgaacctggggcgcagagaagagtatgatgtgctggataagcggagagggcaggaccctgagatgggcggaaagcccagacggaagaatcctcaagagggcctgtataacgagctgcaaaaggacaagatggccgaagcctacagcgagattggcatgaagggcgaacgcagaagaggcaagggacacgacggcctgtatcagggactcagcaccgccaccaaggacacctatgatgccctgcacatgcaagccctgcctccaagagcaccagcagcaggcgataccatcatcagcctggacttcggcagcaacgacaccgagacagacgaccagcagctcgaagaggtgcccctgtataatgatgtgatgctgccctctccaaatgagaagctccagaacatcaacctggctatgagccctctgcctaccgccgagacacctaagcctctcagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgacatggtcaatgagttcaaactggaactggtcgagaagctgttcgccgaggacaccgaggccaagaatcccttcagcacccaagacaccgacctggacctggaaatgctggccccttacatccctatggacgacgacttccagctccggtccttcgaccagctcagccctctggaaagctctagcgcctctcctgaatccgcctctccacagagcacagtgaccgtgttccaacggagaaaaagaagcggcagcggagaaggcagaggcagccttcttacatgcggagatgtggaagagaaccccggacctatggactacaaggaccacgacggcgattacaaagaccatgacatcgactataaggacgatgacgacaaaggcagcggcatgaccgccgacaaagagaagaagagaagctccagcgagcggcggaaagagaagtctagggacgccgccagatgcaggcggagcaaagaaaccgaggtgttctacgagctggcccacgaactgcctctgcctcactctgtgtctagccacctggacaaggccagcatcatgcggctggctatcagcttcctgcggacccacaaactgctgagcagcgtgtgcagcgagaatgagtctgaagccgaagccgaccagcagatggacaacctgtatctgaaggccctggaaggcttcattgccgtcgtgacccaggacggcgatatgattttcctgagcgagaacatcagcaagttcatgggcctgacacaggtggaactgaccggccacagcatcttcgacttcacacacccctgcgaccacgaggaaatcagagagaacctgagcctgaagaacggcagcggctttggcaagaaaagcaaggatatgagcaccgagcgggatttcttcatgcggatgaaatgcaccgtgaccaaccggggcagaaccgtgaatctgaaatccgccacatggaaggtgctgcactgcaccggccaagtgaaagtgtataacaactgccctccacacaacagcctgtgcggctacaaagagcctctgctgagctgcctgattatcatgtgcgagcccattcagcacccctctcacatggacatccctctggacagcaagaccttcctgagcaggcacagcatggacatgaagttcacctactgcgacgaccgcatcaccgagctgattggctatcaccccgaagaactgctgggcagaagcgcctacgagttctatcacgccctggactccgagaacatgaccaagagccaccagaacctctgcaccaaaggccaggtggtgtccggccagtatcggatgcttgccaagcacggcggctatgtgtggctggaaacacagggcaccgtcatctacaaccccagaaacctccagcctcagtgcattatgtgcgtgaactacgtgctgagcgaaatcgagaagaacgatgtggtgtttagcatggaccagaccgagagcctgttcaagccccacctgatggctatgaactccatcttcgatagcagcggcaagggcgctgtgtccgagaagtccaacttcctgttcaccaagctgaaagaggaacccgaggaactggcccagcttgctcctacacctggcgacgccatcatcagcctggacttcggcaaccagaacttcgaggaaagctccgcctacggcaaggccatcctgcctccatctcaaccttgggccacagagctgagaagccacagcacacagagcgaagctggaagcctgcctgcctttacagtgccacaagctgctgcccctggcagcacaacaccaagcgcaacaagcagcagcagctcttgcagcaccccaaacagccccgaggactactacaccagcctggataacgacctgaagattgaagtcatcgagaagctgttcgctatggacaccgaggccaaggaccagtgtagcacccagaccgacttcaacgagctggacctggaaactctggccccttacatcccaatggacggcgaggatttccagctcagccctatctgccccgaagagagactgctggccgagaatcctcagagcaccccacagcactgcttcagcgccatgaccaacatcttccagcctctcgctccagtggctccacacagcccatttctgctggacaagttccagcagcaactcgaaagcaagaaaaccgagcctgagcaccggcctatgagcagcatctttttcgacgccggaagcaaagccagcctgccaccttgttgtggacaggcttctacccctctgtccagcatgggcggcaggtctaatactcagtggcctcctgaccctccactgcactttggccctacaaaatgggcagtgggcgaccagagaaccgagtttcttggagctgctccactgggaccaccagtgtctccacctcatgtgtccaccttcaagacaagaagcgccaaaggcttcggcgctagaggccctgatgtgctgtctcctgctatggtggccctgagcaacaagctgaagctgaagagacagctcgaatacgaggaacaggctttccaggacctcagtggtggcgaccctcctggtggaagcacatctcacctgatgtggaagcggatgaagaatctgagaggcggctcctgtcctctgatgcccgataagcctctgagcgccaacgtgcccaacgacaagttcacacagaaccccatgagaggactgggacaccctctgaggcatctgccattgccacagcctccaagcgccatttctcccggcgagaacagcaagagcagattccctccacagtgctacgccacacagtatcaggactacagcctgtctagcgcccacaaagtgtccggcatggcctcaagactgctcggccctagcttcgagagctacctgctgcctgagctgaccacatacgactgcgaagtgaacgtgcccgtgctgggctctagcacacttcttcaaggcggcgacctgctgagagcactggaccaggcaacatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgagggtacatgcgccttactccttgtgagcgcatcgcgataagctctagag

SEQ ID NO: 30 - DNA, 3360 bp, synthetic construct
  source 1..3360: mol_type = other DNA
gttacaatatcccgtgaagcttgagtggaatcaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatggactacaaggaccacgacggcgattacaaagaccatgacatcgactataaggacgatgacgacaaaggcagcggcatggaaggcgctggcggagccaacgacaagaagaagattagcagcgagcggcggaaagagaagtccagggacgctgccagaagcaggcggagcaaagaaagcgaggtgttctacgagctggcccaccaactgcctctgcctcacaatgtgtctagccacctggacaaggccagcgtgatgagactgaccatcagctacctgagagtgcggaagctgctggatgctggcgacctggacatcgaggacgatatgaaggcccagatgaactgcttctacctgaaggccctggacggcttcgtgatggtgctgaccgatgacggcgacatgatatacattagcgacaacgtgaacaagtatatggggctgacccagttcgagctgaccggccactccgtgttcgacttcacacacccttgcgaccacgaagagatgagagagatgctgacccaccggaacggcctggtcaagaagggcaaagagcagaatacccagcggtcattcttcctgcggatgaaatgcaccctgaccagccggggcagaaccatgaatatcaagagcgccacttggaaagtgctgcactgcaccggccacatccatgtgtatgacaccaacagcaaccagcctcagtgcggctacaagaaacctcctatgacctgcctggtgctgatttgcgagcccattcctcatcctagcaatatcgagattcccctggacagcaagaccttcctgagcagacacagcctggacatgaagtttagctactgcgacgagagaatcaccgagctgatgggctacgagcccgaggaacttctcggacggtccatctacgagtattatcacgccctggactccgaccacctgaccaagacaccacacgatatgttcaccaagggccaagtgaccaccggccagtatagaatgctggctaagcgcggaggctatgtgtgggttgagacacaggccaccgtcatctacaacaccaagaactcccagccacagtgcatcgtgtgcgtgaactacgtcgtgtccggcatcatccagcacgacctgattttcagcctgcaacagaccgagtgcgtgctgaagcctgtggaaagcagcgacatgaagatgacccagctttttaccaaggtggaatccgaggacaccagcagcctgttcgacaagctgaagaaagagcccgacgctctgacactgctggctccagcagcaggcgataccatcatcagcctggatttcggcagcaacgacaccgagacagacgaccagcagctcgaagaggtgcccctgtataacgatgtgatgctgccctctccaaacgagaagctccagaacatcaacctggctatgagccctctgcctaccgccgagacacctaagcctctgagaagctctgctgaccccgctctgaatcaagaagtggccctgaagctggaacccaatcctgagagcctggaactgagcttcaccatgcctcagattcaggaccagacaccttctccaagcgacggcagcacaagacagtctagccccgagcctaacagccccagcgagtattgcttctacgtggacagcgatatggtcaatgagttcaaactggaactggtcgagaagctgttcgccgaagataccgaggccaagaatcccttcagcacccaagacactgacctggacctggaaatgctggccccttacatccctatggacgacgactttcagctccggtccttcgaccagctcagccctctggaatccagctctgcctctccagaaagcgcctctccacagagcaccgtgaccgtgtttcagcagacccagattcaagagcccaccgccaatgccaccaccacaaccgccacaacagacgagctgaaaaccgtgaccaaggaccgcatggaggacatcaagattctgattgcctctccttctccgacgcacatccacaaagagacaaccagcgccacaagcagcccctacagagacacccagagcagaaccgcctctcctaatagagccggaaagggcgtcatcgagcagaccgagaagtctcaccctagaagccccaacgtgctgagcgtggcactgagccagagaaccacagtgcccgaagaggaactgaaccctaagattctggccctccagaacgcccagcggaagcggaaaatggaacacgacggaagcctgttccaggccgtcggaatcggaacactgctgcaacagcctgatgaccacgccgccaccacaagcctgtcttggaagagagtgaagggctgcaagtccagcgagcagaacggaatggaacagaaaaccatcatcctgattcctagcgacctggcctgtagactgctgggccagagcatggatgagagcggactgccacagctcaccagctacgactgcgaagtgaacgcccctatccagggcagcagaaacctgctccaaggcgaggaactgctgcgagcactggaccaagtgaactgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgacactgtttaacaagcccgtcagtagagcgcatcgcgataagctctagagggacagcccccccccaaagcccc

SEQ ID NO: 31 - DNA, 3500 bp, synthetic construct
  source 1..3500: mol_type = other DNA
gttacaatatcccgtgaagcttgagtggaatcaagatctggggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtctagccgccctcgctctagcagggagctctgcactccgcccgaaaagtctagcgtcgtgcaggacgtgacatctagtgtcgtgcaggacgtgacatctagtgtcgtgcaggacgtgacatctaggatccctctagagggtatataatgggggccactagtctactaccagagctctctggctaactagagaacccactgcttactggcttatcgaaattaatacgactcactatagggagtccccaagctggctagcgccaccatggactacaaggaccacgacggcgattacaaagaccatgacatcgactataaggacgatgacgacaaaggcagcggcatgaccgccgacaaagagaagaagagaagctccagcgagcggcggaaagagaagtctagggacgccgccagatgcaggcggagcaaagaaaccgaggtgttctacgagctggcccacgaactgcctctgcctcactctgtgtctagccacctggacaaggccagcatcatgcggctggctatcagcttcctgcggacccacaaactgctgagcagcgtgtgcagcgagaatgagtctgaagccgaagccgaccagcagatggacaacctgtatctgaaggccctggaaggcttcattgccgtcgtgacccaggacggcgatatgattttcctgagcgagaacatcagcaagttcatgggcctgacacaggtggaactgaccggccacagcatcttcgacttcacacacccctgcgaccacgaggaaatcagagagaacctgagcctgaagaacggcagcggctttggcaagaaaagcaaggatatgagcaccgagcgggatttcttcatgcggatgaaatgcaccgtgaccaaccggggcagaaccgtgaatctgaaatccgccacatggaaggtgctgcactgcaccggccaagtgaaagtgtataacaactgccctccacacaacagcctgtgcggctacaaagagcctctgctgagctgcctgattatcatgtgcgagcccattcagcacccctctcacatggacatccctctggacagcaagaccttcctgagcaggcacagcatggacatgaagttcacctactgcgacgaccgcatcaccgagctgattggctatcaccccgaagaactgctgggcagaagcgcctacgagttctatcacgccctggactccgagaacatgaccaagagccaccagaacctctgcaccaaaggccaggtggtgtccggccagtatcggatgcttgccaagcacggcggctatgtgtggctggaaacacagggcaccgtcatctacaaccccagaaacctccagcctcagtgcattatgtgcgtgaactacgtgctgagcgaaatcgagaagaacgatgtggtgtttagcatggaccagaccgagagcctgttcaagccccacctgatggctatgaactccatcttcgatagcagcggcaagggcgctgtgtccgagaagtccaacttcctgttcaccaagctgaaagaggaacccgaggaactggcccagcttgctcctacacctggcgacgccatcatcagcctggacttcggcaaccagaacttcgaggaaagctccgcctacggcaaggccatcctgcctccatctcaaccttgggccacagagctgagaagccacagcacacagagcgaagctggaagcctgcctgcctttacagtgccacaagctgctgcccctggcagcacaacaccaagcgcaacaagcagcagcagctcttgcagcaccccaaacagccccgaggactactacaccagcctggataacgacctgaagattgaagtcatcgagaagctgttcgctatggacaccgaggccaaggaccagtgtagcacccagaccgacttcaacgagctggacctggaaactctggccccttacatcccaatggacggcgaggatttccagctcagccctatctgccccgaagagagactgctggccgagaatcctcagagcaccccacagcactgcttcagcgccatgaccaacatcttccagcctctcgctccagtggctccacacagcccatttctgctggacaagttccagcagcaactcgaaagcaagaaaaccgagcctgagcaccggcctatgagcagcatctttttcgacgccggaagcaaagccagcctgccaccttgttgtggacaggcttctacccctctgtccagcatgggcggcaggtctaatactcagtggcctcctgaccctccactgcactttggccctacaaaatgggcagtgggcgaccagagaaccgagtttcttggagctgctccactgggaccaccagtgtctccacctcatgtgtccaccttcaagacaagaagcgccaaaggcttcggcgctagaggccctgatgtgctgtctcctgctatggtggccctgagcaacaagctgaagctgaagagacagctcgaatacgaggaacaggctttccaggacctcagtggtggcgaccctcctggtggaagcacatctcacctgatgtggaagcggatgaagaatctgagaggcggctcctgtcctctgatgcccgataagcctctgagcgccaacgtgcccaacgacaagttcacacagaaccccatgagaggactgggacaccctctgaggcatctgccattgccacagcctccaagcgccatttctcccggcgagaacagcaagagcagattccctccacagtgctacgccacacagtatcaggactacagcctgtctagcgcccacaaagtgtccggcatggcctcaagactgctcggccctagcttcgagagctacctgctgcctgagctgaccagatacgactgcgaagtgaacgtgcccgtgctgggctctagcacacttcttcaaggcggcgacctgctgagagcactggaccaggcaacatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtcaatagcaggcatgctggggatgcggtgggctctatggcttctgaggcaggtcgacactgtttaacaagcccgtcagtagagcgcatcgcgataagctctagagggacagcccccccccaaagcccccagggatg

SEQ ID NO: 32 - DNA, 266 bp, synthetic construct
  source 1..266: mol_type = other DNA
catggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctag

SEQ ID NO: 33 - DNA, 582 bp, synthetic construct
  source 1..582: mol_type = other DNA
tcagcttcctctcaggtgtgtcttgaggtgtctgttcaggttgtcgcgtctgctgaagtttctcatgcagattctacactggaatggcttctctccagtgtgtgtgcgcagatgtccggccaggtgctccttctggctaaagttcctcatacagatcctacactgaaatggcttctctcctgtatgtgttctggtgtgtctgtccaggcggtcctgtctgctgaagtttctcatacagattctgcactggaaaggtctttctccaggtctggcggtaccagagccacctccaccactacctcctcctccagacccgccaccaccggatccgagcatatccagatcaaagtcatccagtgcatcgctacccagcatgtcgaggtcgaaatcgtcgagagcgtcggagcccagcatatcgaggtcaaaatcgtccaaggcatcggaaccaagcatgtccagatcaaagtcatccagagcgtctccggagactttgcgctttttcttaggagccatcttatcgtcatcgtctttgtaatcgatgtcatgatccttgtaatcgccgtcgtggtctttgtagtccat

SEQ ID NO: 34 - DNA, 768 bp, synthetic construct
  source 1..768: mol_type = other DNA
tcacagctggaagtcatcatccattgggatgtatggagccagcatctccaggtccagacctcctccgcttcctctcaggtgtgtcttgaggtgtctgttcaggttgtcgcgtctgctgaagtttctcatgcagattctacactggaatggcttctctccagtgtgtgtgcgcagatgtccggccaggtgctccttctggctaaagttcctcatacagatcctacactgaaatggcttctctcctgtatgtgttctggtgtgtctgtccaggcggtcctgtctgctgaagtttctcatacagattctgcactggaaaggtctttctccaggtctggcggtaccagagccacctccaccactacctcctcctcccagctggaagtcatcatccattgggatgtatggagccagcatctccaggtccagactcccgccaccaccggatccgagcatatccagatcaaagtcatccagtgcatcgctacccagcatgtcgaggtcgaaatcgtcgagagcgtcggagcccagcatatcgaggtcaaaatcgtccaaggcatcggaaccaagcatgtccagatcaaagtcatccagagcgtctccacctcccagctggaagtcatcatccattgggatgtatggagccagcatctccaggtccagacctcctccggagactttgcgctttttcttaggagccatcttatcgtcatcgtctttgtaatcgatgtcatgatccttgtaatcgccgtcgtggtctttgtagtccat

SEQ ID NO: 35 - DNA, 255 bp, synthetic construct
  source 1..255: mol_type = other DNA
catggtggcgctagcgatgagctctggtagtagactagtggcccccattatataccctctagagagcgtgtcctcactcttttagtcctcactcttgatgtcctcactctcagcgtcctcactctgactgtcctcactctttccgtcctcactcttcctgtcctcactcttcgcgtcctcactctgagtgtcctcactcttcaggtcctcactctccttgtcctcactctcggcgtcctcactctacgcgtatat

SEQ ID NO: 36 - DNA, 475 bp, synthetic construct
  source 1..475: mol_type = other DNA
catggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagatgtacactacctggtcctcactcttttagtcctcactcttgatgtcctcactctcagcgtcctcactctgactgtcctcactctttccgtcctcactcttcctgtcctcactcttcgcgtcctcactctgagtgtcctcactcttcaggtcctcactctccttgtcctcactctcggcgtcctcactctacgcgtatatctggcccgt

SEQ ID NO: 37 - DNA, 2380 bp, synthetic construct
  source 1..2380: mol_type = other DNA
gcggggggagcgccggaccggagcggagccccgggcggctcgctgctgccccctagcgggggagggacgtaattacatccctgggggctttgggggggggctgtccctctagagcttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcattggaacacggtcactgtgctctgtggagaggcggattcaggagaggcgctagagctttccagagggctgagctggtcgaaggaccggagctggaagtcgtcgtccatagggatgtaaggggccagcatttccaggtccaggtcggtgtcttgggtgctgaagggattcttggcctcggtgtcctcggcgaacagcttctcgaccagttccagtttgaactcattgaccatgtcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctgagaggcttaggtgtctcggcggtaggcagagggctcatagccaggttgatgttctggagcttctcatttggagagggcagcatcacatcattatacaggggcacctcttcgagctgctggtcgtctgtctcggtgtcgttgctgccgaagtccaggctgatgatggtatcgcctgctgctggtgctcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtccctgcctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgccatcatgcagacagtagccgtcgtggctcagagggcaatcgttgaagtgggacaccactccgccttgtccatgccaggctgtagcaaccagcagaagcagcagaacgccagggcccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 38 - DNA, 2940 bp, synthetic construct
  source 1..2940: mol_type = other DNA
gcgccggaccggagcggagccccgggcggctcgctgctgccccctagcgggggagggacgtaattacatccctgggggctttgggggggggctgtccctctagagcttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcatcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtccctgcctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgccatcatgcagacagtagccgtcgtggctcagagggcaatcgttgaagtgggacaccactccgccttgtccatgccaggctgtagcaaccagcagaagcagcagaacgccagggcccatggtggcgctagcctcgagaatttggatcggtcccgttctcttctatggaggtcaaaacagcgtggatggcgtctccaggcgatctgacggttcactaaacgagctctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaagccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactcacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 39 - DNA, 1680 bp, synthetic construct
  source 1..1680: mol_type = other DNA
gggggggggctgtccctctagagcttatcgcgatgcgctcacaaggagtaaggcgcatgtaccctcgacctgcctcagaagccatagagcccaccgcatccccagcatgcctgctattgacttcccaatcctcccccttgctgtcctgccccccccatccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcatcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtccctgcctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgccatcatgcagacagtagccgtcgtggctcagagggcaatcgttgaagtgggacaccactccgccttgtccatgccaggctgtagcaaccagcagaagcagcagaacgccagggcccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 40 - DNA, 3500 bp, synthetic construct
  source 1..3500: mol_type = other DNA
ggggagggacgtaattacatccctgggggctttgggggggggctgtccctctagagcttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcattggaacacggtcactgtgctctgtggagaggcggattcaggagaggcgctagagctttccagagggctgagctggtcgaaggaccggagctggaagtcgtcgtccatagggatgtaaggggccagcatttccaggtccaggtcggtgtcttgggtgctgaagggattcttggcctcggtgtcctcggcgaacagcttctcgaccagttccagtttgaactcattgaccatgtcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctgagaggcttaggtgtctcggcggtaggcagagggctcatagccaggttgatgttctggagcttctcatttggagagggcagcatcacatcattatacaggggcacctcttcgagctgctggtcgtctgtctcggtgtcgttgctgccgaagtccaggctgatgatggtatcgcctgctgctggtgctcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtccctgcctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgccatcatgcagacagtagccgtcgtggctcagagggcaatcgttgaagtgggacaccactccgccttgtccatgccaggctgtagcaaccagcagaagcagcagaacgccagggcccatggtggcgctagcctcgagaatttggatcggtcccgttctcttctatggaggtcaaaacagcgtggatggcgtctccaggcgatctgacggttcactaaacgagctctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaagccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactcacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 41 - DNA, 1260 bp, synthetic construct; identical to SEQ ID NO: 23
  source 1..1260: mol_type = other DNA

SEQ ID NO: 42 - DNA, 700 bp, synthetic construct
  source 1..700: mol_type = other DNA
agccccgggcggctcgctgctgccccctagcgggggagggacgtaattacatccctgggggctttgggggggggctgtccctctagagcttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgccactgtgctggatatctgcagaattccaccacactggactagtggatccgagctcggtaccaagcttaagtttaaacgctagcctcgagaatttggatcggtcccgttctcttctatggaggtcaaaacagcgtggatggcgtctccaggcgatctgacggttcactaaacgagctctcacgacacctgaaatggaagaaaaaaactttgaaccactgtctgaggcttgagaatgaaccaagatccaaactcaaaaagggcaaattccaaggagaatta

SEQ ID NO: 43 - DNA, 4900 bp, synthetic construct
  source 1..4900: mol_type = other DNA
cttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcattggaacacggtcactgtgctctgtggagaggcggattcaggagaggcgctagagctttccagagggctgagctggtcgaaggaccggagctggaagtcgtcgtccatagggatgtaaggggccagcatttccaggtccaggtcggtgtcttgggtgctgaagggattcttggcctcggtgtcctcggcgaacagcttctcgaccagttccagtttgaactcattgaccatgtcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctgagaggcttaggtgtctcggcggtaggcagagggctcatagccaggttgatgttctggagcttctcatttggagagggcagcatcacatcattatacaggggcacctcttcgagctgctggtcgtctgtctcggtgtcgttgctgccgaagtccaggctgatgatggtatcgcctgctgctggtgctcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtccctgcctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgccatcatgcagacagtagccgtcgtggctcagagggcaatcgttgaagtgggacaccactccgccttgtccatgccaggctgtagcaaccagcagaagcagcagaacgccagggcccataggtccggggttctcttccacatctccgcatgtaagaaggctgcctctgccttctccgctgccgcttctttttctccggttcacttggtccagtgctcgcagcagttcctcgccttggagcaggtttctgctgccctggataggggcgttcacttcgcagtcgtagctggtgagctgtggcagtccgctctcatccatgctctggcccagcagtctacaggccaggtcgctaggaatcaggatgatggttttctgttccattccgttctgctcgctggacttgcagcccttcactctcttccaagacaggcttgtggtggcggcgtggtcatcaggctgttgcagcagtgttccgattccgacggcctggaacaggcttccgtcgtgttccattttccgcttccgctgggcgttctggagggccagaatcttagggttcagttcctcttcgggcactgtggttctctggctcagtgccacgctcagcacgttggggcttctagggtgagacttctcggtctgctcgatgacgccctttccggctctattaggagaggcggttctgctctgggtgtctctgtaggggctgcttgtggcgctggttgtctctttgtggatgtgcgtcggagaaggagaggcaatcagaatcttgatgtcctccatgcggtccttggtcacggttttcagctcgtctgttgtggcggttgtggtggtggcattggcggtgggctcttgaatctgggtctgctgaaacacggtcacggtgctctgtggagaggcgctttctggagaggcagagctggattccagagggctgagctggtcgaaggaccggagctgaaagtcgtcgtccatagggatgtaaggggccagcatttccaggtccaggtcagtgtcttgggtgctgaagggattcttggcctcggtatcttcggcgaacagcttctcgaccagttccagtttgaactcattgaccatatcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctcagaggcttaggtgtctcggcggtaggcagagggctcatagccaggttgatgttctggagcttctcgtttggagagggcagcatcacatcgttatacaggggcacctcttcgagctgctggtcgtctgtctcggtgtcgttgctgccgaaatccaggctgatgatggtatcgcctgctgctggagccagcagtgtcagagcgtcgggctctttcttcagcttgtcgaacaggctgctggtgtcctcggattccaccttggtaaaaagctgggtcatcttcatgtcgctgctttccacaggcttcagcacgcactcggtctgttgcaggctgaaaatcaggtcgtgctggatgatgccggacacgacgtagttcacgcacacgatgcactgtggctgggagttcttggtgttgtagatgacggtggcctgtgtctcaacccacacatagcctccgcgcttagccagcattctatactggccggtggtcacttggcccttggtgaacatatcgtggtgtgtcttggtcaggtggtcggagtccagggcgtgataatactcgtagatggaccgtccgagaagttcctcgggctcgtagcccatcagctcggtgattctctcgtcgcagtagctaaacttcatgtccaggctgtgtctgctcaggaaggtcttgctgtccaggggaatctcgatattgctaggatgaggaatgggctcgcaaatcagcaccaggcaggtcataggaggtttcttgtagccgcactgaggctggttgctgttggtgtcatacacatggatgtggccggtgcagtgcagcactttccaagtggcgctcttgatattcatggttctgccccggctggtcagggtgcatttcatccgcaggaagaatgaccgctgggtattctgctctttgcccttcttgaccaggccgttccggtgggtcagcatctctctcatctcttcgtggtcgcaagggtgtgtgaagtcgaacacggagtggccggtcagctcgaactgggtcagccccatatacttgttcacgttgtcgctaatgtatatcatgtcgccgtcatcggtcagcaccatcacgaagccgtccagggccttcaggtagaagcagttcatctgggccttcatatcgtcctcgatgtccaggtcgccagcatccagcagcttccgcactctcaggtagctgatggtcagtctcatcacgctggccttgtccaggtggctagacacattgtgaggcagaggcagttggtgggccagctcgtagaacacctcgctttctttgctccgcctgcttctggcagcgtccctggacttctctttccgccgctcgctgctaatcttcttcttgtcgttggctccgccagcgccttccatgccgctgcctttgtcgtcatcgtccttatagtcgatgtcatggtctttgtaatcgccgtcgtggtccttgtagtccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 44 - DNA, 3780 bp, synthetic construct
  source 1..3780: mol_type = other DNA
taaggggccagcatttccaggtccaggtcagtgtcttgggtgctgaagggattcttggcctcggtatcttcggcgaacagcttctcgaccagttccagtttgaactcattgaccatatcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctcagaggcttaggtgtctcggcggtaggcagagggctcatagccaggttgatgttctggagcttctcgtttggagagggcagcatcacatcgttatacaggggcacctcttcgagctgctggtcgtctgtctcggtgtcgttgctgccgaaatccaggctgatgatggtatcgcctgctgctggagccagcagtgtcagagcgtcgggctctttcttcagcttgtcgaacaggctgctggtgtcctcggattccaccttggtaaaaagctgggtcatcttcatgtcgctgctttccacaggcttcagcacgcactcggtctgttgcaggctgaaaatcaggtcgtgctggatgatgccggacacgacgtagttcacgcacacgatgcactgtggctgggagttcttggtgttgtagatgacggtggcctgtgtctcaacccacacatagcctccgcgcttagccagcattctatactggccggtggtcacttggcccttggtgaacatatcgtggtgtgtcttggtcaggtggtcggagtccagggcgtgataatactcgtagatggaccgtccgagaagttcctcgggctcgtagcccatcagctcggtgattctctcgtcgcagtagctaaacttcatgtccaggctgtgtctgctcaggaaggtcttgctgtccaggggaatctcgatattgctaggatgaggaatgggctcgcaaatcagcaccaggcaggtcataggaggtttcttgtagccgcactgaggctggttgctgttggtgtcatacacatggatgtggccggtgcagtgcagcactttccaagtggcgctcttgatattcatggttctgccccggctggtcagggtgcatttcatccgcaggaagaatgaccgctgggtattctgctctttgcccttcttgaccaggccgttccggtgggtcagcatctctctcatctcttcgtggtcgcaagggtgtgtgaagtcgaacacggagtggccggtcagctcgaactgggtcagccccatatacttgttcacgttgtcgctaatgtatatcatgtcgccgtcatcggtcagcaccatcacgaagccgtccagggccttcaggtagaagcagttcatctgggccttcatatcgtcctcgatgtccaggtcgccagcatccagcagcttccgcactctcaggtagctgatggtcagtctcatcacgctggccttgtccaggtggctagacacattgtgaggcagaggcagttggtgggccagctcgtagaacacctcgctttctttgctccgcctgcttctggcagcgtccctggacttctctttccgccgctcgctgctaatcttcttcttgtcgttggctccgccagcgccttccatgccgctgcctttgtcgtcatcgtccttatagtcgatgtcatggtctttgtaatcgccgtcgtggtccttgtagtccataggtccggggttctcttccacatctccgcatgtaagaaggctgcctctgccttctccgctgccgcttctttttctccgttggaacacggtcactgtgctctgtggagaggcggattcaggagaggcgctagagctttccagagggctgagctggtcgaaggaccggagctggaagtcgtcgtccatagggatgtaaggggccagcatttccaggtccaggtcggtgtcttgggtgctgaagggattcttggcctcggtgtcctcggcgaacagcttctcgaccagttccagtttgaactcattgaccatgtcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctgagaggcttaggtgtctcggcggtaggcagagggctcatagccaggttgatgttctggagcttctcatttggagagggcagcatcacatcattatacaggggcacctcttcgagctgctggtcgtctgtctcggtgtcgttgctgccgaagtccaggctgatgatggtatcgcctgctgctggtgctcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtccctgcctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgccatcatgcagacagtagccgtcgtggctcagagggcaatcgttgaagtgggacaccactccgccttgtccatgccaggctgtagcaaccagcagaagcagcagaacgccagggcccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 45 - DNA, 980 bp, synthetic construct
  source 1..980: mol_type = other DNA
cttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcagttcacttggtccagtgctcgcagcagttcctcgccttggagcaggtttctgctgccctggataggggcgttcacttcgcagtcgtagctggtgagctgtggcagtccgctctcatccatgctctggcccagcagtctacaggccaggtcgctaggaatcaggatgatggttttctgttccattccgttctgctcgctggacttgcagcccttcactctcttccaagacaggcttgtggtggcggcgtggtcatcaggctgttgcagcagtgttccgattccgacggcctggaacaggcttccgtcgtgttccattttccgcttccgctgggcgttctggagggccagaatcttagggttcagttcctcttcgggcactgtggttctctggctcagtgccacgctcagcacgttggggcttctagggtgagacttctcggtctgctcgatgacgccctttccggctctattaggagaggcggttctgctctgggtgtctctgtaggggctgcttgtggcgctggttgtctctttgtggatgtgcgtcggagaaggagaggcaatcagaatcttgatgtcctccatgcggtccttggtcacggttttcagctcgtctgttgtggcggttgtggtggtggcattgg

SEQ ID NO: 46 - DNA, 5040 bp, synthetic construct
  source 1..5040: mol_type = other DNA
ctctagagcttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcattggaacacggtcactgtgctctgtggagaggcggattcaggagaggcgctagagctttccagagggctgagctggtcgaaggaccggagctggaagtcgtcgtccatagggatgtaaggggccagcatttccaggtccaggtcggtgtcttgggtgctgaagggattcttggcctcggtgtcctcggcgaacagcttctcgaccagttccagtttgaactcattgaccatgtcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctgagaggcttaggtgtctcggcggtaggcagagggctcatagccaggttgatgttctggagcttctcatttggagagggcagcatcacatcattatacaggggcacctcttcgagctgctggtcgtctgtctcggtgtcgttgctgccgaagtccaggctgatgatggtatcgcctgctgctggtgctcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtccctgcctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgccatcatgcagacagtagccgtcgtggctcagagggcaatcgttgaagtgggacaccactccgccttgtccatgccaggctgtagcaaccagcagaagcagcagaacgccagggcccataggtccggggttctcttccacatctccgcatgtaagaaggctgcctctgccttctccgctgccgcttctttttctccgtgttgcctggtccagtgctctcagcaggtcgccgccttgaagaagtgtgctagagcccagcacgggcacgttcacttcgcagtcgtatctggtcagctcaggcagcaggtagctctcgaagctagggccgagcagtcttgaggccatgccggacactttgtgggcgctagacaggctgtagtcctgatactgtgtggcgtagcactgtggagggaatctgctcttgctgttctcgccgggagaaatggcgcttggaggctgtggcaatggcagatgcctcagagggtgtcccagtcctctcatggggttctgtgtgaacttgtcgttgggcacgttggcgctcagaggcttatcgggcatcagaggacaggagccgcctctcagattcttcatccgcttccacatcaggtgagatgtgcttccaccaggagggtcgccaccactgaggtcctggaaagcctgttcctcgtattcgagctgtctcttcagcttcagcttgttgctcagggccaccatagcaggagacagcacatcagggcctctagcgccgaagcctttggcgcttcttgtcttgaaggtggacacatgaggtggagacactggtggtcccagtggagcagctccaagaaactcggttctctggtcgcccactgcccattttgtagggccaaagtgcagtggagggtcaggaggccactgagtattagacctgccgcccatgctggacagaggggtagaagcctgtccacaacaaggtggcaggctggctttgcttccggcgtcgaaaaagatgctgctcataggccggtgctcaggctcggttttcttgctttcgagttgctgctggaacttgtccagcagaaatgggctgtgtggagccactggagcgagaggctggaagatgttggtcatggcgctgaagcagtgctgtggggtgctctgaggattctcggccagcagtctctcttcggggcagatagggctgagctggaaatcctcgccgtccattgggatgtaaggggccagagtttccaggtccagctcgttgaagtcggtctgggtgctacactggtccttggcctcggtgtccatagcgaacagcttctcgatgacttcaatcttcaggtcgttatccaggctggtgtagtagtcctcggggctgtttggggtgctgcaagagctgctgctgcttgttgcgcttggtgttgtgctgccaggggcagcagcttgtggcactgtaaaggcaggcaggcttccagcttcgctctgtgtgctgtggcttctcagctctgtggcccaaggttgagatggaggcaggatggccttgccgtaggcggagctttcctcgaagttctggttgccgaagtccaggctgatgatggcgtcgccaggtgtaggagcaagctgggccagttcctcgggttcctctttcagcttggtgaacaggaagttggacttctcggacacagcgcccttgccgctgctatcgaagatggagttcatagccatcaggtggggcttgaacaggctctcggtctggtccatgctaaacaccacatcgttcttctcgatttcgctcagcacgtagttcacgcacataatgcactgaggctggaggtttctggggttgtagatgacggtgccctgtgtttccagccacacatagccgccgtgcttggcaagcatccgatactggccggacaccacctggcctttggtgcagaggttctggtggctcttggtcatgttctcggagtccagggcgtgatagaactcgtaggcgcttctgcccagcagttcttcggggtgatagccaatcagctcggtgatgcggtcgtcgcagtaggtgaacttcatgtccatgctgtgcctgctcaggaaggtcttgctgtccagagggatgtccatgtgagaggggtgctgaatgggctcgcacatgataatcaggcagctcagcagaggctctttgtagccgcacaggctgttgtgtggagggcagttgttatacactttcacttggccggtgcagtgcagcaccttccatgtggcggatttcagattcacggttctgccccggttggtcacggtgcatttcatccgcatgaagaaatcccgctcggtgctcatatccttgcttttcttgccaaagccgctgccgttcttcaggctcaggttctctctgatttcctcgtggtcgcaggggtgtgtgaagtcgaagatgctgtggccggtcagttccacctgtgtcaggcccatgaacttgctgatgttctcgctcaggaaaatcatatcgccgtcctgggtcacgacggcaatgaagccttccagggccttcagatacaggttgtccatctgctggtcggcttcggcttcagactcattctcgctgcacacgctgctcagcagtttgtgggtccgcaggaagctgatagccagccgcatgatgctggccttgtccaggtggctagacacagagtgaggcagaggcagttcgtgggccagctcgtagaacacctcggtttctttgctccgcctgcatctggcggcgtccctagacttctctttccgccgctcgctggagcttctcttcttctctttgtcggcggtcatgccgctgcctttgtcgtcatcgtccttatagtcgatgtcatggtctttgtaatcgccgtcgtggtccttgtagtccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 47 - DNA, 5040 bp, synthetic construct
  source 1..5040: mol_type = other DNA
ctctagagcttatcgcgatgcgctcacaaggagtaaggcgcatgtaccctcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcatgttgcctggtccagtgctctcagcaggtcgccgccttgaagaagtgtgctagagcccagcacgggcacgttcacttcgcagtcgtatgtggtcagctcaggcagcaggtagctctcgaagctagggccgagcagtcttgaggccatgccggacactttgtgggcgctagacaggctgtagtcctgatactgtgtggcgtagcactgtggagggaatctgctcttgctgttctcgccgggagaaatggcgcttggaggctgtggcaatggcagatgcctcagagggtgtcccagtcctctcatggggttctgtgtgaacttgtcgttgggcacgttggcgctcagaggcttatcgggcatcagaggacaggagccgcctctcagattcttcatccgcttccacatcaggtgagatgtgcttccaccaggagggtcgccaccactgaggtcctggaaagcctgttcctcgtattcgagctgtctcttcagcttcagcttgttgctcagggccaccatagcaggagacagcacatcagggcctctagcgccgaagcctttggcgcttcttgtcttgaaggtggacacatgaggtggagacactggtggtcccagtggagcagctccaagaaactcggttctctggtcgcccactgcccattttgtagggccaaagtgcagtggagggtcaggaggccactgagtattagacctgccgcccatgctggacagaggggtagaagcctgtccacaacaaggtggcaggctggctttgcttccggcgtcgaaaaagatgctgctcataggccggtgctcaggctcggttttcttgctttcgagttgctgctggaacttgtccagcagaaatgggctgtgtggagccactggagcgagaggctggaagatgttggtcatggcgctgaagcagtgctgtggggtgctctgaggattctcggccagcagtctctcttcggggcagatagggctgagctggaaatcctcgccgtccattgggatgtaaggggccagagtttccaggtccagctcgttgaagtcggtctgggtgctacactggtccttggcctcggtgtccatagcgaacagcttctcgatgacttcaatcttcaggtcgttatccaggctggtgtagtagtcctcggggctgtttggggtgctgcaagagctgctgctgcttgttgcgcttggtgttgtgctgccaggggcagcagcttgtggcactgtaaaggcaggcaggcttccagcttcgctctgtgtgctgtggcttctcagctctgtggcccaaggttgagatggaggcaggatggccttgccgtaggcggagctttcctcgaagttctggttgccgaagtccaggctgatgatggcgtcgccaggtgtaggagcaagctgggccagttcctcgggttcctctttcagcttggtgaacaggaagttggacttctcggacacagcgcccttgccgctgctatcgaagatggagttcatagccatcaggtggggcttgaacaggctctcggtctggtccatgctaaacaccacatcgttcttctcgatttcgctcagcacgtagttcacgcacataatgcactgaggctggaggtttctggggttgtagatgacggtgccctgtgtttccagccacacatagccgccgtgcttggcaagcatccgatactggccggacaccacctggcctttggtgcagaggttctggtggctcttggtcatgttctcggagtccagggcgtgatagaactcgtaggcgcttctgcccagcagttcttcggggtgatagccaatcagctcggtgatgcggtcgtcgcagtaggtgaacttcatgtccatgctgtgcctgctcaggaaggtcttgctgtccagagggatgtccatgtgagaggggtgctgaatgggctcgcacatgataatcaggcagctcagcagaggctctttgtagccgcacaggctgttgtgtggagggcagttgttatacactttcacttggccggtgcagtgcagcaccttccatgtggcggatttcagattcacggttctgccccggttggtcacggtgcatttcatccgcatgaagaaatcccgctcggtgctcatatccttgcttttcttgccaaagccgctgccgttcttcaggctcaggttctctctgatttcctcgtggtcgcaggggtgtgtgaagtcgaagatgctgtggccggtcagttccacctgtgtcaggcccatgaacttgctgatgttctcgctcaggaaaatcatatcgccgtcctgggtcacgacggcaatgaagccttccagggccttcagatacaggttgtccatctgctggtcggcttcggcttcagactcattctcgctgcacacgctgctcagcagtttgtgggtccgcaggaagctgatagccagccgcatgatgctggccttgtccaggtggctagacacagagtgaggcagaggcagttcgtgggccagctcgtagaacacctcggtttctttgctccgcctgcatctggcggcgtccctagacttctctttccgccgctcgctggagcttctcttcttctctttgtcggcggtcatgccgctgcctttgtcgtcatcgtccttatagtcgatgtcatggtctttgtaatcgccgtcgtggtccttgtagtccataggtccggggttctcttccacatctccgcatgtaagaaggctgcctctgccttctccgctgccgcttctttttctccgttggaacacggtcactgtgctctgtggagaggcggattcaggagaggcgctagagctttccagagggctgagctggtcgaaggaccggagctggaagtcgtcgtccatagggatgtaaggggccagcatttccaggtccaggtcggtgtcttgggtgctgaagggattcttggcctcggtgtcctcggcgaacagcttctcgaccagttccagtttgaactcattgaccatgtcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctgagaggcttaggtgtctcggcggtaggcagagggctcatagccaggttgatgttctggagcttctcatttggagagggcagcatcacatcattatacaggggcacctcttcgagctgctggtcgtctgtctcggtgtcgttgctgccgaagtccaggctgatgatggtatcgcctgctgctggtgctcttggaggcagggcttgcatgtgcagggcatcataggtgtccttggtggcggtgctgagtccctgatacaggccgtcgtgtcccttgcctcttctgcgttcgcccttcatgccaatctcgctgtaggcttcggccatcttgtccttttgcagctcgttatacaggccctcttgaggattcttccgtctgggctttccgcccatctcagggtcctgccctctccgcttatccagcacatcatactcttctctgcgccccaggttcagctcgttgtagagctggttctgtccctgctgataggcaggagcgtcggcgcttctggagaacttcactctgctccgataggcggcgaagtctctaggtggagcgtaaggctggtagtgctttctggttggtccgggccgtctaggggtcatgttcatgtagtcgctgtgcagcagtctgctccgcttgcttcggacccaaaagatgatgaaggccactgtaaccagcaggctgtaacaggccagcacgccgccaacaacaacgagcacccagaaaggcttgctaggtccggggaacagtggagaaggacacaggtgcttgcccttcacgtggatgatggtgccgttggacttctcgttgtccaggtaaggtggagggtacatcacttcgatggcagcggctctcagctcccaccacttcaggtcccgatactggcatctctcgccgatgtagcccacgacgcagttgcaggcgtatttgtccagggcctcgatatacatgcacacgcatccatgcagacagtagccgtcgtggctcagagggcaatcgttgaagtgggacaccactccgccttgtccatgccaggctgtagcaaccagcagaagcagcagaacgccagggcccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgtactagcactcgtttgtcaagacc

SEQ ID NO: 48 - DNA, 3360 bp, synthetic construct
  source 1..3360: mol_type = other DNA
ggggctttgggggggggctgtccctctagagcttatcgcgatgcgctctactgacgggcttgttaaacagtgtcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcagttcacttggtccagtgctcgcagcagttcctcgccttggagcaggtttctgctgccctggataggggcgttcacttcgcagtcgtagctggtgagctgtggcagtccgctctcatccatgctctggcccagcagtctacaggccaggtcgctaggaatcaggatgatggttttctgttccattccgttctgctcgctggacttgcagcccttcactctcttccaagacaggcttgtggtggcggcgtggtcatcaggctgttgcagcagtgttccgattccgacggcctggaacaggcttccgtcgtgttccattttccgcttccgctgggcgttctggagggccagaatcttagggttcagttcctcttcgggcactgtggttctctggctcagtgccacgctcagcacgttggggcttctagggtgagacttctcggtctgctcgatgacgccctttccggctctattaggagaggcggttctgctctgggtgtctctgtaggggctgcttgtggcgctggttgtctctttgtggatgtgcgtcggagaaggagaggcaatcagaatcttgatgtcctccatgcggtccttggtcacggttttcagctcgtctgttgtggcggttgtggtggtggcattggcggtgggctcttgaatctgggtctgctgaaacacggtcacggtgctctgtggagaggcgctttctggagaggcagagctggattccagagggctgagctggtcgaaggaccggagctgaaagtcgtcgtccatagggatgtaaggggccagcatttccaggtccaggtcagtgtcttgggtgctgaagggattcttggcctcggtatcttcggcgaacagcttctcgaccagttccagtttgaactcattgaccatatcgctgtccacgtagaagcaatactcgctggggctgttaggctcggggctagactgtcttgtgctgccgtcgcttggagaaggtgtctggtcctgaatctgaggcatggtgaagctcagttccaggctctcaggattgggttccagcttcagggccacttcttgattcagagcggggtcagcagagcttctcagaggcttaggtgtctcggcggtaggcagagggctcatagccaggttgatgttctggagcttctcgtttggagagggcagcatcacatcgttatacaggggcacctcttcgagctgctggtcgtctgtctcggtgtcgttgctgccgaaatccaggctgatgatggtatcgcctgctgctggagccagcagtgtcagagcgtcgggctctttcttcagcttgtcgaacaggctgctggtgtcctcggattccaccttggtaaaaagctgggtcatcttcatgtcgctgctttccacaggcttcagcacgcactcggtctgttgcaggctgaaaatcaggtcgtgctggatgatgccggacacgacgtagttcacgcacacgatgcactgtggctgggagttcttggtgttgtagatgacggtggcctgtgtctcaacccacacatagcctccgcgcttagccagcattctatactggccggtggtcacttggcccttggtgaacatatcgtgtggtgtcttggtcaggtggtcggagtccagggcgtgataatactcgtagatggaccgtccgagaagttcctcgggctcgtagcccatcagctcggtgattctctcgtcgcagtagctaaacttcatgtccaggctgtgtctgctcaggaaggtcttgctgtccaggggaatctcgatattgctaggatgaggaatgggctcgcaaatcagcaccaggcaggtcataggaggtttcttgtagccgcactgaggctggttgctgttggtgtcatacacatggatgtggccggtgcagtgcagcactttccaagtggcgctcttgatattcatggttctgccccggctggtcagggtgcatttcatccgcaggaagaatgaccgctgggtattctgctctttgcccttcttgaccaggccgttccggtgggtcagcatctctctcatctcttcgtggtcgcaagggtgtgtgaagtcgaacacggagtggccggtcagctcgaactgggtcagccccatatacttgttcacgttgtcgctaatgtatatcatgtcgccgtcatcggtcagcaccatcacgaagccgtccagggccttcaggtagaagcagttcatctgggccttcatatcgtcctcgatgtccaggtcgccagcatccagcagcttccgcactctcaggtagctgatggtcagtctcatcacgctggccttgtccaggtggctagacacattgtgaggcagaggcagttggtgggccagctcgtagaacacctcgctttctttgctccgcctgcttctggcagcgtccctggacttctctttccgccgctcgctgctaatcttcttcttgtcgttggctccgccagcgccttccatgccgctgcctttgtcgtcatcgtccttatagtcgatgtcatggtctttgtaatcgccgtcgtggtccttgtagtccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgattccactcaagcttcacgggatattgtaac

SEQ ID NO: 49 - DNA, 3500 bp, synthetic construct
  source 1..3500: mol_type = other DNA
catccctgggggctttgggggggggctgtccctctagagcttatcgcgatgcgctctactgacgggcttgttaaacagtgtcgacctgcctcagaagccatagagcccaccgcatccccagcatgcctgctattgacttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagcggccgctcatgttgcctggtccagtgctctcagcaggtcgccgccttgaagaagtgtgctagagcccagcacgggcacgttcacttcgcagtcgtatctggtcagctcaggcagcaggtagctctcgaagctagggccgagcagtcttgaggccatgccggacactttgtgggcgctagacaggctgtagtcctgatactgtgtggcgtagcactgtggagggaatctgctcttgctgttctcgccgggagaaatggcgcttggaggctgtggcaatggcagatgcctcagagggtgtcccagtcctctcatggggttctgtgtgaacttgtcgttgggcacgttggcgctcagaggcttatcgggcatcagaggacaggagccgcctctcagattcttcatccgcttccacatcaggtgagatgtgcttccaccaggagggtcgccaccactgaggtcctggaaagcctgttcctcgtattcgagctgtctcttcagcttcagcttgttgctcagggccaccatagcaggagacagcacatcagggcctctagcgccgaagcctttggcgcttcttgtcttgaaggtggacacatgaggtggagacactggtggtcccagtggagcagctccaagaaactcggttctctggtcgcccactgcccattttgtagggccaaagtgcagtggagggtcaggaggccactgagtattagacctgccgcccatgctggacagaggggtagaagcctgtccacaacaaggtggcaggctggctttgcttccggcgtcgaaaaagatgctgctcataggccggtgctcaggctcggttttcttgctttcgagttgctgctggaacttgtccagcagaaatgggctgtgtggagccactggagcgagaggctggaagatgttggtcatggcgctgaagcagtgctgtggggtgctctgaggattctcggccagcagtctctcttcggggcagatagggctgagctggaaatcctcgccgtccattgggatgtaaggggccagagtttccaggtccagctcgttgaagtcggtctgggtgctacactggtccttggcctcggtgtccatagcgaacagcttctcgatgacttcaatcttcaggtcgttatccaggctggtgtagtagtcctcggggctgtttggggtgctgcaagagctgctgctgcttgttgcgcttggtgttgtgctgccaggggcagcagcttgtggcactgtaaaggcaggcaggcttccagcttcgctctgtgtgctgtggcttctcagctctgtggcccaaggttgagatggaggcaggatggccttgccgtaggcggagctttcctcgaagttctggttgccgaagtccaggctgatgatggcgtcgccaggtgtaggagcaagctgggccagttcctcgggttcctctttcagcttggtgaacaggaagttggacttctcggacacagcgcccttgccgctgctatcgaagatggagttcatagccatcaggtggggcttgaacaggctctcggtctggtccatgctaaacaccacatcgttcttctcgatttcgctcagcacgtagttcacgcacataatgcactgaggctggaggtttctggggttgtagatgacggtgccctgtgtttccagccacacatagccgccgtgcttggcaagcatccgatactggccggacaccacctggcctttggtgcagaggttctggtggctcttggtcatgttctcggagtccagggcgtgatagaactcgtaggcgcttctgcccagcagttcttcggggtgatagccaatcagctcggtgatgcggtcgtcgcagtaggtgaacttcatgtccatgctgtgcctgctcaggaaggtcttgctgtccagagggatgtccatgtgagaggggtgctgaatgggctcgcacatgataatcaggcagctcagcagaggctctttgtagccgcacaggctgttgtgtggagggcagttgttatacactttcacttggccggtgcagtgcagcaccttccatgtggcggatttcagattcacggttctgccccggttggtcacggtgcatttcatccgcatgaagaaatcccgctcggtgctcatatccttgcttttcttgccaaagccgctgccgttcttcaggctcaggttctctctgatttcctcgtggtcgcaggggtgtgtgaagtcgaagatgctgtggccggtcagttccacctgtgtcaggcccatgaacttgctgatgttctcgctcaggaaaatcatatcgccgtcctgggtcacgacggcaatgaagccttccagggccttcagatacaggttgtccatctgctggtcggcttcggcttcagactcattctcgctgcacacgctgctcagcagtttgtgggtccgcaggaagctgatagccagccgcatgatgctggccttgtccaggtggctagacacagagtgaggcagaggcagttcgtgggccagctcgtagaacacctcggtttctttgctccgcctgcatctggcggcgtccctagacttctctttccgccgctcgctggagcttctcttcttctctttgtcggcggtcatgccgctgcctttgtcgtcatcgtccttatagtcgatgtcatggtctttgtaatcgccgtcgtggtccttgtagtccatggtggcgctagccagcttggggactccctatagtgagtcgtattaatttcgataagccagtaagcagtgggttctctagttagccagagagctctggtagtagactagtggcccccattatataccctctagagggatcctagatgtcacgtcctgcacgacactagatgtcacgtcctgcacgacactagatgtcacgtcctgcacgacgctagacttttcgggcggagtgcagagctccctgctagagcgagggcggctagacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccccagatcttgattccactcaagcttcacgggatattgtaac